(19) United States
(12) Patent Application Publication
(10) Pub. No.: US 2026/0000596 A1
(43) Pub. Date: Jan. 1, 2026

(54) SEMI-SYNTHETIC RHAMNOLIPIDS

(71) Applicant: The Procter & Gamble Company, Cincinnati, OH (US)

(72) Inventors: John August WOS, Mason, OH (US); Hilda Andamiche NAMANJA-MAGLIANO, Loveland, OH (US); Jose Carlos GARCIA-GARCIA, Cincinnati, OH (US); Howard David HUTTON, III, Oregonia, OH (US); Isoken Omosefe IGWEKALA-NWEKE, Springdale, OH (US); Amanda Brooke BRODBECK, Cincinnati, OH (US); Ryan Michael WEST, West Chester, OH (US); Dakota James BROCK, West Chester, OH (US); Victor Manuel ARREDONDO, Madeira, OH (US); Lijuan LI, Lebanon, OH (US); Sydney Anne McKEE, Mason, OH (US); Gary Richard FUENTES, Batesville, IN (US); Colleen Marie NEAL, Cincinnati, OH (US)

(21) Appl. No.: 19/062,608

(22) Filed: Feb. 25, 2025

Related U.S. Application Data
(60) Provisional application No. 63/557,812, filed on Feb. 26, 2024.
(60) Provisional application No. 63/558,670, filed on Feb. 28, 2024.
(60) Provisional application No. 63/712,718, filed on Oct. 28, 2024.

(51) Int. Cl. A61K 8/60; A01N 43/16; A01P 1/00; A61K 8/365; A61Q 5/02

(52) U.S. Cl. CPC A61K 8/60; A01N 43/16; A01P 1/00; A61K 8/365; A61K 2800/33; A61Q 5/02

(57) ABSTRACT

A composition comprising rhamnolipid compositions, said rhamnolipid compositions comprising (a) and (b), and said composition further comprising (c), wherein (a), (b), and (c) are as follows:

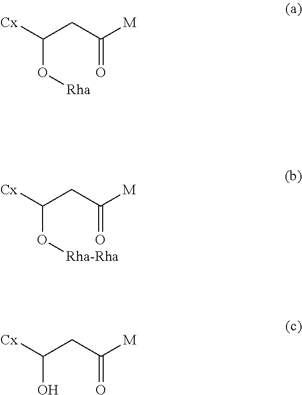

wherein Rha is rhamnose;
wherein each Cx is independently selected from an alkyl, aryl, heteroalkyl, heteroaryl, unsaturated alkenyl, and unsaturated heteroalkenyl;
wherein each Cx independently has a carbon chain length from 4 to 22;
wherein each M is independently selected from OH; O−X+ wherein X+ is a cation; OR1; alkyl, heteroalkyl; aryl; heteroaryl; hetero arylalkyl; arylalkyl; and tauryl;
wherein R1 is selected from an alkyl, branched alkyl, and cyclic alkyl;
and combinations thereof; and all possible stereoisomers thereof; and
wherein (a) or (b) is at least about 25 wt % or greater of all rhamnolipids present in the compositions.

### CROSS REFERENCE TO RELATED APPLICATIONS

This application claims the benefit, under 35 U.S.C. § 119 (e), to U.S. Provisional Application Nos. 63/712,718 filed Oct. 28, 2024, 63/558,670 filed Feb. 28, 2024, and 63/557,812, filed Feb. 26, 2024, the entire disclosure of which is fully incorporated by reference herein.

### FIELD OF THE INVENTION

The present disclosure generally relates to rhamnolipids modified such as through hydrolysis.

### BACKGROUND OF THE INVENTION

Consumers are seeking more natural and milder cleansers without performance trade-offs. Rhamnolipids, a subset of glycolipids, are known and can fulfill certain consumer needs by offering natural surfactants while still providing foaming and lathering. However, commercial rhamnolipids tend to be costly, and many are still low performing compared to current materials. Hence, there is a continuing need for tailored rhamnolipid mixtures of congeners that can provide improved efficacy and consumer experience.

### SUMMARY OF THE INVENTION

A composition comprising rhamnolipid compositions, said rhamnolipid compositions comprising (a) and (b), and said composition further comprising (c), wherein (a), (b), and (c) are as follows:

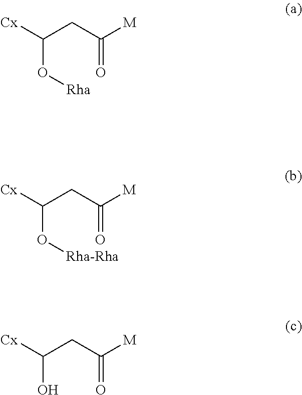

wherein Rha is rhamnose;
wherein each Cx is independently selected from an alkyl, aryl, heteroalkyl, heteroaryl, unsaturated alkenyl, and unsaturated heteroalkenyl;
wherein each Cx independently has a carbon chain length from 4 to 22;
wherein each M is independently selected from OH; O−X+ wherein X+ is a cation; OR1; alkyl, heteroalkyl; aryl; heteroaryl; hetero arylalkyl; arylalkyl; and tauryl;
wherein R1 is selected from an alkyl, branched alkyl, and cyclic alkyl;
and all possible stereoisomers thereof, and all combinations thereof; and
wherein (a) or (b) is at least about 25 wt % or greater of all rhamnolipids present in the compositions.

### BRIEF DESCRIPTION OF THE FIGURES

For a more complete understanding of the disclosure, reference should be made to the following detailed description and Figures.

FIG. 1 shows exemplary rhamnolipid structures.

FIG. 2 shows an exemplary route for synthesizing rhamno-mono-lipids.

FIG. 3 is a graph showing the impact of the temperature and time of the hydrolysis reaction on the color of inventive compositions.

FIG. 4 is a graph showing the impact of the temperature and time of the hydrolysis reaction on the color of inventive compositions.

FIG. 5 is a graph of the oven program for the test method for Beta Hydroxy Fatty Acid Quantitation by GC-FID.

### DETAILED DESCRIPTION OF THE INVENTION

There is interest in non-traditional surfactants, such as glycolipid biosurfactants, as many palm- and petroleum-based surfactants suffer from sustainability, environmental, and socioeconomic challenges. Glycolipids consist of a diverse group of naturally occurring surfactant molecules with a range of structures (made up of a sugar polar group and a lipid group). The two main commercial classes of glycolipids are rhamnolipids (produced via bacterial plus fermentation) and sophorolipids (produced via yeast fermentation of mixed oil and sugar feed). In addition to being seen as environmentally friendly chemicals and enabling green credentialling, these materials have many other potential benefits such as mildness, moisturization, and cleaning effectiveness.

Many current commercial glycolipids have poor performance and high costs compared to current surfactants. Knowing the structures of certain high-performing rhamnolipids, the present inventors sought to produce optimized glycolipids. The inventors hypothesized that changing the molecular structure via simplification of the surfactant headgroup and elongation to a single chain length (making them more structurally similar to typical surfactants) would increase surfactancy. One of the production strategies involved a short-term, semi-synthetic fermentation approach, in which commercial rhamnolipids were leveraged as the feedstock and hydrolyzed to produce simplified mono-lipid glycolipid congener structures.

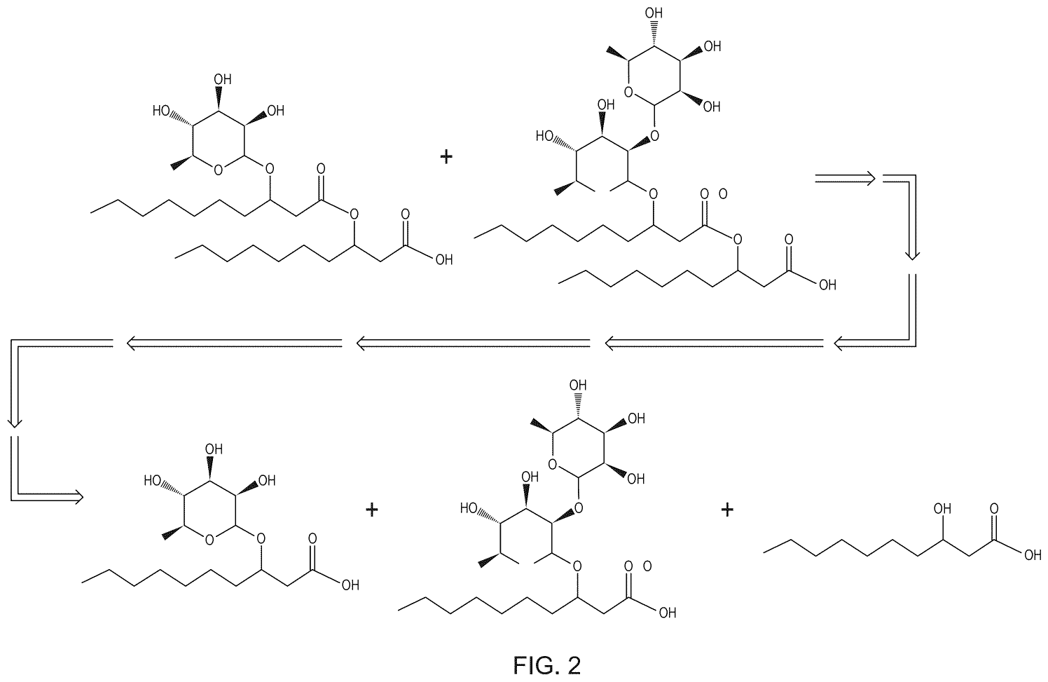

Commercial Rheance® One rhamnolipids (Rheance 1, made by Evonik Industries AG, Essen, Germany) were used to demonstrate nearly complete hydrolysis from a monorhamnodilipid plus dirhamnodilipid species to a monorhamnomonolipid plus dirhamnomonolipid species, as exemplified in FIG. 2.

The present inventors have discovered that similar hydrolysis can be done on other commercially available rhamnolipids, such as those sold by Evonik Industries AG, Essen, Germany, by BioReNuva, Austin, TX, USA, and by Wanhua Chemical Group Co., Ltd., Yantai, China. While not necessarily limited to creation by the hydrolysis process, these inventive rhamnolipid mixtures offer commercial and consumer benefits.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

When used in the context of a chemical group: “hydrogen” means-H; “hydroxy” means —OH; “oxo” means =O; “carbonyl” means —C(═O)—; “carboxy” and “carboxylate” mean —C(═O)OH (also written as —COOH or —CO₂H) or a deprotonated form thereof; “amino” means —NH₂; “hydroxyamino” means —NHOH; “nitro” means —NO₂; “imino” means =NH; “amine oxide” means N+O where N has three covalent bonds to atoms other than O; “hydroxamic” or “hydroxamate” means —C(O)NHOH or a deprotonated form thereof.

The term “cation” refers to an atom, molecule, or a chemical group with a net positive charge including single and multiple charged species. Cations can be individual atoms such as metals, non-limiting examples include Na⁺ or Ca⁺², individual molecules, non-limiting examples include (CH₃)₄N⁺, or a chemical group, non-limiting examples include N(CH₃)₃⁺. The term “amine cation” refers to a particular molecular cation, of the form NR₄⁺ where the four substituting R moieties can be independently selected from H and alkyl, non-limiting examples include NH₄⁺ (ammonium), CH₃NH₃⁺ (methylammonium), CH₃CH₂NH₃⁺ (ethylammonium), (CH₃)₂NH₂⁺ (dimethylammonium), (CH₃)₂NH⁺ (trimethyl ammonium), and (CH₃)₄N⁺ (tetramethylammonium). In some embodiments, a cation may be selected from Na+, K+, Li+, Cs+, +NH₃R2; +NH₂R2R3; +NHR2R3R4, +NR2R3R4R5 wherein R2, R3, R4, and R5 are each independently selected from an alkyl, branched alkyl, and cyclic alkyl.

The term “anion” refers to an atom, molecule, or chemical group with a net negative charge including single and multiply charged species. Anions can be individual atoms, for example but not limited to halides F, Cl, Br, individual molecules, non-limiting examples include CO₃⁻², H₂PO₄⁻, HPO₄⁻², PO₄⁻³, HSO₄⁻, SO₄⁻², or a chemical group, non-limiting examples include sulfate, phosphate, sulfonate, phosphonate, phosphinate, sulfonate, mercapto, carboxylate, amine oxide, hydroxamate and hydroxyl amino. Deprotonated forms of previously defined chemical groups are considered anionic groups if the removal of the proton results in a net negative charge. In solutions, chemical groups are capable of losing a proton and become anionic as a function of pH according to the Henderson-Hasselbach equation (pH=pKa+log₁₀([A⁻]/[HA]; where [HA] is the molar concentration of an undissociated acid and [A⁻] is the molar concentration of this acid's conjugate base). When the pH of the solution equals the pKa value of functional group, 50% of the functional group will be anionic, while the remaining 50% will have a proton. Typically, a functional group in solution can be considered anionic if the pH is at or above the pKa of the functional group.

The term “salt” or “salts” refers to the charge neutral combination of one or more anions and cations. For example, when R is denoted as a salt for the carboxylate group, —COOR, it is understood that the carboxylate (—COO—) is an anion with a negative charge −1, and that the R is a cation with a positive charge of +1 to form a charge neutral entity with one anion of charge −1, or R is a cation with a positive charge of +2 to form a charge neutral entity with two anions both of −1 charge.

The term “saturated” as used herein means the chemical compound or group so modified has no carbon-carbon double and no carbon-carbon triple bonds, except as noted below. In the case of substituted versions of saturated chemical groups, one or more carbon oxygen double bond or a carbon nitrogen double bond may be present. When such a bond is present, then carbon-carbon double bonds that may occur as part of keto-enol tautomerism or imine/enamine tautomerism are not precluded.

The term “aliphatic” when used without the “substituted” modifier signifies that the chemical compound/group so modified is an acyclic or cyclic, but non-aromatic hydrocarbon chemical compound or group. In aliphatic chemical compounds/groups, the carbon atoms can be joined together in straight chains, branched chains, or non-aromatic rings (alicyclic). Aliphatic chemical compounds/groups can be saturated, that is joined by single bonds (alkanes/alkyl), or unsaturated, with one or more double bonds (alkenes/alkenyl), or with one or more triple bonds (alkynes/alkynyl).

The term “alkyl” when used without the “substituted” modifier refers to a monovalent saturated aliphatic group with a carbon atom as the point of attachment, a linear or branched, cyclo, cyclic, or acyclic structure, and no atoms other than carbon and hydrogen. Thus, as used herein cycloalkyl is a subset of alkyl, with the carbon atom that forms the point of attachment also being a member of one or more non-aromatic ring structures wherein the cycloalkyl group consists of no atoms other than carbon and hydrogen. As used herein, the term does not preclude the presence of one or more alkyl groups (carbon number limitation permitting) attached to the ring or ring system. The groups —CH₃(Me), —CH₂CH₃(Et), —CH₂CH₂CH₃(n-Pr or propyl), —CH(CH₃)₂ (i-Pr, 'Pr, or isopropyl), —CH(CH₂)₂ (cyclopropyl), —CH₂CH₂CH₂CH₃(n-Bu), —CH(CH₃) CH₂CH₃(sec-butyl), —CH₂CH(CH₃)₂ (isobutyl), —C(CH₃)₃(tertbutyl, t-butyl, t-Bu, or tBu), —CH₂C(CH₃)₃(neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: =CH₂, ═CH(CH₂CH₃), and =C(CH₃)₂. An “alkane” refers to the compound H-R, wherein R is alkyl as this term is defined above.

When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, —S(O)₂NH₂, —P(O)(OH)₂, —P(O)(OH)OP(O)(OH)₂, —OP(O)(OH)₂, —OP(O)(OH)OP(O)(OH)₂, —S(O)₂(OH), or —OS(O)₂(OH). The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, —CH₂CH₂Cl, —CH₂P(O)(OH)₂, —CH₂P(O)(OH) OP(O)(OH)₂, —CH₂S(O)₂(OH), and —CH₂OS(O)₂(OH). The term “haloalkyl” is a subset of substituted alkyl, in which one or more hydrogen atoms has been substituted with a halo group and no other atoms aside from carbon, hydrogen and halogen are present. The group, —CH₂Cl is a non-limiting example of a haloalkyl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups.

The term “alkenyl” when used without the “substituted” modifier refers to a monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —C(CH₃)=CH₂ (methyl-vinyl), —CH═CHCH₃, —CH—CHCH₂CH₃, —CH₂CH—CH₂ (allyl), —CH₂CH—CHCH₃, and —CH═CHCH═CH₂. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, >C═CH₂ (vinylidine), —CH═CH—, —CH═C(CH₃) CH₂—, and —CH═CHCH₂—, are non-limiting examples of alkenediyl groups. It is noted that while the alkenediyl group is aliphatic, once connected at both ends, this group is not precluded from forming part of an aromatic structure. The terms “alkene” or “olefin” are synonymous and refer to a compound having the formula H-R, wherein R is alkenyl as this term is defined above.

The term “aryl” when used without the “substituted” modifier refers to a functional group derived from a simple aromatic ring compound where the point of attachment is a carbon atom on the aromatic ring. An aromatic ring is a hydrocarbon that has a cyclic structure and a delocalized electron system. An aryl group is formed by removing one hydrogen atom from the ring. The name of the aryl group is based on the name of the aromatic ring with the -yl suffix, such as phenyl, naphthyl, indolyl, etc. Aryl groups may be substituted with alkyl and/or heteroalkyl chains and may have one or more heteroatoms within the aryl ring.

The term “arylalkyl” refers to an aryl group attached to an alkanediyl group where the point of attachment is on the alkanediyl group.

Tauryl/taurate is defined as aminoethyl sulphonyl.

Hetero is defined as an atom other than carbon, including but not exclusive to, nitrogen (N), oxygen (O), or sulfur(S). The heteroatom may be attached in a linear or branched alkyl chain or also may be attached to a non-aromatic or aromatic ring, either as part of the ring, or adjacent to it as a substituent. There may be more than one heteroatom in the alkyl chain or ring.

Unsaturated is defined as a hydrocarbon chain possessing, but not limited, at least one carbon-carbon double bond (C═C). The unsaturated chain may possess one double bond (alkenyl), two double bonds (dienyl), multiple double bonds (polyenyl), and/or a carbon-carbon triple bond (acetylenic). The unsaturated bonds may be adjacent (conjugated) relative to each other or separated by additional carbon atoms in the chain.

A “monomer molecule” is defined by the International Union of Pure and Applied Chemistry (IUPAC) as “A molecule which can undergo polymerization thereby contributing constitutional units to the essential structure of a macromolecule.” A polymer is a macromolecule.

“Treat” or “treating” as used in reference to a composition, means to add or apply a material to the composition.

“About” modifies a particular value by referring to a range of plus or minus 20% or less of the stated value (e.g., plus or minus 15% or less, 10% or less, or even 5% or less, or even 1% or less).

“Apply” or “application,” as used in reference to a composition, means to apply or spread the composition onto a human keratinous surface such as the skin or hair.

“Personal care composition” is meant a product, which in the ordinary course of usage is applied to or contacted with a body surface to provide a beneficial effect. Body surface includes skin, for example dermal or mucosal; body surface also includes structures associated with the body surface for example hair, teeth, or nails. Examples of personal care compositions include a product applied to a human body for improving appearance, cleansing, and odor control or general aesthetics. Non-limiting examples of personal care compositions include oral care compositions, such as, dentifrice, mouth rinse, mousse, foam, mouth spray, lozenge, chewable tablet, chewing gum, tooth whitening strips, floss and floss coatings, breath freshening dissolvable strips, denture care product, denture adhesive product; after shave gels and creams, pre-shave preparations, shaving gels, creams, or foams, moisturizers and lotions; cough and cold compositions, gels, gel caps, and throat sprays; leave-on skin lotions and creams, shampoos, body washes, body rubs, such as Vicks VapoRub; hair conditioners, hair dyeing and bleaching compositions, mousses, shower gels, bar soaps, antiperspirants, deodorants, depilatories, lipsticks, foundations, mascara, sunless tanners and sunscreen lotions; feminine care compositions, such as lotions and lotion compositions directed towards absorbent articles; baby care compositions directed towards absorbent or disposable articles; and oral cleaning compositions for animals, such as dogs and cats. Further non-limiting examples include hand soaps, hand sanitizers, body washes, shower gels, shampoos, body lotions, feminine care products, foot care products, deodorants, pet care products and combinations thereof. Further non-limiting examples include a wipe product suitable for personal care use and household cleaning; a toilet tissue; a towel for hand drying, household drying and household cleaning; a facial tissue; a skin care composition; a first aid or surgical antiseptic; a diaper; a feminine napkin; and combinations thereof.

The term “detergent composition” refers to a composition or formulation designed for cleaning soiled surfaces. Such compositions include but are not limited to, dishwashing compositions, laundry detergent compositions, fabric softening compositions, fabric enhancing compositions, fabric freshening compositions, laundry pre-wash, laundry pretreat, laundry additives, spray products, dry cleaning agent or composition, laundry rinse additive, wash additive, post-rinse fabric treatment, ironing aid, hard surface cleaning compositions, unit dose formulation, delayed delivery formulation, detergent contained on or in a porous substrate or nonwoven sheet, and other suitable forms that may be apparent to one skilled in the art in view of the teachings herein. Such compositions may be used as a pre-cleaning treatment, a post-cleaning treatment, or may be added during the rinse or wash cycle of the cleaning process. The detergent compositions may have a form selected from liquid, powder, single-phase or multi-phase unit dose or pouch form, tablet, gel, paste, bar, or flake. Preferably the composition is for manual washing. The detergent composition of the present invention may be a dishwashing detergent. The composition may be in the form of a liquid. Further non-limiting examples include hard surface cleaners, deodorizers, fabric care compositions, fabric cleaning compositions, manual dish detergents, automatic dish detergents, floor waxes, kitchen cleaners, bathroom cleaners and combinations thereof.

The term “liquid detergent composition” refers to a liquid detergent composition which is fluid, and preferably capable of wetting and cleaning a fabric, e.g., clothing in a domestic washing machine. As used herein, “laundry detergent composition” refers to compositions suitable for washing clothes. The liquid laundry detergent composition preferably has a density in the range from 0.9 to 1.3 grams per cubic centimeter, more specifically from 1.00 to 1.10 grams per cubic centimeter, excluding any solid additives but including any bubbles, if present. Aqueous liquid laundry detergent compositions are preferred. For such aqueous liquid laundry detergent compositions, the water content can be present at a level of from 5% to 99%, preferably from 15% to 90%, more preferably from 25% to 80% by weight of the liquid detergent composition. The pH range of the detergent composition may be from 7.5 to 9.5, preferably from pH 8 to 9.

“Cleansing composition” refers to a personal care composition or product intended for use in cleaning a bodily surface such as skin or hair. Some non-limiting examples of cleansing compositions are shampoos, conditioners, conditioning shampoos, shower gels, liquid hand cleansers, facial cleansers, unit-dose cleansers in the form of soluble fibers, and the like.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration and food additives.

“Gel network phase” or “dispersed gel network phase” refers to a lamellar or vesicular solid crystalline phase that includes at least one fatty alcohol, at least one gel network surfactant, and a liquid carrier. The lamellar or vesicular phase can be formed of alternating layers with one layer including the fatty alcohol and the gel network surfactant and the other layer formed of the liquid carrier.

“Solid crystalline” refers to the crystalline structure of the lamellar or vesicular phase at ambient temperatures caused by the phase being below its melt transition temperature. For example, the melt transition temperature of the lamellar or vesicular phase may be about 30° C. or more (i.e., slightly above about room temperature). The melt transition temperature can be measured through differential scanning calorimetry, which is conventional measurement method known to those skilled in the art.

“Suitable for application to human hair” means that the personal care composition or components thereof, are acceptable for use in contact with human hair and the scalp and skin without undue toxicity, incompatibility, instability, allergic response, and the like.

“Substantially free of” means a composition or ingredient comprises less than 3% of a subject material, by weight of the composition or ingredient (e.g., less than 2%, less than 1% or even less than 0.5%). “Free of” means a composition or ingredient contains 0% of a subject material.

“Sulfated surfactants” means surfactants that contain a sulfate moiety. Some non-limiting examples of sulfated surfactants are sodium lauryl sulfate, sodium laureth sulfate, ammonium lauryl sulfate, and ammonium laureth sulfate. “Sulfate-free surfactant” refers to a surfactant that has no sulfate moieties.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### Rhamnolipid Surfactant

Rhamnose (Rha, Rham), formula illustrated below, is a naturally occurring deoxy sugar. It can be classified as either a methyl-pentose or a 6-deoxy-hexose.

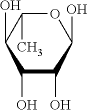

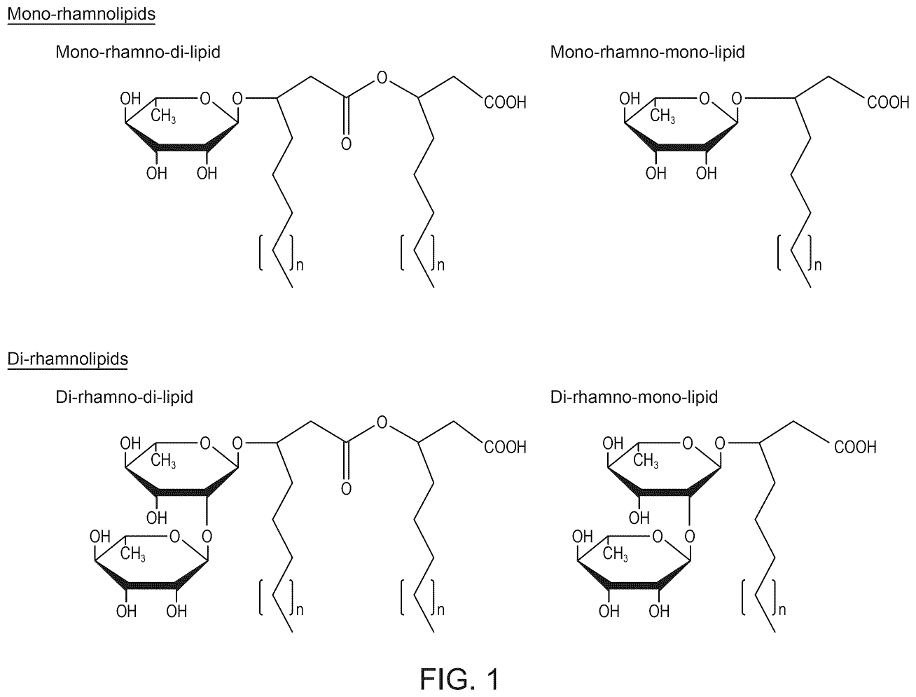

Rhamnolipids are a class of glycolipid that may be used as bacterial-derived surfactants. They have a glycosyl head group, in this case a rhamnose moiety, and an acid fatty tail. There are two main classes of rhamnolipids: mono-rhamnolipids and di-rhamnolipids, which consist of one or two rhamnose groups respectively. And then mono-rhamnolipids and di-rhamnolipids may each have either one or two lipids, creating, for example, the molecules shown in FIG. 1.

The compositions described herein include one or more rhamnolipid biosurfactants. The rhamnolipid surfactants herein may be produced by microorganisms (e.g., Pseudomonas aeruginosa, Pseudomonas putida, Pseudomonas chlororaphis). The rhamnolipid surfactant(s) may provide a cleaning benefit due to their amphiphilic nature, which allows the surfactants to break up, and form micelles around, oil and other contaminants. The “entrapped” contaminant can then be rinsed off more easily with water. A description of various types of rhamnolipids is disclosed in EP2410039. Methods of making, extracting, and blending naturally produced rhamnolipids are known in the art.

The present inventors have discovered that performing a base hydrolysis of commercially-available rhamnolipids produces a mixture of monolipid rhamnolipid species. These mixtures may be found to improve a formulated material's surfactancy. In the present invention, for example, a semi-synthetic fermentation approach may be used, in which commercial rhamnolipids are leveraged as the feedstock and hydrolyzed to produce the simplified monolipid glycolipid structures.

For example, commercially available Rheance One rhamnolipids, sold by Evonik, were nearly completely hydrolyzed from a mono-rhamno-dilipid and di-rhamno-dilipid species to a mono-rhamno-monolipid and di-rhamno-monolipid species, as shown in FIG. 2.

The composition that results from the hydrolysis is a composition comprising rhamnolipid compositions, said rhamnolipid compositions comprising (a) and (b), and said composition further comprising (c), wherein (a), (b), and (c) are as follows:

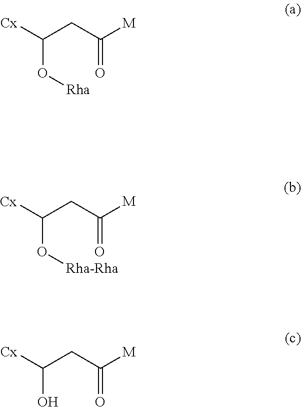

wherein Rha is rhamnose;
wherein each Cx is independently selected from an alkyl, aryl, heteroalkyl, heteroaryl, unsaturated alkenyl, and unsaturated heteroalkenyl;
wherein each Cx independently has a carbon chain length from 4 to 22;
wherein each M is independently selected from OH; O−X+ wherein X+ is a cation; OR1; alkyl, heteroalkyl; aryl; heteroaryl; hetero arylalkyl; arylalkyl; and tauryl;
wherein R1 is selected from an alkyl, branched alkyl, and cyclic alkyl;
and all possible stereoisomers thereof, and all combinations thereof;
   wherein (a) or (b) is at least about 25 wt % of all rhamnolipids present in the compositions.

For any given embodiment, Cx, M, and R1 may be any combination thereof.

The individual (a), (b), and (c) components may each have many variations, and any stereoisomer of any (a), (b), or (c) may be included as a possibility. In addition, any combination of (a), (b), and (c), may be an inventive composition, as long as each of (a), (b), and (c) fall within the generalized formula for its respective designation.

The inventive compositions resulting from the hydrolysis may comprise rhamnolipids in which at least about 25% by weight of all the rhamnolipids are monolipids. In some embodiments, the weight % of all rhamnolipids in an inventive composition that are monolipids may be at least about 15%, at least about 20%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or at least about 100%. In some embodiments, the weight % of all rhamnolipids in an inventive composition that are monolipids may be from about 25%, about 30%, about 35%, about 40%, about 45%, about 50%, about 55%, about 60%, about 65%, about 70%, about 75%, about 80%, about 85%, about 90%, about 95%, to about 40%, about 45%, about 50%, about 55%, about 60%, about 65%, about 70%, about 75%, about 80%, about 85%, about 90%, about 95%, about 96%, about 97%, about 98%, about 99%, or about 100%, with any combination of these percentages herein.

In some embodiments, the inventive composition may comprise rhamnolipids in which a monorhamnomonolipid or composition (a) comprises at least about 20%, about 30%, about 40%, about 50%, about 60%, about 70%, about 80%, about 90%, about 95%, about 99%, or about 100% by weight of all the rhamnolipids, or in some embodiments from about 20% to about 100%, or about 25% to about 95%, or any combination therebetween, by weight of all rhamnolipids. In other embodiments, the inventive composition may comprise rhamnolipids in which a dirhamnomonolipid or composition (b) comprises at least about 20%, about 30%, about 40%, about 50%, about 60%, about 70%, about 80%, about 90%, about 95%, about 99%, or about 100% by weight of all the rhamnolipids, or in some embodiments from about 20% to about 100%, or about 25% to about 95%, or any combination therebetween, by weight of all rhamnolipids.

In some embodiments, the starting material that is hydrolyzed may be completely converted, that is, 100% converted, such that the inventive composition may consist of or consist essentially of (a), (b), and (c). In some embodiments, the inventive composition may comprise from about 50% to about 100% of (a), (b), and (c), or any combination therebetween. The remainder of the inventive composition may comprise or consist of any of the components from the original unhydrolyzed starting material. For example, the inventive composition may comprise some rhamnodilipids, including some monorhamnodilipids and/or some dirhamnodilipids. In some embodiments, the rhamnodilipids may be at most about 10%, at most about 15%, at most about 20%, at most about 25% by weight of all rhamnolipids. In some embodiments, monorhamnodilipids may be at most about 10%, at most about 15%, at most about 20%, at most about 25% by weight of all rhamnolipids. In some embodiments, dirhamnodilipids may be at most about 10%, at most about 15%, at most about 20%, at most about 25% by weight of all rhamnolipids. In one embodiment, the dirhamnodilipids are at most about 25 wt % of all rhamnolipid compositions present in the composition.

For the inventive compositions, R, R1, R2, R3, R4, and R5 may be independently defined as alkyl, branched alkyl, and cyclic alkyl, including methyl, ethyl, propyl, butyl, isopropyl, isobutyl, cyclopropyl, cyclobutyl, cyclohexyl, and alkenyl. In addition, the groups-CH₃ (Me), —CH₂CH₃ (Et), —CH₂CH₂CH₃ (n-Pr or propyl), —CH(CH₃)₂ (i-Pr, 'Pr, or isopropyl), —CH(CH₂)₂ (cyclopropyl), —CH₂CH₂CH₂CH₃ (n-Bu), —CH(CH₃) CH₂CH₃ (sec-butyl), —CH₂CH(CH₃)₂ (isobutyl), —C(CH₃)₃ (tertbutyl, t-butyl, t-Bu, or tBu), —CH₂C(CH₃)₃ (neo-pentyl), cyclobutyl, cyclopentyl, cyclohexyl, and cyclohexylmethyl are non-limiting examples of alkyl groups.

Cx may be independently defined as or selected from an alkyl, aryl, heteroalkyl, heteroaryl, unsaturated alkyl, and unsaturated heteroalkyl, wherein each Cx independently has a carbon chain length from 4 to 22, or in some embodiments from 5 to 13, or in some embodiments the carbon chain length may be 10 or may be 12; more specifically, Cx can be the C-3 carbon (C5-C13) chain pendant to the glyceride carboxy terminus as shown in Table Q.

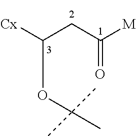

TABLE Q

| Cx | Name |
|---|---|
| 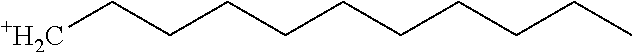 | Undecyl (C11) |
| 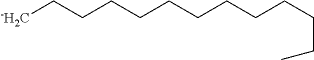 | Tridecyl (C13) |
| 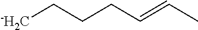 | Heptenyl (C7:1) |
| 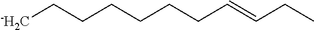 | Undecenyl (C11:1) |
| 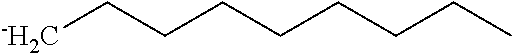 | Nonyl (C9) |
| 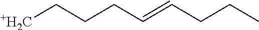 | Nonenyl (C9:1) |
| 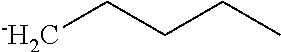 | Pentyl (C5) |
| 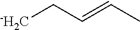 | Pentenyl (C5:1) |
| 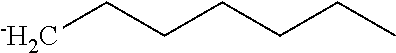 | Heptyl (C7) |
| 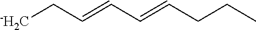 | Nonadienyl (9:2) |

### Experimental Procedures for Rhamnolipid Hydrolysis

### Example A

### Synthesis of Rheance One Hydrolysate Sodium Salt

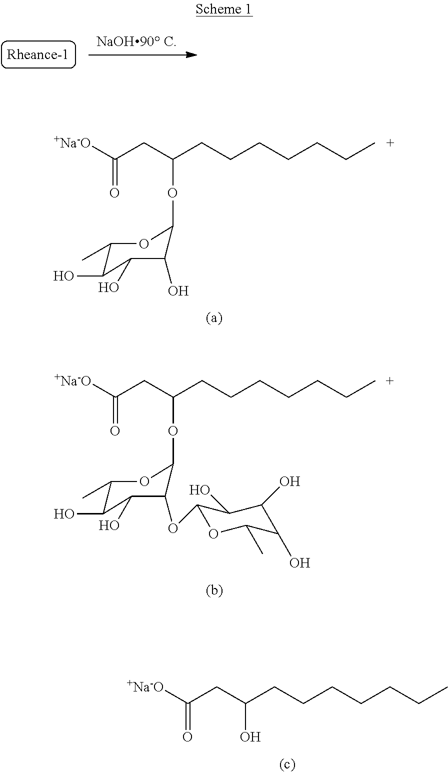

In a 2 L, 3-neck round-bottomed flask equipped with reflux condenser with N₂ inlet regulator, Caframo RZR-2000 Overhead Stirrer, and J-Kem Scientific Temperature Controller with probe was charged with a 500 grams weight solution of Rheance One, (Evonik, 50% weight in H₂O, 250 g, Mw˜683, 0.37 mol), followed by NaOH pellets (CAS 1310-73-2, Mw=40, 30 grams, 0.74 mol). The reaction was heated to 90° C. and stirred for 3 hours. The solution darkened over this time. The reaction was cooled to room temperature and was then lyophilized to remove water. (Virtis Model #50L Virtual XL-70, 130 mTorr, 72 hrs, 0° C.-room temperature shelf temperature). The result was a reddish, tacky solid (230 grams), predominately containing (b) and (c) in greater than 25%, which was used without further purification.

By utilizing standard analytical methods, the following materials were identified in the hydrolysate reaction after completion for Example A (Table 1).

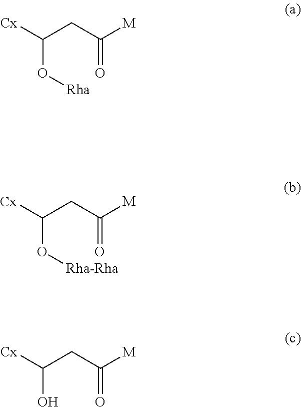

TABLE 1

| Entry | Structure (a) | Structure (b) |
|---|---|---|
| 1 | | |
| 2 | | |
| 3 | | |
| 4 | | |
| 5 | | |
| 6 | | 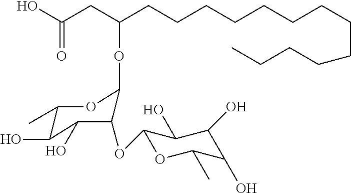 [SMILES: CCCCCCCCCCCCCC(CC(=O)O)OC1OC(C)C(O)C(O)C1OC1OC(C)C(O)C(O)C1O] |
| 7 | | 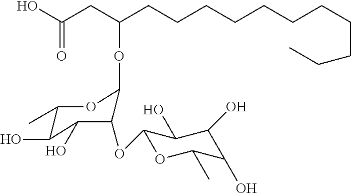 [SMILES: CCCCCCCCCCCC(CC(=O)O)OC1OC(C)C(O)C(O)C1OC1OC(C)C(O)C(O)C1O] |
| 8 | | |
| 9 | | 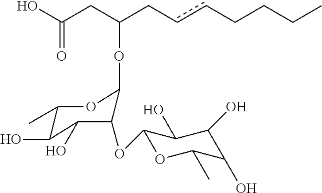 |
| 10 | | |
| 11 | 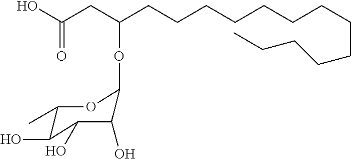 [SMILES: CCCCCCCCCCCCCC(CC(=O)O)OC1OC(C)C(O)C(O)C1O] | |
| 12 | 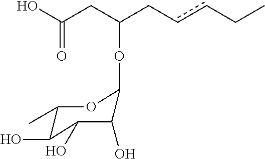 | |
| 13 | | |
| 14 | | 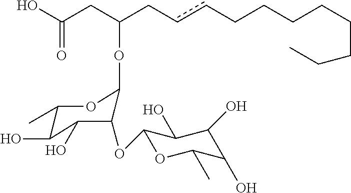 |
| 15 | | 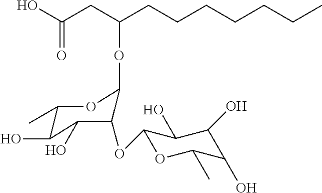 [SMILES: CCCCCCCC(CC(=O)O)OC1OC(C)C(O)C(O)C1OC1OC(C)C(O)C(O)C1O] |
| 16 | 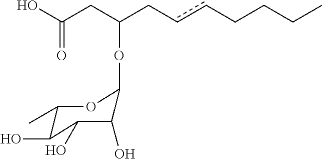 | |
| 17 | | 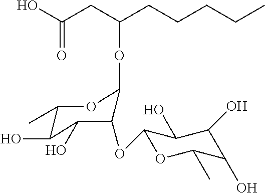 [SMILES: CCCCCC(CC(=O)O)OC1OC(C)C(O)C(O)C1OC1OC(C)C(O)C(O)C1O] |
| 18 | | 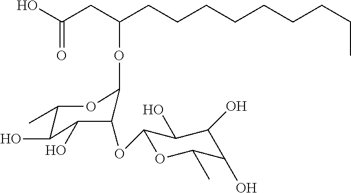 [SMILES: CCCCCCCCCC(CC(=O)O)OC1OC(C)C(O)C(O)C1OC1OC(C)C(O)C(O)C1O] |
| 19 | 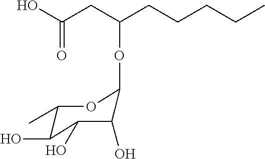 [SMILES: CCCCCC(CC(=O)O)OC1OC(C)C(O)C(O)C1O] | |
| 20 | 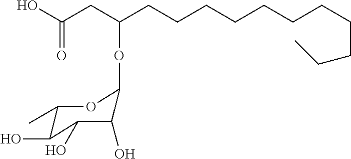 [SMILES: CCCCCCCCCCCC(CC(=O)O)OC1OC(C)C(O)C(O)C1O] | |
| 21 | | |
| 22 | 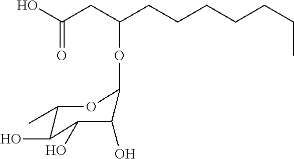 [SMILES: CCCCCCCC(CC(=O)O)OC1OC(C)C(O)C(O)C1O] | |
| 23 | 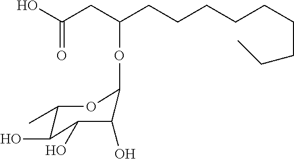 [SMILES: CCCCCCCCCC(CC(=O)O)OC1OC(C)C(O)C(O)C1O] | |
| 24 | 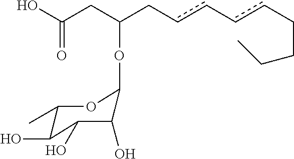 | |
| Entry | Structure (c) | Chemical Name |
| 1 | 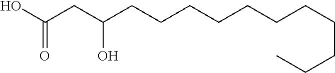 [SMILES: CCCCCCCCCCCC(O)CC(=O)O] | 3-hydroxytetradecanoic acid |
| | 3-hydroxytetradecanoic acid | |
| 2 | 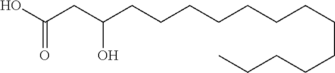 [SMILES: CCCCCCCCCCCCCC(O)CC(=O)O] | 3-hydroxyhexadecanoic acid |
| 3 | 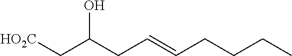 | 3-hydroxydecenoic acid |
| 4 | 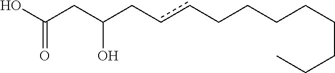 | 3-hydroxytetradecenoic acid |
| 5 | 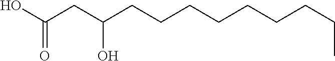 | 3-hydroxydodecanoic acid |
| 6 | | 3-(((2R,3R,4R,5R,6S)-4,5- |
| | | dihydroxy-6-methyl-3- |
| | | (((2R,3S,4S,5S,6S)-3,4,5- |
| | | trihydroxy-6-methyltetrahydro- |
| | | 2H-pyran-2-yl)oxy)tetrahydro- |
| | | 2H-pyran-2-yl)oxy)hexadecanoic acid |
| 7 | | 3-(((2R,3R,4R,5R,6S)-4,5- |
| | | dihydroxy-6-methyl-3- |
| | | (((2R,3S,4S,5S,6S)-3,4,5- |
| | | trihydroxy-6-methyltetrahydro- |
| | | 2H-pyran-2-yl)oxy)tetrahydro- |
| | | 2H-pyran-2-yl)oxy)tetradecanoic |
| | | acid |
| 8 | 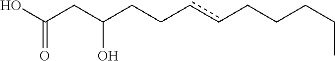 | 3-hydroxydodecenoic acid |
| 9 | | 3-(((2R,3R,4R,5R,6S)-4,5- |
| | | dihydroxy-6-methyl-3- |
| | | (((2R,3S,4S,5S,6S)-3,4,5- |
| | | trihydroxy-6-methyltetrahydro- |
| | | 2H-pyran-2-yl)oxy)tetrahydro- |
| | | 2H-pyran-2-yl)oxy)decenoic acid |
| 10 | 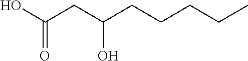 | 3-hydroxyoctanoic acid |
| 11 | | 3-(((2R,3R,4R,5R,6S)-3,4,5- |
| | | trihydroxy-6-methyltetrahydro- |
| | | 2H-pyran-2-yl)oxy)hexadecanoic acid |
| 12 | | 3-(((2R,3R,5R,6S)-3,4,5- |
| | | trihydroxy-6-methyltetrahydro- |
| | | 2H-pyran-2-yl)oxy)octenoic acid |
| 13 | 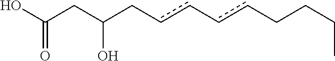 | 3-hydroxydodec-dienoic acid |
| 14 | | 3-(((2R,3R,4R,5R,6S)-4,5- |
| | | dihydroxy-6-methyl-3- |
| | | (((2R,3S,4S,5S,6S)-3,4,5- |
| | | trihydroxy-6-methyltetrahydro- |
| | | 2H-pyran-2-yl)oxy)tetrahydro- |
| | | 2H-pyran-2-yl)oxy)tetradecenoic acid |
| 15 | | 3-(((2R,3R,4R,5R,6S)-4,5- |
| | | dihydroxy-6-methyl-3- |
| | | (((2R,3S,4S,5S,6S)-3,4,5- |
| | | trihydroxy-6-methyltetrahydro- |
| | | 2H-pyran-2-yl)oxy)tetrahydro- |
| | | 2H-pyran-2-yl)oxy)decanoic acid |
| 16 | | 3-(((2R,3R,4R,5R,6S)-3,4,5- |
| | | trihydroxy-6-methyltetrahydro- |
| | | 2H-pyran-2-yl)oxy)decenoic acid |
| 17 | | 3-(((2R,3R,4R,5R,6S)-4,5- |
| | | dihydroxy-6-methyl-3- |
| | | (((2R,3S,4S,5S,6S)-3,4,5- |
| | | trihydroxy-6-methyltetrahydro- |
| | | 2H-pyran-2-yl)oxy)tetrahydro- |
| | | 2H-pyran-2-yl)oxy)octanoic acid |
| 18 | | 3-(((2R,3R,4R,5R,6S)-4,5- |
| | | dihydroxy-6-methyl-3- |
| | | (((2R,3S,4S,5S,6S)-3,4,5- |
| | | trihydroxy-6-methyltetrahydro- |
| | | 2H-pyran-2-yl)oxy)tetrahydro- |
| | | 2H-pyran-2-yl)oxy)dodecanoic acid |
| 19 | | 3-(((2R,3R,4R,5R,6S)-3,4,5- |
| | | trihydroxy-6-methyltetrahydro- |
| | | 2H-pyran-2-yl)oxy)octanoic acid |
| 20 | | 3-(((2R,3R,4R,5R,6S)-3,4,5- |
| | | trihydroxy-6-methyltetrahydro- |
| | | 2H-pyran-2-yl)oxy)tetradecanoic acid |
| 21 | | 3-hydroxydecaneoic acid |
| 22 | | 3-(((2R,3R,4R,5R,6S)-3,4,5- |
| | | trihydroxy-6-methyltetrahydro- |
| | | 2H-pyran-2-yl)oxy)decanoic acid |
| 23 | | 3-(((2R,3R,4R,5R,6S)-3,4,5- |
| | | trihydroxy-6-methyltetrahydro- |
| | | 2H-pyran-2-yl)oxy)dodecanoic acid |
| 24 | | 3-(((2R,3R,4R,5R,6S)-3,4,5- |
| | | trihydroxy-6-methyltetrahydro- |
| | | 2H-pyran-2-yl)oxy)dodecen- |
| | | dienoic acid |

Both the starting commercial material, Rheance One, and the hydrolyzed inventive material of Example A Scheme 1 were subjected to mass spectroscopy. Table 1.2 has the mass spectroscopy data of the Rheance One before and after hydrolysis. A rhamnose group is represented as (Rha) and a lipid is abbreviated as Cn or Cn: m (where n=number of carbon atoms; m=number of unsaturation). ND=not detected. % Difference of molecule post hydrolysis=(AUC post hydrolysis)/(AUC before hydrolysis)*100.

After hydrolysis, the rhamno-mono-lipid species detection increased by 184-1092%. In some cases, new rhamno-mono-lipid molecules were detected. The rhamno-di-lipid were completely hydrolyzed (not detected) or detected at very low and insignificant amounts, i.e., 0.6-24%. This shows that the rhamnolipids in the inventive compositions have a high amount of monolipids.

TABLE 1.2

| | | | | % Differ- |
|---|---|---|---|---|
| | | | | ence |
| | | | | of |
| | | AUC | AUC | molecule |
| Rhamnolipid | | (Pre- | (Post- | post |
| Class | Molecule | hydrolysis) | hydrolysis) | hydrolysis |
| Mono- | Rha-C8 | 1063249 | 3813199 | 358.636 |
| rhamno- | Rha-C10 | ND | 8606355 | New |
| mono-lipid | | | | molecule |
| | Rha-C12 | 537716 | 993329 | 184.731 |
| | Rha-C12:1 | 15662 | ND | ND |
| | Rha-C14:1 | ND | 161875 | New |
| | | | | molecule |
| Di-rhamno- | RhaRha-C8 | 24485006 | 100543784 | 410.634 |
| mono-lipid | RhaRha-C10 | 173099952 | 807753664 | 466.640 |
| | RhaRha-C10:1 | 255899 | 2795532 | 1092.436 |
| | RhaRha-C12 | 15592295 | 101665328 | 652.023 |
| | RhaRha-C12:1 | ND | 14508326 | New |
| | | | | Molecule |
| | RhaRha-C14 | 700558 | 5241753 | 748.225 |
| | RhaRha-C14:1 | 2420763 | 15934095 | 658.226 |
| | RhaRha-C16 | 62651 | 540257 | 862.328 |
| Mono- | Rha-C8C8 | 4988452 | ND | ND |
| rhamno-di- | Rha-C8C10 | 125784560 | 727010 | 0.578 |
| lipid | Rha-C8C10:1 | 51561 | ND | ND |
| | Rha-C8C12 | 422537 | ND | ND |
| | Rha-C9C10 | 400385 | ND | ND |
| | Rha-C10:1C8 | 633840 | ND | ND |
| | Rha-C10:1C10 | 1555974 | ND | ND |
| | Rha-C10C8 | 174208176 | 865146 | 0.497 |
| | Rha-C10C9 | 701084 | ND | ND |
| | Rha-C10C10 | 313724128 | ND | ND |
| | Rha-C10C10:1 | 372849 | ND | ND |
| | Rha-C10C11 | 201062 | ND | ND |
| | Rha-C10C12 | 15347609 | 463631 | 3.021 |
| | Rha-C10C12:1 | 844589 | ND | ND |
| | Rha-C10C14 | 816377 | 68528 | 8.394 |
| | Rha-C10C14:1 | 3160281 | ND | ND |
| | Rha-C10C16 | 27411 | ND | ND |
| | Rha-C11C10 | 366337 | ND | ND |
| | Rha-C12:1C8 | 2439149 | ND | ND |
| | Rha-C12:1C10 | 25018042 | 865093 | 3.458 |
| | Rha-C12:1C12 | 1291091 | ND | ND |
| | Rha-C12:1C12:1 | 325098 | ND | ND |
| | Rha-C12C8 | 2900004 | ND | ND |
| | Rha-C12C10 | 30280874 | ND | ND |
| | Rha-C12C12 | 832122 | 201085 | 24.165 |
| | Rha-C12C12:1 | 57431 | ND | ND |
| | Rha-C12C14:1 | 99859 | ND | ND |
| | Rha-C14:1C10 | 2865155 | ND | ND |
| Di-rhamno- | RhaRha-C8C8 | 151578208 | 1418875 | 0.936 |
| di-lipid | RhaRha-C8C10 | 963405120 | 82897920 | 8.605 |
| | RhaRha-C8C12 | 40224272 | 6429435 | 15.984 |
| | RhaRha-C8C12:1 | 278139456 | 6011257 | 2.161 |
| | RhaRha- | 207076368 | 2935456 | 1.418 |
| | C10:1C10 | | | |
| | RhaRha- | 1107927 | ND | ND |
| | C10:1C12:1 | | | |
| | RhaRha-C10C8 | 1004911808 | 94418816 | 9.396 |
| | RhaRha-C10C10 | 3263549696 | 788014272 | 24.146 |
| | RhaRha- | 166241424 | 2550719 | 1.534 |
| | C10C10:1 | | | |
| | RhaRha-C10C12 | 951596352 | 124517272 | 13.085 |
| | RhaRha- | 1158122496 | 104077520 | 8.987 |
| | C10C12:1 | | | |
| | RhaRha-C10C14 | 124723464 | 7578224 | 6.076 |
| | RhaRha- | 361702752 | 22878010 | 6.325 |
| | C10C14:1 | | | |
| | RhaRha-C12:1C8 | 217275888 | 3790275 | 1.744 |
| | RhaRha- | 1297265280 | 89493808 | 6.899 |
| | C12:1C10 | | | |
| | RhaRha- | 143677024 | 3808457 | 2.651 |
| | C12:1C12 | | | |
| | RhaRha- | 26095004 | 1152989 | 4.418 |
| | C12:1C12:1 | | | |
| | RhaRha-C12C8 | ND | 6449920 | New |
| | | | | Molecule |
| | RhaRha-C12C10 | 1211309824 | 150630688 | 12.435 |
| | RhaRha-C12C12 | 132062784 | 6477482 | 4.905 |
| | RhaRha- | 155098160 | 3873338 | 2.497 |
| | C12C12:1 | | | |
| | RhaRha-C12C14 | 7439984 | 346153 | 4.653 |
| | RhaRha- | 351906432 | 17409038 | 4.947 |
| | C14:1C10 | | | |
| | RhaRhaC14C10 | 136522384 | 6874241 | 5.035 |

Tables 1.3a and Table 1.3b show the weight percentage of some of the species that were found in the mass spectroscopy from Table 1.2 of the hydrolyzed inventive material from Example A Scheme 1. The data in Table 1.3a was determined by using UPLC-CAD (ultra high-performance liquid chromatography-charged aerosol detector) on a sample from Example A, Scheme 1, with the table showing retention time from 2 to 12 minutes. The data shows that about 78.2% by weight of the inventive composition is rhamnomonolipids. As there are some components in the composition that are not rhamnolipids and shown in the 2 to 12 minutes retention time, the weight % of all rhamnolipids that are monolipids is likely even higher. The retention time readings from 2 to 12 minutes captures all rhamnolipids in the composition, though there may be additional materials, such as salts and unhydrolyzed starting components.

Similar analysis was performed on the starting material of Rheance One from Evonik, and Table 1.3b shows a comparison of the weight percent of rhamnomonolipids and rhamnodilipids in the starting Rheance One material and the inventive composition from Example A Scheme 1. As can be seen, the inventive composition comprises a much higher amount of rhamnomonolipid material.

TABLE 1.3a
Peak List
Retention Time (RT) from 2 to 12 minutes

| RT | Area | Wt. % | Analyte | |
|---|---|---|---|---|
| 2.89 | 0.116 | 0.28 | | |
| 3.04 | 4.266 | 10.26 | RhaRhaC8 | |
| 3.18 | 0.019 | 0.05 | | |
| 3.57 | 0.02 | 0.05 | | |
| 4.09 | 27.021 | 64.97 | RhaRhaC10 | |
| 4.18 | 0.464 | 1.12 | RhaC10 | |
| 4.45 | 0.755 | 1.82 | C10 | |
| 4.56 | 0.019 | 0.05 | | |
| 4.70 | 0.073 | 0.18 | | |
| 5.02 | 0.476 | 1.14 | RhaRhaC12 | |
| 5.21 | 0.258 | 0.62 | RhaC12 | |
| 5.45 | 0.027 | 0.06 | | |
| 5.50 | 0.023 | 0.06 | RhaRhaC14:1 | |
| 5.66 | 0.261 | 0.63 | C12 | |
| 6.18 | 0.046 | 0.11 | C14:1 | RhaRhaC14 |
| 6.47 | 0.263 | 0.6 | RhaRhaC10C8 | RhaRhaC8C10 |
| 6.91 | 0.017 | 0.04 | | |
| 7.26 | 1.864 | 4.48 | RhaRhaC10C10 | |
| 7.75 | 0.082 | 0.20 | RhaRhaC12:1C10 | RhaRhaC10C12:1 |
| 7.87 | 0.034 | 0.08 | C10C10 | |
| 8.14 | 0.155 | 0.37 | RhaRhaC12C10 | RhaRhaC10C12 |
| 8.30 | 0.612 | 1.47 | | |
| 9.49 | 4.181 | 10.05 | RhaC12C12 | |
| 9.91 | 0.19 | 0.46 | C12C12 | |
| 10.66 | 0.316 | 0.76 | RhaC12C14 | RhaC14C12 |
| 11.96 | 0.031 | 0.07 | | |

TABLE 1.3b

| | Pre-hydrolysis (wt. %) | Post-hydrolysis inventive material of |
|---|---|---|
| | (Rheance One): | Example A Scheme 1 (wt. %): |
| Rhamnomonolipids | 3.6 | 78.2 |
| Rhamnodilipids | 94 | 16.5 |

### For UPLC-CAD:

10 Chromatography consisted of an ACQUITY BEH C18 Vanguard precolumn (130 Å, 1.7 μm, 2.1 mm×5 mm, Waters, Milford, MA, USA) followed by an ACQUITY UPLC BEH C18 Column (130 Å, 1.7 μm, 2.1 mm×100 mm, Waters, Milford, MA, USA). The LC gradient was carried out on Vanquish UPLC-CAD system (Thermo Fisher Scientific, Waltham, MA, USA) from 5 to 95% B (Acetonitrile) over 10.5 min, for a total run time of 15 min with inverse gradient. Data in Table 1.3a represents results from the retention time of 2 to 12 minutes. Mobile Phase A was 4 mM Ammonium Acetate in Water, and the flow rate was set to 0.4 mL/min. The column temperature was kept at 45° C. in the column compartment. All data processing was performed using the Xcalibur Software.

The peak identity for rhamnolipids is confirmed by LC-MS/MS (liquid chromatography (LC) tandem mass spectrometry (MS).

Eluted rhamnolipids from the UPLC column were analyzed by tandem mass spectrometry on a Triple Quad 6500+ system (AB Sciex, Framingham, MA, USA). The instrument was operated in multiple reaction monitoring (MRM) and negative electrospray ionization (ESI) mode.

All data processing was performed using the Skyline Software, Version 23.1.0.268 (MacCoss Lab, University of Washington, Seattle, WA, USA). The transition results were exported to CSV file for further analysis. For relative quantitation purpose, carbon-13 stable isotope labeled Rhamnolipids was prepared by fermentation using D-glucose (U-13C6) as the sole carbon source. The list of rhamnolipids and corresponding transitions is included in Table 1.2.

### Antimicrobial Effect

The inventive compositions herein may have a beneficial antimicrobial effect. For example, Tables 1.4 and 1.5 show the antimicrobial effect of the Example A Scheme 1 inventive composition on E. coli (Escherichia coli) and S. aureus (Staphylococcus aureus), as compared to a commercial rhamnolipid.

Table 1.4b shows the Minimal Kill Concentration Assay (MKCA), which determines the minimum concentration needed to kill the microbial component. As shown in the table 1.4b, the inventive material from Example A Scheme 1 has a greater antimicrobial effect than the commercial rhamnolipid. Similarly, individual monorhamnomonolipid species and individual beta hydroxy fatty acids, which are all in the hydrolyzed composition in greater amounts than they are in the commercial rhamnolipid, exhibit greater antimicrobial effect than the commercial rhamnolipid. This supports that the hydrolyzed inventive compositions would have an increased effect against microbials.

In some embodiments, an inventive composition may have a minimal kill concentration against E. coli of less than about 20,000 ppm, less than about 19,000 ppm, less than about 18,000 ppm, less than about 17,000, less than about 16,000 ppm, from about 10,000 ppm to about 20,000 ppm, from about 12,000 ppm to about 18,000 ppm, from about 14,000 ppm to about 16,000 ppm, or from about 15,000 ppm to about 16,000 ppm, as determined by the MKC test method. In some embodiments, an inventive composition may have a minimal kill concentration against S. aureus of less than about 35,000 ppm, less than about 34,000 ppm, less than about 33,000 ppm, less than about 32,000 ppm, from about 25,000 ppm to about 35,000 ppm, from about 27,000 ppm to about 33,000 ppm, from about 29,000 ppm to about 32,000 ppm, or from about 31,000 ppm to about 32,000 ppm, as determined by the MKC test method.

Table 1.5 shows MIC (Minimum Inhibitory Concentration) data for the same materials as Table 1.4, and shows that the inventive rhamnolipid has a greater antimicrobial effect than the commercial rhamnolipid, and that the individual monorhamnomonolipid species and individual beta hydroxy fatty acids, which are all in the hydrolyzed composition in greater amounts than they are in the commercial rhamnolipid, exhibit greater antimicrobial effect than the commercial rhamnolipid.

In some embodiments, an inventive composition may have a minimum inhibitory concentration against S. aureus of less than about 100 ppm, less than about 90 ppm, less than about 80 ppm, from about 50 ppm to about 100 ppm, from about 60 ppm to about 90 ppm, or from about 70 ppm to about 80 ppm, as determined by the MIC test method.

TABLE 1.4a

| | Sample Name | SMILES |
|---|---|---|
| commercial | Rhe | |
| rhamnolipid (Rheance | | |
| One) | | |
| hydrolyzed | Rhe/NaOH | CCCCCC(OC1O[C@@H](C)[C@H](O)[C@@H](O)[C@H]1O)CC(O)═O |
| rhamnolipid | | |
| Example A Scheme 1 | | |
| monorhamnomonolipids | Rha1C8 | C[C@@H]1OC(OC(CCCCCCC)CC(O)═O)[C@H](O)[C@H](O)[C@H]1O |
| | Rha1C10 | C[C@@H]1OC(OC(CCCCCCCC)CC(O)═O)[C@H](O)[C@H](O)[C@H]1O |
| | Rha1C11 | C[C@@H]1OC(OC(CCCCCCCCC)CC(O)═O)[C@H](O)[C@H](O)[C@H]1O |
| | Rha1C12 | C[C@@H]1OC(OC(CCCCCCCCCC)CC(O)═O)[C@H](O)[C@H](O)[C@H]1O |
| | Rha1C13 | C[C@@H]1OC(OC(CCCCCCCCCCC)CC(O)═O)[C@H](O)[C@H](O)[C@H]1O |
| | Rha1C14 | CCCCCC(OC1O[C@@H](C)[C@H](O)[C@@H](O)[C@H]1O)CC(O)=O |
| beta hydroxy fatty | (±)-3- | CCCCCC(O)CC(O)═O |
| acids | Hydroxyoctanoic | |
| | acid | |
| | (±)-3- | CCCCCCCC(O)CC(O)═O |
| | Hydroxydecanoic | |
| | acid | |
| | (3R)-3- | O═C(O)CC(O)CCCCCCCCC |
| | hydroxydodecanoic | |
| | acid | |
| | DL-β- | CCCCCCCCCC(O)CC(O)═O |
| | Hydroxylauric | |
| | acid | |
| | DL-β- | CCCCCCCCCCCC(O)CC(O)═O |
| | Hydroxymyristic | |
| | acid | |
| | DL-β- | CCCCCCCCCCCCCC(O)CC(O)═O |
| | Hydroxypalmitic | |
| | acid | |

TABLE 1.4b

| | | | E. coli MKC | S. aureus |
|---|---|---|---|---|
| | Sample Name | Solvent | (ppm) | MKC (ppm) |
| Commercial Rhamnolipid | Rhe | water | >100000 | >100000 |
| (Rheance One) | | | | |
| Hydrolyzed rhamnolipid | Rhe/NaOH | water | 25000 | 25000 |
| (Example A Scheme 1) | | | | |
| Monorhamnolipids | Rha1C8 | water | >1225 | >1225 |
| | Rha1C10 | water | >1338 | >1338 |
| | Rha1C11 | water | 1394 | 1394 |
| | Rha1C12 | water | >1450 | 725 |
| | Rha1C13 | DMSO | 753 | 1506 |
| | Rha1C14 | DMSO | 781 | 781 |
| beta hydroxy fatty acids | (±)-3- | water | >641 | >641 |
| | Hydroxyoctanoic | | | |
| | acid | | | |
| | (±)-3- | water | 753 | 753 |
| | Hydroxydecanoic | | | |
| | acid | | | |
| | (3R)-3- | water | >865 | 865 |
| | hydroxydodecanoic | | | |
| | acid | | | |
| | DL-β- | water | >865 | >865 |
| | Hydroxylauric acid | | | |
| | DL-β- | DMSO | 122 | 244 |
| | Hydroxymyristic | | | |
| | acid | | | |
| | DL-β- | DMSO | 136 | >1090 |
| | Hydroxypalmitic | | | |
| | acid | | | |

TABLE 1.5

| | | | S. aureus | |
|---|---|---|---|---|
| | | MIC | MIC | MBC |
| | Sample Name | Solvent | (ppm) | (ppm) |
| commercial rhamnolipid | Rhe | Water | 10000 | >10000 |
| (Rheance One) | | | | |
| hydrolyzed rhamnolipid | Rhe/NaOH | DMSO | 78 | 78 |
| (Example A Scheme 1) | | | | |
| monorhamnomonolipids | Rha1C8 | DMSO | 10000 | >10000 |
| | Rha1C10 | DMSO | >10000 | >10000 |
| | Rha1C11 | DMSO | 2500 | >10000 |
| | Rha1C12 | DMSO | 1250 | >10000 |
| | Rha1C13 | DMSO | 2500 | >10000 |
| | Rha1C14 | DMSO | 625 | >10000 |
| beta hydroxy fatty acids | (±)-3- | | | |
| | Hydroxyoctanoic acid | | | |
| | (±)-3- | DMSO | 1130 | >1130 |
| | Hydroxydecanoic acid | | | |
| | (3R)-3- | | | |
| | hydroxydodecanoic | | | |
| | acid | | | |
| | DL-β-Hydroxylauric | DMSO | 325 | 650 |
| | acid | | | |
| | DL-β- | DMSO | 365 | 731 |
| | Hydroxymyristic acid | | | |
| | DL-β- | DMSO | 102 | 204 |
| | Hydroxypalmitic acid | | | |

### Example B Synthesis of ReNuva-RL50 Hydrolysate Sodium Salt

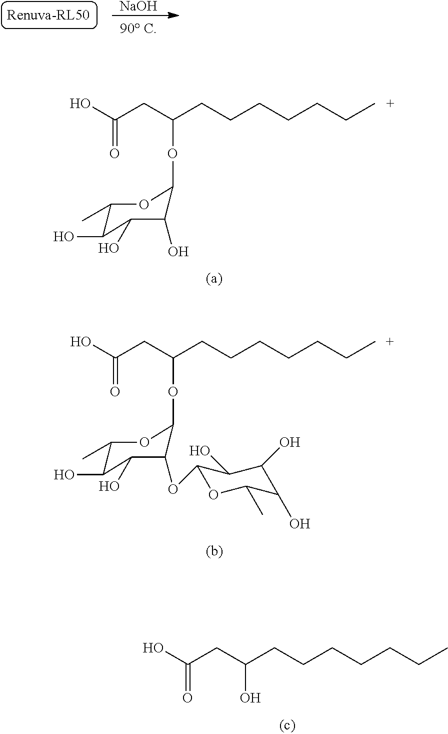

In a 2 L, 3-neck round-bottomed flask equipped with reflux condenser with N₂ inlet regulator, Caframo RZR-2000 Overhead Stirrer, and J-Kem Scientific Temperature Controller with probe was charged with a 500 grams weight solution of ReNuva RL-50, (CAS #148409-20-5, 50% weight in H₂O, 250 g, Mw˜683, 0.37 mol), followed by NaOH pellets (CAS 1310-73-2, Mw-40, 30 grams, 0.74 mol). The reaction was heated to 90° C. and stirred for 3 hours. The solution darkened over this time. The reaction was cooled to room temperature and was then lyophilized to remove water. (Virtis Model #50L Virtual XL-70, 130 mTorr, 72 hrs, 0° C.-room temperature shelf temperature). The result was a reddish, tacky solid (230 grams), which was used without further purification, predominantly as a mixture of (b), and (c), in greater than 25%

### Example C Synthesis of Carfil BIO-RL-1 Hydrolysate Sodium Salt

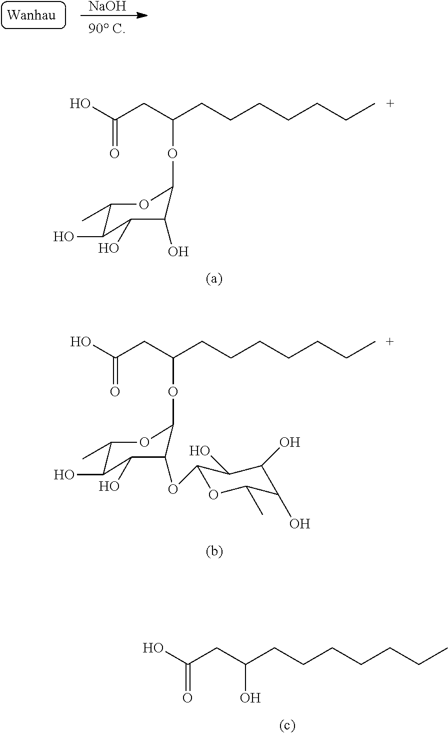

In a 2 L, 3-neck round-bottomed flask equipped with reflux condenser with N₂ inlet regulator, Caframo RZR-2000 Overhead Stirrer, and J-Kem Scientific Temperature Controller with probe was charged with a 658 grams weight solution of Carfil BIO-RLI (Wanhua, 38% weight in H₂O, 250 g, Mw˜683, 0.37 mol), followed by NaOH pellets (CAS 1310-73-2, Mw=40, 30 grams, 0.74 mol). The reaction was heated to 90° C. and stirred for 3 hours. The solution darkened over this time. The reaction was cooled to room temperature and was then lyophilized to remove water. (Virtis Model #50L Virtual XL-70, 130 mTorr, 72 hrs, 0° C.-room temperature shelf temperature). The result was a reddish, tacky solid (230 grams), which was used without further purification, predominantly as a mixture of (b), and (c), in greater than 25%.

### Example D

### Synthesis of Methyl 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decanoate (Example-(b)

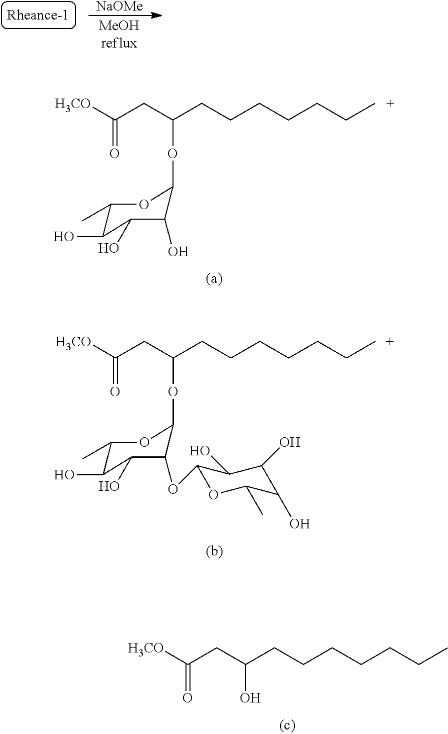

In a 250 mL round-bottomed flask equipped with a reflux condenser and stir bar was added Rheance One (Evonik, 100% wt after lyophilization, 5 g, Mw˜683, 0.0073 mol) as an off-white powder. To this was added methanol (MeOH, 75 mL), followed by solid Sodium Methoxide (NaOMe, TCI Chemicals, CAS #124-41-4, Lot #5VI5C-EA, Mw=54.02, 0.0153 mol, 0.83 grams) in a single portion. The reaction was stirred at RT for 30 minutes, then refluxed 3 hrs. The reaction was cooled to room temperature; solvent was removed via rotary evaporator, and the residue was pumped on under vacuum 24 hours at room temperature. The yield was 5.5 grams of tacky solid which was a mixture of (a), (b), and (c), predominantly enriched in (b) and (c).

### Scheme 2

Starting with Example A above and isolating the acid 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decanoic acid (that is, using (b)'s in Table 1 as a starting material), the following Rha-Rha glycolipids, Table 2 (b)'s below, are obtained by those skilled in the art (also shown in Examples E-H). These novel and inventive methods and compositions (Table 2, Structures (b) 1-11) have been shown to possess anti-bacterial activity (Table 4 and Table 5).

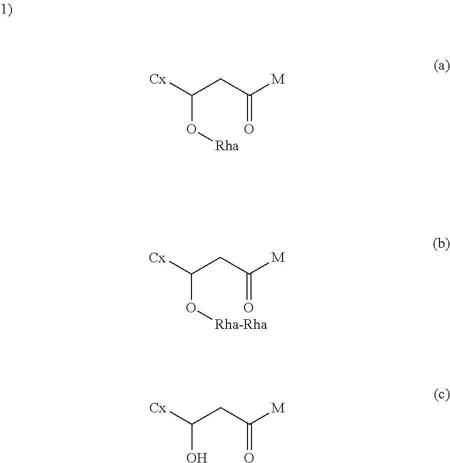

TABLE 2

| | | | Structure | |
|---|---|---|---|---|
| Entry | Reagent | Structure (b) | (c) | Name (b) |
| 1 | CH3O−Na+ | 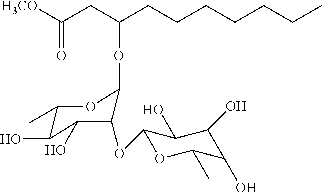 [SMILES: CCCCCCCC(CC(=O)OC)OC1OC(C)C(O)C(O)C1OC1OC(C)C(O)C(O)C1O] | | Methyl 3-(((2R,3R,4R,5R,6S)-4,5-dihydroxy- 6-methyl-3-(((2R,3S,4S,5S,6S)-3,4,5- trihydroxy-6-methyltetrahydro-2H-pyran-2- yl)oxy)tetrahydro-2H-pyran-2- yl)oxy)decanoate |
| 2 | iPrO−Na+ | 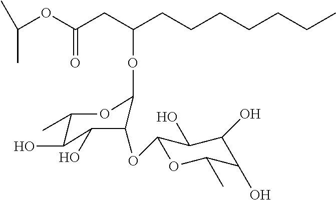 [SMILES: CCCCCCCC(CC(=O)OC(C)C)OC1OC(C)C(O)C(O)C1OC1OC(C)C(O)C(O)C1O] | | Isopropyl 3-(((2R,3R,4R,5R,6S)-4,5-dihydroxy- 6-methyl-3-(((2R,3S,4S,5S,6S)- 3,4,5-trihydroxy-6-methyltetrahydro-2H- pyran-2-yl)oxy)tetrahydro-2H-pyran-2- yl)oxy)decanoate |
| 3 | NH(CH3)2 | 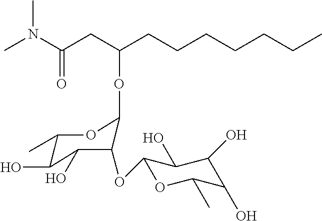 [SMILES: CCCCCCCC(CC(=O)N(C)C)OC1OC(C)C(O)C(O)C1OC1OC(C)C(O)C(O)C1O] | | 3-(((2R,3R,4R,5R,6S)-4,5-dihydroxy-6- methyl-3-(((2R,3S,4S,5S,6S)-3,4,5- trihydroxy-6-methyltetrahydro-2H-pyran-2- yl)oxy)tetrahydro-2H-pyran-2-yl)oxy)-N,N- dimethyldecanamide |
| 4 | Ethanolamine | 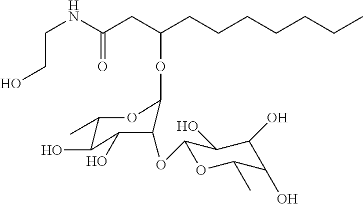 [SMILES: CCCCCCCC(CC(=O)NCCO)OC1OC(C)C(O)C(O)C1OC1OC(C)C(O)C(O)C1O] | | 3-(((2R,3R,4R,5R,6S)-4,5-dihydroxy-6- methyl-3-(((2R,3S,4S,5S,6S)-3,4,5- trihydroxy-6-methyltetrahydro-2H-pyran-2- yl)oxy)tetrahydro-2H-pyran-2-yl)oxy)-N-(2- hydroxyethyl)decanamide |
| 5 | taurine | 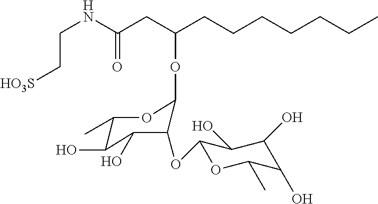 [SMILES: CCCCCCCC(CC(=O)NCCS(=O)(=O)O)OC1OC(C)C(O)C(O)C1OC1OC(C)C(O)C(O)C1O] | | 3-(((2R,3R,4R,5R,6S)-4,5-dihydroxy-6- methyl-3-(((2R,3S,4S,5S,6S)-3,4,5- trihydroxy-6-methyltetrahydro-2H-pyran-2- yl)oxy)tetrahydro-2H-pyran-2-yl)oxy)- decanamido)ethane-1-sulfonic acid |
| 6 | Me−Mg+Br | 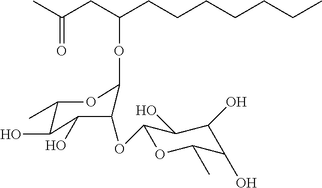 [SMILES: CCCCCCCC(CC(C)=O)OC1OC(C)C(O)C(O)C1OC1OC(C)C(O)C(O)C1O] | | 4-(((2R,3R,4R,5R,6S)-4,5-dihydroxy-6- methyl-3-(((2R,3S,4S,5S,6S)-3,4,5- trihydroxy-6-methyltetrahydro-2H-pyran-2- yl)oxy)tetrahydro-2H-pyran-2- yl)oxy)undecane-2-one |
| 7 | Bn−Mg+Cl | 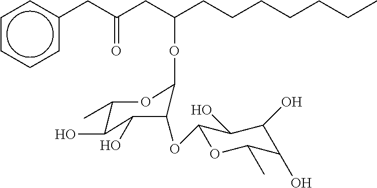 | | 4-(((2R,3R,4R,5R,6S)-4,5-dihydroxy-6- methyl-3-(((2R,3S,4S,5S,6S)-3,4,5- trihydroxy-6-methyltetrahydro-2H-pyran-2- yl)oxy)tetrahydro-2H-pyran-2-yl)oxy)-1- phenylundecan-2-one |
| 8 | NH2(CH3) | 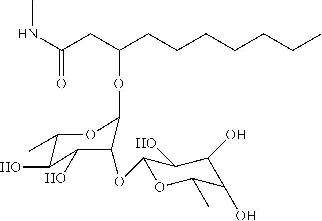 [SMILES: CCCCCCCC(CC(=O)NC)OC1OC(C)C(O)C(O)C1OC1OC(C)C(O)C(O)C1O] | | 3-(((2R,3R,4R,5R,6S)-4,5-dihydroxy-6- methyl-3-(((2R,3S,4S,5S,6S)-3,4,5- trihydroxy-6-methyltetrahydro-2H-pyran-2- yl)oxy)tetrahydro-2H-pyran-2-yl)oxy)-N- methyldecanamide |
| 9 | benzylamine | 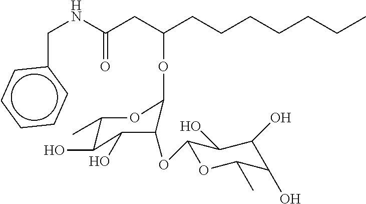 | | N-benzyl-3-(((2R,3R,4R,5R,6S)-4,5- dihydroxy-6-methyl-3-(((2R,3S,4S,5S,6S)- 3,4,5-trihydroxy-6-methyltetrahydro-2H- pyran-2-yl)oxy)tetrahydro-2H-pyran-2- yl)oxy)decanamide |
| 10 | Glycine methyl ester | 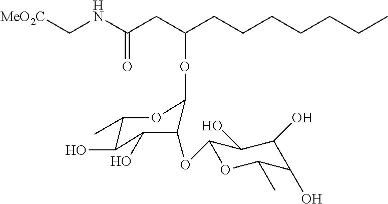 [SMILES: CCCCCCCC(CC(=O)NCC(=O)OC)OC1OC(C)C(O)C(O)C1OC1OC(C)C(O)C(O)C1O] | | methyl (3-((2R,3R,4R,5R,6S)-4,5-dihydroxy- 6-methyl-3-(((2R,3S,4S,5S,6S)-3,4,5- trihydroxy-6-methyltetrahydro-2H-pyran-2- yl)oxy)tetrahydro-2H-pyran-2- yl)oxy)decanoyl)glycinate |
| 11 | iPr−Mg+Br | 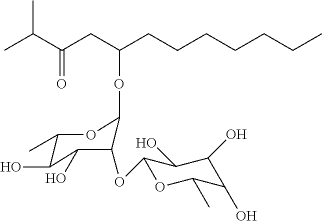 [SMILES: CCCCCCCC(CC(=O)C(C)C)OC1OC(C)C(O)C(O)C1OC1OC(C)C(O)C(O)C1O] | | 4-(((2R,3R,4R,5R,6S)-4,5-dihydroxy-6- methyl-3-(((2R,3S,4S,5S,6S)-3,4,5- trihydroxy-6-methyltetrahydro-2H-pyran-2- yl)oxy)tetrahydro-2H-pyran-2-yl)oxy)-2- methyldodecan-3-one |

### Example E

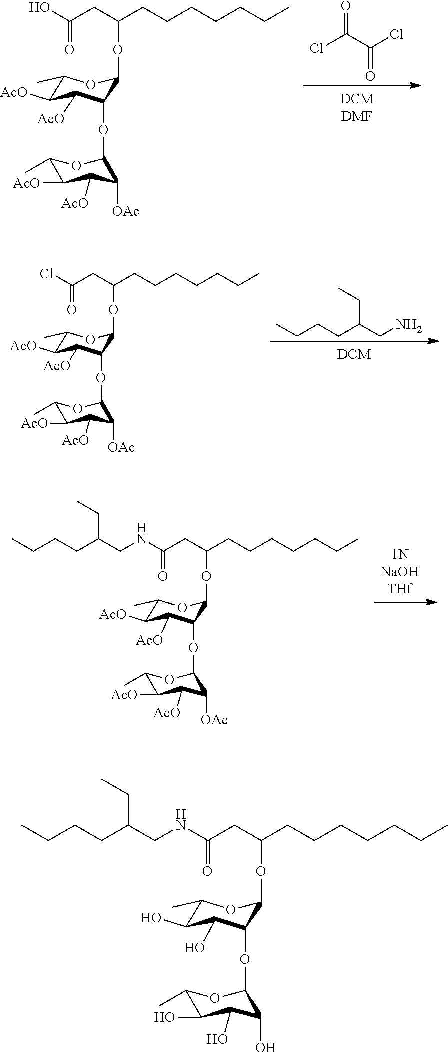

### Production of (2S,3R,4R,5S,6S)-2-(((2R,3R,4R,5S,6S)-4,5-diacetoxy-2-((1-((2-ethylhexyl)amino)-1-oxodecan-3-yl)oxy)-6-methyltetrahydro-2H-pyran-3-yl)oxy)-6-methyltetrahydro-2H-pyran-3,4,5-triyl triacetate

To a 25-mL, single neck, round bottom reaction flask was added 290 milligrams (0.4198 millimoles) of 3-(((2R,3R,4R,5S,6S)-4,5-diacetoxy-6-methyl-3-(((2S,3R,4R,5S,6S)-3,4,5-triacetoxy-6-methyltetrahydro-2H-pyran-2-yl)oxy)tetrahydro-2H-pyran-2-yl)oxy)decanoic acid, 3 mL anhydrous dichloromethane (Sigma-Aldrich Product #270997), and a magnetic stir bar at room temperature to produce a light yellow solution. The reaction was placed under nitrogen atmosphere through a nitrogen line at the top leading to a gas bubbler. To the reaction flask was added 0.11 mL oxalyl chloride (Sigma-Aldrich Product #O8801) and 2 drops of N,N-dimethylformamide (Sigma-Aldrich Product #227056). The reaction was stirred at room temperature under nitrogen atmosphere for 1.5 hours. Evaporation was taken to completeness using a rotary evaporator with the water bath set at 40° C. to yield an orange solid. The product, (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate was used in the next step without further purification. To a 25-mL, single neck, round bottom reaction flask containing crude (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate was added a magnetic stir bar, 3 mL dichloromethane (Sigma-Aldrich Product #270997), and 0.34 mL of 2-ethylhexyl amine (Sigma-Aldrich Product #E29508) slowly at room temperature under nitrogen atmosphere through a nitrogen line at the top leading to a gas bubbler to produce an orange solution. The reaction was stirred for 1.5 hours. Evaporation was taken to completeness using a rotary evaporator with the water bath set at 40° C. to produce an orange oil. The crude residue was purified via column chromatography (Silica gel 60 0.063-0.200 mm for column chromatography, Sigma-Aldrich Product #1.07734), eluting with 25% ethyl acetate (EMD Millipore Product #EX025P-1)—75% ethyl acetate in hexanes (EMD Millipore Product #HX0299-6) to yield a colorless residue (63 milligrams, 11% yield over two steps).

Use of UPLC-CAD, (also referred to as LC/MS), shows that the material derived is the expected chemical structure: m/z (molecular weight calculated based on the molecular weight of the atoms) calc. for C₄₀H₆₈NO₁₅⁺ [M+1]⁺: 802.46 m/z, found 802.5 m/z.

The next step is production of 3-(((2R,3R,4R,5R,6S)-4,5-dihydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)-N-(2-ethylhexyl) decanamide:

To a 20 mL scintillation vial equipped with a magnetic stir bar was added 63 milligrams (0.0786 millimoles) of (2S,3R,4R,5S,6S)-2-(((2R,3R,4R,5S,6S)-4,5-diacetoxy-2-((1-((2-ethylhexyl)amino)-1-oxodecan-3-yl)oxy)-6-methyltetrahydro-2H-pyran-3-yl)oxy)-6-methyltetrahydro-2H-pyran-3,4,5-triyl triacetate and 1.3 mL of tetrahydrofuran (EMD Millipore Product #TX0279P-1) to yield a light amber solution. While stirring, 0.4 mL of 1 N sodium hydroxide (JT Baker Product #5635) was added dropwise to the scintillation vial to yield a biphasic solution. The reaction was lightly capped and heated to 50° C. with continued stirring. After 2 hours, added another 0.2 mL of 1 N sodium hydroxide (JT Baker Product #5635) and increased the temperature to 60° C. with continued stirring. After 1 hour, another 0.2 mL of 1 N sodium hydroxide (JT Baker Product #5635) was added with continued stirring for an additional 3 hours. The reaction was cooled to room temperature and poured into a separatory funnel, facilitating transfer with ethyl acetate (EMD Millipore Product #EX025P-1). The organic layer was separated, and the aqueous layer was re-extracted with ethyl acetate (EMD Millipore Product #EX025P-1), dried with magnesium sulfate (Sigma-Aldrich Product #208094), and filtered into a single-neck round bottom flask. Evaporation was taken to completeness using a rotary evaporator with the water bath set at 40° C. The crude residue was purified via column chromatography (Silica gel 60 0.063-0.200 mm for column chromatography, Sigma-Aldrich Product #1.07734) eluting with 10%-20% methanol (EMD Millipore Product #MXD475P-1) in dichloromethane (EMD Millipore Product #DX0835P-4) with 0.1% glacial acetic acid (VWR Product #AX0073-6) to yield a colorless oil (20.8 milligrams, 45% yield).

LC/MS: m/z calc. for C₃₀H₅₈NO₁₀⁺ [M+H]⁺: 592.41 m/z, found 592.4 m/z. (Not in Table 2 (b), but made similarly and is similar to compositions in Table 2 (b).

### Example F

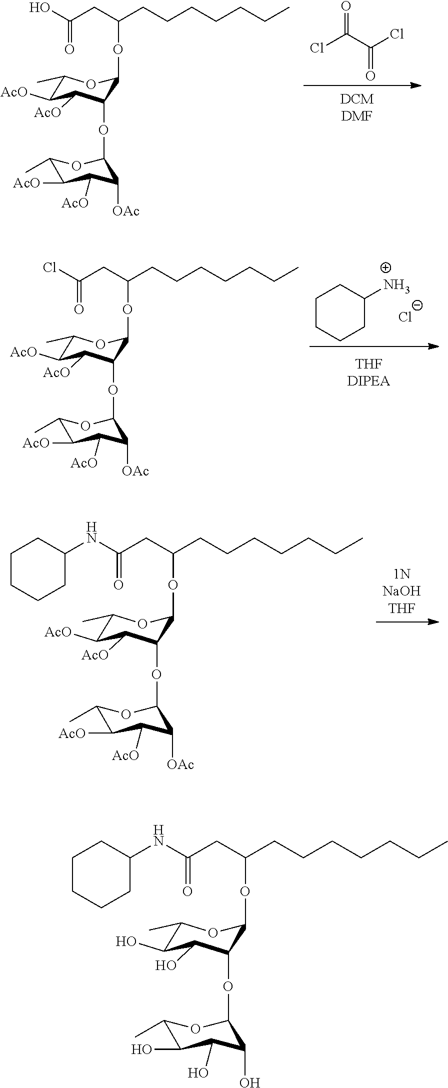

### Production of (2S,3R,4R,5S,6S)-2-(((2R,3R,4R,5S,6S)-4,5-diacetoxy-2-((1-(cyclohexylamino)-1-oxodecan-3-yl)oxy)-6-methyltetrahydro-2H-pyran-3-yl)oxy)-6-methyltetrahydro-2H-pyran-3,4,5-triyl triacetate

To a 25-mL, single neck, round bottom reaction flask was added 1.1117 grams (1.609 millimoles) of 3-(((2R,3R,4R,5S,6S)-4,5-diacetoxy-6-methyl-3-(((2S,3R,4R,5S,6S)-3,4,5-triacetoxy-6-methyltetrahydro-2H-pyran-2-yl)oxy)tetrahydro-2H-pyran-2-yl)oxy)decanoic acid, 11.50 mL anhydrous dichloromethane (Sigma-Aldrich Product #270997), and a magnetic stir bar at room temperature to produce a light yellow solution. The reaction was placed under nitrogen atmosphere through a nitrogen line at the top leading to a gas bubbler. To the reaction flask was added 0.41 mL oxalyl chloride (Sigma-Aldrich Product #O8801) and 3 drops of N,N-dimethylformamide (Sigma-Aldrich Product #227056). The reaction was stirred at room temperature under nitrogen atmosphere for 1 hour to yield a dark orange solution. Evaporation was taken to completeness using a rotary evaporator with the water bath set at 40° C. to yield an orange oil/residue. The product, (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate was used in the next step without further purification. To a 50-mL, single neck, round bottom reaction flask was added 220 milligrams cyclohexylamine hydrochloride (Ambeed Product #A120346), 2 mL of tetrahydrofuran (Sigma-Aldrich Product #186562) and 0.64 mL of N,N-Diisopropylethylamine (Sigma-Aldrich Product #387649) at room temperature under nitrogen atmosphere through a nitrogen line at the top leading to a gas bubbler for 15 min. To a separate 25-mL, single neck, round bottom reaction flask was added crude (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate and 4.5 mL of tetrahydrofuran (Sigma-Aldrich Product #186562) to produce an orange suspension which was then added to the round bottom flask containing cyclohexylamine over 15 minutes slowly to produce an orange suspension. The reaction was stirred for 2 hours at room temperature. The suspension was concentrated using a rotary evaporator to yield a brown oily residue. The crude residue was purified by column chromatography (Silica gel 60 0.063-0.200 mm for column chromatography, Sigma-Aldrich Product #1.07734), eluting in 10% ethyl acetate (EMD Millipore Product #EX025P-1)/hexanes (EMD Millipore Product #HX0299-6) to 50% ethyl acetate/hexanes to yield an orange solid product (311 milligrams, 25% yield over two steps). LC/MS: m/z calc. for C₃₈H₆₂NO₁₅⁺ [M+H]⁺: 772.41 m/z, found 772.5 m/z

### Production of N-cyclohexyl-3-(((2R,3R,4R,5R,6S)-4,5-dihydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)decanamide

To a 20 mL scintillation vial equipped with a magnetic stir bar was added 311 milligrams (0.4032 millimoles) of (2S,3R,4R,5S,6S)-2-(((2R,3R,4R,5S,6S)-4,5-diacetoxy-2-((1-(cyclohexylamino)-1-oxodecan-3-yl)oxy)-6-methyltetrahydro-2H-pyran-3-yl)oxy)-6-methyltetrahydro-2H-pyran-3,4,5-triyl triacetate and 6.7 mL of tetrahydrofuran (EMD Millipore Product #TX0279P-1) to yield a light amber solution. While stirring, 2.1 mL of 1 N sodium hydroxide (JT Baker Product #5635) was added dropwise to the scintillation vial to yield a biphasic orange suspension. The reaction was lightly capped and heated to 50° C. with continued stirring for 5 hours. The reaction was cooled to room temperature and poured into a separatory funnel, facilitating transfer with ethyl acetate (EMD Millipore Product #EX025P-1). The organic layer was separated, and the aqueous layer was re-extracted with ethyl acetate (EMD Millipore Product #EX025P-1), dried with magnesium sulfate (Sigma-Aldrich Product #208094), and filtered into a single-neck round bottom flask. Evaporation was taken to completeness using a rotary evaporator with the water bath set at 40° C. The crude residue was purified via column chromatography (Silica gel 60 0.063-0.200 mm for column chromatography, Sigma-Aldrich Product #1.07734) eluting with 0%-10% methanol (EMD Millipore Product #MXD475P-1) in dichloromethane (EMD Millipore Product #DX0835P-4) with 0.1% glacial acetic acid (VWR Product #AX0073-6) to yield a colorless oil (24.8 milligrams, 11% yield).

LC/MS: m/z calc. for C₂₈H₅₁NNaO₁₀⁺ [M+Na]⁺: 584.34 m/z, found 584.4 m/z (Not in Table 2, but prepared similarly to Table 2 (b) compositions and is similar to Table 2 (b) compositions).

### Example G

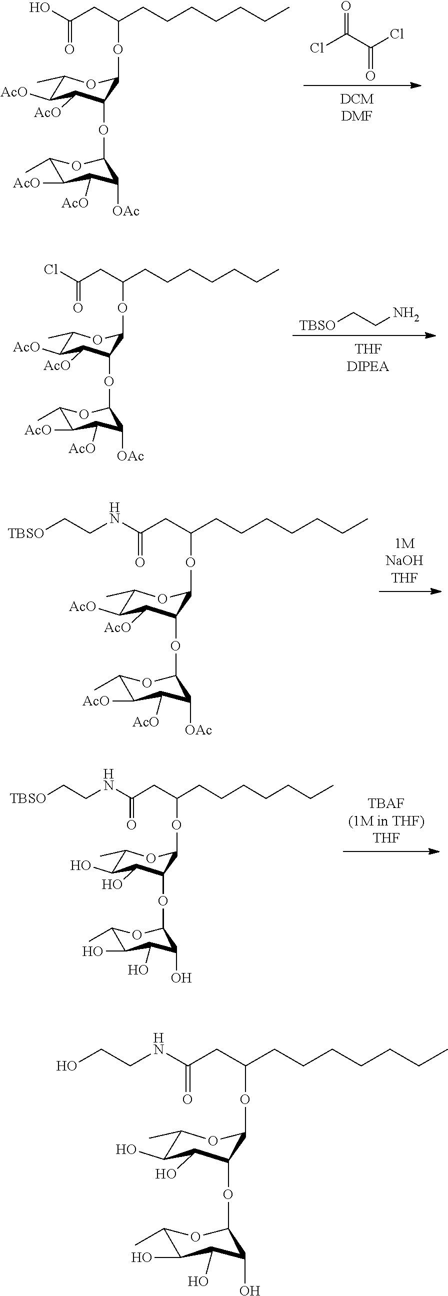

### Production of (2S,3R,4R,5S,6S)-2-(((2R,3R,4R,5S,6S)-4,5-diacetoxy-2-((1-((2-((tert-butyldimethylsilyl)oxy)ethyl)amino)-1-oxodecan-3-yl)oxy)-6-methyltetrahydro-2H-pyran-3-yl)oxy)-6-methyltetrahydro-2H-pyran-3,4,5-triyl triacetate

To a 25-mL, single neck, round bottom reaction flask was added 440 milligrams (0.6370 millimoles) of 3-(((2R,3R,4R,5S,6S)-4,5-diacetoxy-6-methyl-3-(((2S,3R,4R,5S,6S)-3,4,5-triacetoxy-6-methyltetrahydro-2H-pyran-2-yl)oxy)tetrahydro-2H-pyran-2-yl)oxy)decanoic acid, 4.60 mL anhydrous dichloromethane (Sigma-Aldrich Product #270997), and a magnetic stir bar at room temperature to produce a light yellow solution. The reaction was placed under nitrogen atmosphere through a nitrogen line at the top leading to a gas bubbler. To the reaction flask was added 0.16 mL oxalyl chloride (Sigma-Aldrich Product #O8801) and 1 drop of N,N-dimethylformamide (Sigma-Aldrich Product #227056). The reaction was stirred at room temperature under nitrogen atmosphere for 1 hour to yield a dark orange solution. Evaporation was taken to completeness using a rotary evaporator with the water bath set at 40° C. to yield an off-white residue/solid. The product, (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate was used in the next step without further purification. To a 25-mL, single neck, round bottom reaction flask was added 116 milligrams of 2-(tert-butyldimethylsilyloxy) ethanamine (Ambeed Product #A192081), 1 mL of tetrahydrofuran (Sigma-Aldrich Product #186562) and 0.13 mL of N,N- Diisopropylethylamine (Sigma-Aldrich Product #387649) at room temperature under nitrogen atmosphere through a nitrogen line at the top leading to a gas bubbler for 15 min. To a separate 25-mL, single neck, round bottom reaction flask was added crude (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate and 1.6 mL of tetrahydrofuran (Sigma-Aldrich Product #186562) to produce an orange suspension which was then added to the round bottom flask containing cyclohexylamine over 15 minutes slowly to produce an orange suspension. The reaction was stirred for 2.5 hours at room temperature. The suspension was concentrated using a rotary evaporator to yield a tan solid/residue. The crude residue was purified by column chromatography (Silica gel 60 0.063-0.200 mm for column chromatography, Sigma-Aldrich Product #1.07734), eluting in 3:1 hexanes (EMD Millipore Product #HX0299-6):ethyl acetate (EMD Millipore Product #EX025P-1) to 1:1 hexanes:ethyl acetate to yield a light colored oil product (357 milligrams, 66% yield over two steps).

LC/MS: m/z calc. for C₄₀H₇₀NO₁₆Si⁺ [M+H]⁺: 848.45 m/z, found 848.5 m/z

Production of N-(2-((tert-butyldimethylsilyl)oxy)ethyl)-3-(((2R,3R,4R,5R,6S)-4,5-dihydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)decanamide To a 20 mL scintillation vial equipped with a magnetic stir bar was added 357 milligrams (0.4210 millimoles) of (2S,3R,4R,5S,6S)-2-(((2R,3R,4R,5S,6S)-4,5-diacetoxy-2-((1-((2-((tert-butyldimethylsilyl)oxy)ethyl)amino)-1-oxodecan-3-yl)oxy)-6-methyltetrahydro-2H-pyran-3-yl)oxy)-6-methyltetrahydro-2H-pyran-3,4,5-triyl triacetate and 7.0 mL of tetrahydrofuran (EMD Millipore Product #TX0279P-1) to yield a light yellow solution. While stirring, 2.2 mL of 1 N sodium hydroxide (JT Baker Product #5635) was added dropwise to the scintillation vial to yield a biphasic solution. The reaction was lightly capped and heated to 62° C. with continued stirring for 4.5 hours. After this time, 1 mL of 1 N sodium hydroxide (JT Baker Product #5635) was added and the reaction continued to stir for 2.5 hours. The reaction was cooled to room temperature and poured into a separatory funnel, facilitating transfer with ethyl acetate (EMD Millipore Product #EX025P-1). The organic layer was separated, and the aqueous layer was re-extracted with ethyl acetate (EMD Millipore Product #EX025P-1), dried with magnesium sulfate (Sigma-Aldrich Product #208094), and filtered into a single-neck round bottom flask. Evaporation was taken to completeness using a rotary evaporator with the water bath set at 40° C. The crude residue was purified via column chromatography (Silica gel 60 0.063-0.200 mm for column chromatography, Sigma-Aldrich Product #1.07734) eluting with 0%-20% methanol (EMD Millipore Product #MXD475P-1) in dichloromethane (EMD Millipore Product #DX0835P-4) with 0.1% glacial acetic acid (VWR Product #AX0073-6) to yield a colorless oil (56 milligrams, 21% yield).

LC/MS: m/z calc. for C₃₀H₅₉NNaO₁₁Si⁺ [M+Na]⁺: 660.38 m/z, found 660.4 m/z

### Production of 3-(((2R,3R,4R,5R,6S)-4,5-dihydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)-N-(2-hydroxyethyl)decanamide

To a 20 mL scintillation vial equipped with a 14/20 septum was added 46 milligrams (0.0722 millimoles) of N-(2-((tert-butyldimethylsilyl)oxy)ethyl)-3-(((2R,3R,4R,5R,6S)-4,5-dihydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)decanamide, 1 mL tetrahydrofuran (Sigma-Aldrich Product #186562) and magnetic stir bar at room temperature under nitrogen atmosphere through a nitrogen line at the top leading to a gas bubbler. To the clear and colorless solution at room temperature was added 0.11 mL tetra-n-butylammonium fluoride (Sigma-Aldrich Product #216413) slowly to produce a light-yellow solution. The reaction was stirred for 3 hours at room temperature under nitrogen atmosphere. To the scintillation vial was added saturated ammonium chloride solution and ethyl acetate (EMD Millipore Product #EX025P-1). The organic and aqueous phases were separated, and the organic layer was re-extracted with 100 mL total ethyl acetate (EMD Millipore Product #EX025P-1). The combined organic layers were dried with magnesium sulfate (Sigma-Aldrich Product #208094) and filtered into a single neck round bottom reaction flask. The solution was concentrated with a rotary evaporator to yield a colorless residue. The crude residue was purified via column chromatography (Silica gel 60 0.063-0.200 mm for column chromatography, Sigma-Aldrich Product #1.07734), eluting with 15%-25% methanol (EMD Millipore Product #MXD475P-1) in dichloromethane (EMD Millipore Product #DX0835P-4) with 0.1% glacial acetic acid (VWR Product #AX0073-6) to yield a colorless residue (15 milligrams, 40% yield).

LC/MS: m/z calc. for C₂₄H₄₅NNaO₁₁⁺ [M+Na]⁺: 546.29 m/z, found 546.3 m/z (Table 2, (b) 4)

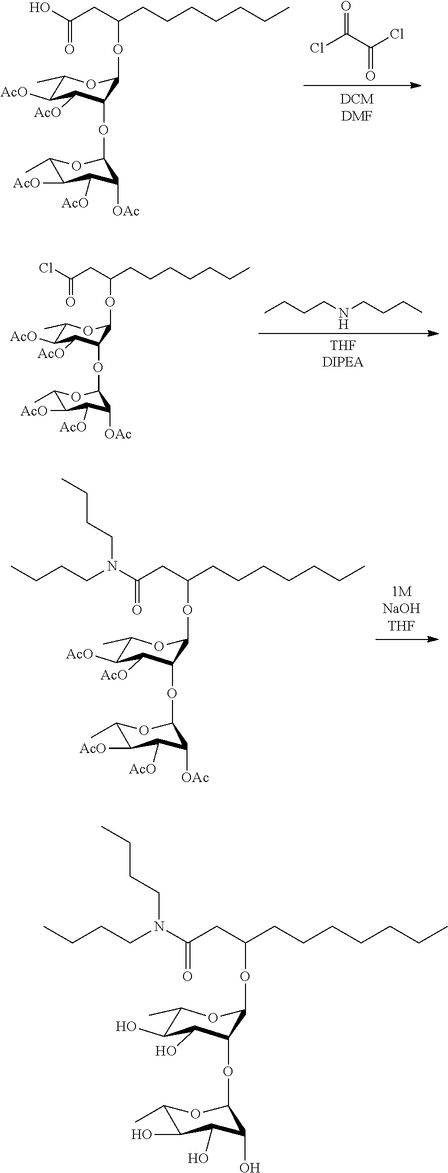

### Production of (2S,3R,4R,5S,6S)-2-(((2R,3R,4R,5S,6S)-4,5-diacetoxy-2-((1-(dibutylamino)-1-oxodecan-3-yl)oxy)-6-methyltetrahydro-2H-pyran-3-yl)oxy)-6-methyltetrahydro-2H-pyran-3,4,5-triyl triacetate

To a 25-mL, single neck, round bottom reaction flask was added 444 milligrams (0.6428 millimoles) of 3-(((2R,3R,4R,5S,6S)-4,5-diacetoxy-6-methyl-3-(((2S,3R,4R,5S,6S)-3,4,5-triacetoxy-6-methyltetrahydro-2H-pyran-2-yl)oxy)tetrahydro-2H-pyran-2-yl)oxy)decanoic acid, 4.60 mL anhydrous dichloromethane (Sigma-Aldrich Product #270997), and a magnetic stir bar at room temperature to produce a light yellow solution. The reaction was placed under nitrogen atmosphere through a nitrogen line at the top leading to a gas bubbler. To the reaction flask was added 0.16 mL oxalyl chloride (Sigma-Aldrich Product #O8801) and 1 drop of N,N-dimethylformamide (Sigma-Aldrich Product #227056). The reaction was stirred at room temperature under nitrogen atmosphere for 1 hour to yield a dark orange solution. Evaporation was taken to completeness using a rotary evaporator with the water bath set at 40° C. to yield a sticky yellow residue/solid. The product, (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate was used in the next step without further purification. To a 25-mL, single neck, round bottom reaction flask was added 0.11 mL dibutylamine (Sigma-Aldrich Product #8.03222), 1 mL of tetrahydrofuran (Sigma-Aldrich Product #186562) and 0.26 mL of N,N-Diisopropylethylamine (Sigma-Aldrich Product #387649) at room temperature under nitrogen atmosphere through a nitrogen line at the top leading to a gas bubbler for 5 min. To a separate 25-mL, single neck, round bottom reaction flask was added crude (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate and 1.6 mL of tetrahydrofuran (Sigma-Aldrich Product #186562) to produce an orange suspension which was then added to the round bottom flask containing dibutylamine over 15 minutes slowly to produce an orange suspension. The reaction was stirred for 24 hours at room temperature. The dark orange suspension was concentrated using a rotary evaporator to yield a brown oily residue. The crude residue was purified by column chromatography (Silica gel 60 0.063-0.200 mm for column chromatography, Sigma-Aldrich Product #1.07734), eluting in 10% ethyl acetate (EMD Millipore Product #EX025P-1)/hexanes (EMD Millipore Product #HX0299-6) to 20% ethyl acetate/hexanes to yield a light-yellow oily residue product (281 milligrams, 55% yield over two steps).

LC/MS: calc. for C₄₀H₆₈NO₁₅⁺ [M+H]⁺: 802.46 m/z, found 802.5 m/z

### Production of N,N-dibutyl-3-(((2R,3R,4R,5R,6S)-4,5-dihydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)decanamide

To a 20 mL scintillation vial equipped with a magnetic stir bar was added 281 milligrams (0.3504 millimoles) of ((2S,3R,4R,5S,6S)-2-(((2R,3R,4R,5S,6S)-4,5-diacetoxy-2-((1-(dibutylamino)-1-oxodecan-3-yl)oxy)-6-methyltetrahydro-2H-pyran-3-yl)oxy)-6-methyltetrahydro-2H-pyran-3,4,5-triyl triacetate and 5.8 mL of tetrahydrofuran (EMD Millipore Product #TX0279P-1) to yield a light yellow/orange solution. While stirring, 2.7 mL of 1 N sodium hydroxide (JT Baker Product #5635) was added dropwise to the scintillation vial to yield a light-yellow biphasic suspension. The reaction was lightly capped and heated to 50° C. with continued stirring for 3 hours. The reaction was cooled to room temperature and poured into a separatory funnel, facilitating transfer with ethyl acetate (EMD Millipore Product #EX025P-1). The organic layer was separated, and the aqueous layer was re-extracted with ethyl acetate (EMD Millipore Product #EX025P-1), dried with magnesium sulfate (Sigma-Aldrich Product #208094), and filtered into a single-neck round bottom flask. Evaporation was taken to completeness using a rotary evaporator with the water bath set at 40° C. The crude residue was purified via column chromatography (Silica gel 60 0.063-0.200 mm for column chromatography, Sigma-Aldrich Product #1.07734) eluting with 10%-20% methanol (EMD Millipore Product #MXD475P-1) in dichloromethane (EMD Millipore Product #DX0835P-4) with 0.1% glacial acetic acid (VWR Product #AX0073-6) to yield a colorless oil (111 milligrams, 54% yield).

LC/MS: calc. for C₃₀H₅₈NO₁₀⁺ [M+H]⁺: 592.41 m/z, found 592.4 m/z (Not in Table 2, but made similarly and is similar to Table 2 (b) compositions)

### Example I

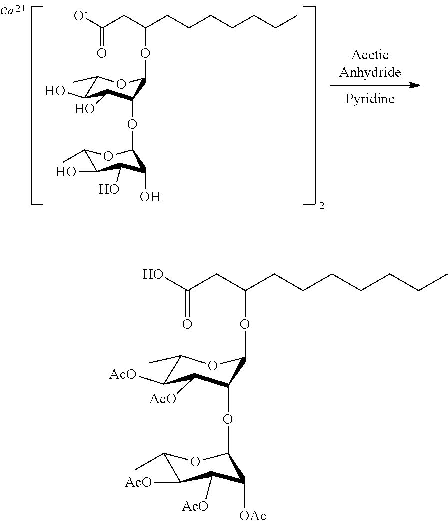

Example I shows the starting step for Examples E-H. For Examples E-H, the first step starts with the hydrolysate product from Example A, composition (b). The inventors initially protect the hydroxyl groups with tetraacetate.

### Production of 3-(((2R,3R,4R,5S,6S)-4,5-diacetoxy-6-methyl-3-(((2S,3R,4R,6R)-3,4,5-triacetoxy-6-methyltetrahydro-2H-pyran-2-yl)oxy)tetrahydro-2H-pyran-2-yl)oxy)decanoic acid

To a 250-mL, single neck, round bottom reaction flask was added 10.79 grams (10.8 millimoles) of calcium 3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)decanoate, 100 mL anhydrous pyridine (Sigma Aldrich Product #270970), and a magnetic stir bar at room temperature. The reaction was placed under nitrogen atmosphere through a nitrogen line at the top leading to a gas bubbler. To the reaction flask was added 15 mL (158.7 millimoles) acetic anhydride (Sigma Aldrich Product #539996). The reaction was stirred at room temperature under nitrogen atmosphere for 17 hours. To the reaction, 5 milligrams (0.04 millimoles) of 4-(dimethylamino)pyridine (Aldrich Product #107700), and 15 mL (158.7 millimoles) acetic anhydride (Sigma Aldrich Product #539996) was added and stirred at 50° C. under nitrogen atmosphere for 24 hours. Poured reaction into 400 mL deionized water and added 400 mL ethyl acetate (EMD Millipore Product #EX0240-6) and stirred for 30 minutes. Ethyl acetate layer was collected and extracted with 350 mL deionized water, 2×250 mL of 10% aqueous copper (II) sulfate pentahydrate (Sigma Aldrich Product #209198) solution, 2×250 mL deionized water, 250 mL brine (JT Baker Product #3624-01), 2×250 mL saturated ammonium chloride (Sigma Product #A9434), 250 mL deionized water with 50 mL brine (JT Baker Product #3624-01) added. Ethyl acetate layer was dried over sodium sulfate (Fisher Chemical Product #S429) and then filtered to remove the sodium sulfate. Evaporation of filtrate was taken to completeness using a rotary evaporator with the water bath set at 20° C. to yield a tan gummy residue. The residue was dissolved in 300 mL methylene chloride (EMD Millipore Product #DX08356-6), dried with sodium sulfate (Fisher Chemical Product #S429) for 15 hours and then filtered to remove the sodium sulfate. Evaporation of filtrate was taken to completeness using a rotary evaporator with the water bath set at 20° C. and then further dried under high vacuum (10⁻² mBar) overnight to yield a tan solid (11.7 grams, 79% yield).

LC/MS: m/z calc. for C32H49O16⁻ [M−1]⁻: 689.30 m/z, found 689.3 m/z (this material is used in Table 2 derivations)

### Example J

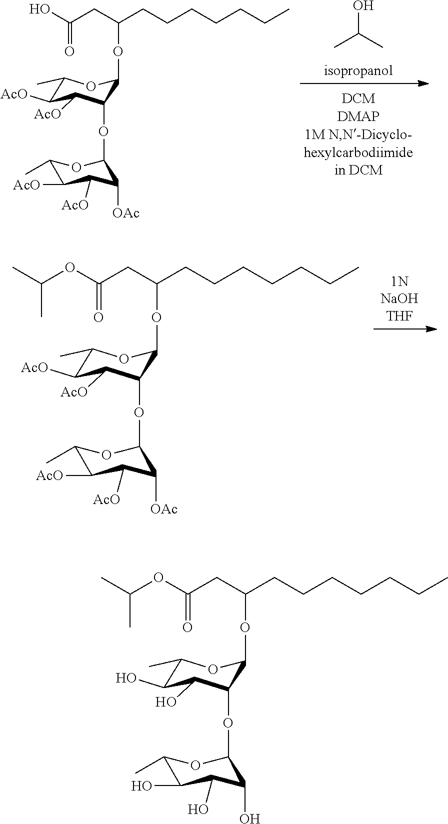

### Production of (2S,3R,4R,6R)-2-(((2R,3R,4R,5S,6S)-4,5-diacetoxy-2-((1-isopropoxy-1-oxodecan-3-yl)oxy)-6-methyltetrahydro-2H-pyran-3-yl)oxy)-6-methyltetrahydro-2H-pyran-3,4,5-triyl triacetate

To a 25-mL, single neck, round bottom reaction flask was added 536 milligrams (0.8 millimoles) of 3-(((2R,3R,4R,5S,6S)-4,5-diacetoxy-6-methyl-3-(((2S,3R,4R,5S,6S)-3,4,5-triacetoxy-6-methyltetrahydro-2H-pyran-2-yl)oxy)tetrahydro-2H-pyran-2-yl)oxy)decanoic acid, 12.7 mg (0.07 millimoles) of 4-(Dimethylamino)pyridine (Aldrich Product #107700), 0.238 mL (3.1 millimoles) 2-propanol (EMD Millipore Product #PX1835-5), 5 mL anhydrous dichloromethane (Sigma-Aldrich Product #270997), and a magnetic stir bar at room temperature to produce a light yellow solution. The reaction was placed under nitrogen atmosphere through a nitrogen line at the top leading to a gas bubbler. Cooled mixture to 0° C. in ice bath and then added 0.9 mL (0.9 millimoles) of a 1M N,N-dicyclohexylcarbodiimide/dichloromethane solution (Sigma Aldrich Product #379115) using inert atmosphere techniques. After 5 minutes of stirring at 0° C., the ice bath was removed, and the reaction was stirred for 3 hours at room temperature. Filtered the reaction through a medium pore glass filter fritted funnel to remove precipitates, rinsed the precipitates with 25 ml dichloromethane (Sigma-Aldrich Product #270997). The filtrate was extracted with 25 mL of 0.1 N hydrochloricacid (Alfa Aesar Product #35644), 25 mL of saturate sodium bicarbonate (JT Baker Product #3509-01), 25 mL brine (JT Baker Product #3624-01), dried the organic layer with sodium sulfate (Fisher Chemical Product #S429), filtered to remove the sodium sulfate. Evaporation of the filtrate was taken to completeness using a rotary evaporator with the water bath set at 35° C. and further dried under high vacuum (10-2 mBar) overnight to produce a tan oily residue. The crude residue was purified via column chromatography (Biotage 40M Silica gel column), eluting with 2% methanol (Fisher Chemical Product #A412-500) in chloroform (EMD Millipore Product #CX1054-1) with 0.1% glacial acetic acid (VWR Product #AX0073-6) to yield a tan residue (540 milligrams, 95% yield).

LC/MS: m/z calc. for C35H56NaO16⁺ [M+Na]⁺: 755.35 m/z, found 755.4 m/z

### Production of isopropyl 3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)decanoate

To a 15-mL, single neck, round bottom reaction flask was added 285 milligrams (0.4 millimoles) of (2S,3R,4R,6R)-2-(((2R,3R,4R,5S,6S)-4,5-diacetoxy-2-((1-isopropoxy-1-oxodecan-3-yl)oxy)-6-methyltetrahydro-2H-pyran-3-yl)oxy)-6-methyltetrahydro-2H-pyran-3,4,5-triyl triacetate and 6 mL of tetrahydrofuran (Fisher Chemical Product #T397-500), 1 mL (13 millimoles) 2-propanol (EMD Millipore Product #PX1835-5), and a magnetic stir bar. Cooled solution in an ice bath and then 2.0 mL of 1N sodium hydroxide (Alfa Aesar Product #35629) was added dropwise to yield a biphasic solution. Stirred for 30 minutes at 0° C. and 15 hours at room temperature. The reaction was poured into a separatory funnel and the top organic layer was collected. Evaporation of organic layer was taken to completeness using a rotary evaporator with the water bath set at 35° C. to produce a white residue. The crude residue was purified via column chromatography (Biotage 40M Silica gel column), eluting with 20% methanol (Fisher Chemical Product #A412-500) in chloroform (EMD Millipore Product #CX1054-1) with 0.1% glacial acetic acid (EMD Millipore Product #AX0073-6) to yield a crystalline solid (81 milligrams, 40% yield).

LC/MS: m/z calc. for C25H46NaO11⁺ [M+Na]⁺: 545.29 m/z, found 545.3 m/z. (Table 2 (b) 2)

### Example K

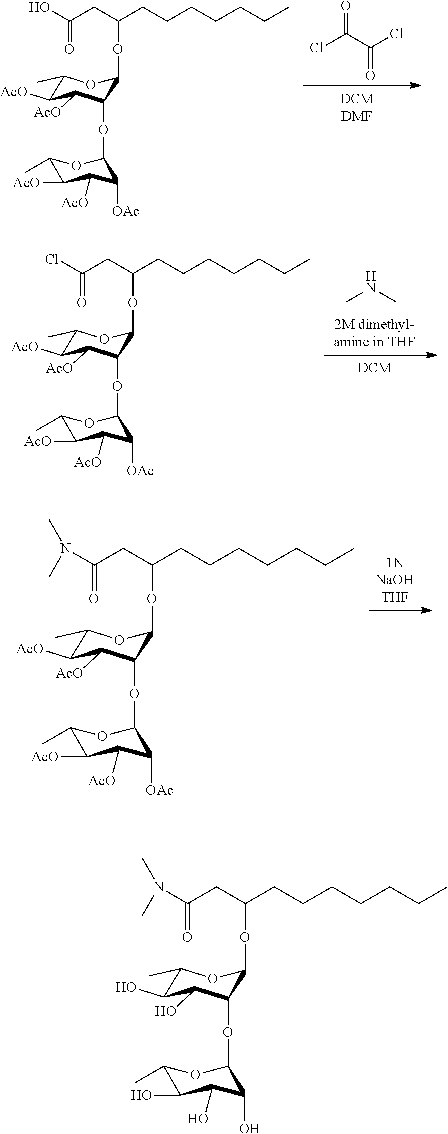

### Production of (2S,3R,4R,6R)-2-(((2R,3R,4R,5S,6S)-4,5-diacetoxy-2-((1-(dimethylamino)-1-oxodecan-3-yl)oxy)-6-methyltetrahydro-2H-pyran-3-yl)oxy)-6-methyltetrahydro-2H-pyran-3,4,5-triyl triacetate

To a 25-mL, single neck, round bottom reaction flask was added 345 milligrams (0.5 millimoles) of 3-(((2R,3R,4R,5S,6S)-4,5-diacetoxy-6-methyl-3-(((2S,3R,4R,5S,6S)-3,4,5-triacetoxy-6-methyltetrahydro-2H-pyran-2-yl)oxy)tetrahydro-2H-pyran-2-yl)oxy)decanoic acid, 5 mL anhydrous dichloromethane (Sigma-Aldrich Product #270997), and a magnetic stir bar at room temperature to produce a light yellow solution. The reaction was placed under nitrogen atmosphere through a nitrogen line at the top leading to a gas bubbler. To the reaction flask was added 0.25 mL (2.9 millimoles) oxalyl chloride (Sigma-Aldrich Product #O8801) and 2 drops of N,N-dimethylformamide (EMD Millipore Product #DX1730-6). The reaction was stirred at room temperature under nitrogen atmosphere for 1 hour. Evaporation was taken to completeness using a rotary evaporator with the water bath set at 40° C. to yield a yellow solid. The product, (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate was used in the next step without further purification.

To a 25-mL, single neck, round bottom reaction flask containing crude (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate was added a magnetic stir bar, 5 mL dichloromethane (Sigma-Aldrich Product #270997). The mixture was cooled in an ice bath and 1.25 mL (2.5 millimoles) of 2.0M dimethylamine in tetrahydrofuran (Sigma-Aldrich Product #391956) slowly added under nitrogen atmosphere through a nitrogen line at the top leading to a gas bubbler. The reaction was stirred for 1 hour at room temperature. Evaporation was taken to completeness using a rotary evaporator with the water bath set at 35° C. and further dried under high vacuum (10-2 mBar) overnight to produce an oil. The crude residue was purified via column chromatography (Biotage 40M Silica gel column), eluting with 2% methanol (Fisher Chemical Product #A412-500) in chloroform (EMD Millipore Product #CX1054-1) with 0.1% glacial acetic acid (VWR Product #AX0073-6) to yield a colorless oil (340 milligrams, 95% yield over two steps).

LC/MS: m/z calc. for C₄₀H₆₈NO₁₅⁺ [M+1]⁺: 718.36 m/z, found 718.4 m/z

### Production of 3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)-N,N-dimethyldecanamide

To a 25-mL, single neck, round bottom reaction flask was added 320 milligrams (0.4 millimoles) of (2S,3R,4R,6R)-2-(((2R,3R,4R,5S,6S)-4,5-diacetoxy-2-((1-(dimethylamino)-1-oxodecan-3-yl)oxy)-6-methyltetrahydro-2H-pyran-3-yl)oxy)-6-methyltetrahydro-2H-pyran-3,4,5-triyl triacetate and 7 mL of tetrahydrofuran (Fisher Chemical Product #T397-500) and a magnetic stir bar. While stirring, 2.27 mL of 1 N sodium hydroxide (Alfa Aesar Product #35629) was added dropwise to yield a biphasic solution. The reaction was equipped with a reflux condenser and heated at 50° C. under a nitrogen atmosphere for 3 hours. The reaction was cooled to room temperature and poured into a separatory funnel. The top organic layer was separated bottom aqueous layer. Evaporation of organic layer was taken to completeness using a rotary evaporator with the water bath set at 35° C. and further dried under high vacuum (102 mBar) overnight to produce an oil. The crude residue was purified via column chromatography (Biotage 40M Silica gel column), eluting with 20% methanol (Fisher Chemical Product #A412-500) in chloroform (EMD Millipore Product #CX1054-1) with 0.1% glacial acetic acid (EMD Millipore Product #AX0073-6) to yield a crystalline solid (123 milligrams, 54% yield).

LC/MS: m/z calc. for C24H46NO10⁺ [M+1]⁺: 508.31 m/z, found 508.3 m/z (Table 2 (b) 3)

### Example L

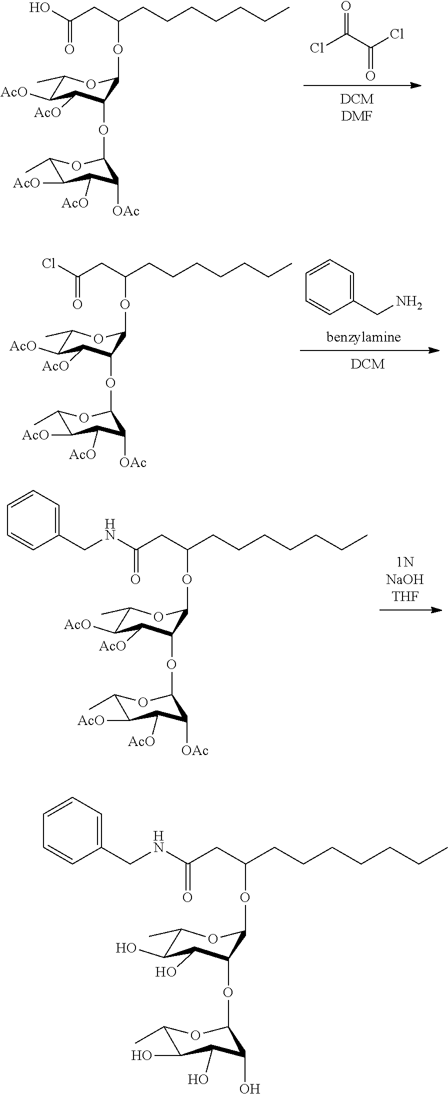

Product of (2S,3R,4R,6R)-2-(((2R,3R,4R,5S,6S)-4,5-diacetoxy-2-((1-(benzylamino)-1-oxodecan-3-yl)oxy)-6-methyltetrahydro-2H-pyran-3-yl)oxy)-6-methyltetrahydro-2H-pyran-3,4,5-triyl triacetate To a 25-mL, single neck, round bottom reaction flask was added 500 milligrams (0.7 millimoles) of 3-(((2R,3R,4R,5S,6S)-4,5-diacetoxy-6-methyl-3-(((2S,3R,4R,5S,6S)-3,4,5-triacetoxy-6-methyltetrahydro-2H-pyran-2-yl)oxy)tetrahydro-2H-pyran-2-yl)oxy)decanoic acid, 5 mL anhydrous dichloromethane (Sigma-Aldrich Product #270997), and a magnetic stir bar at room temperature to produce a light yellow solution. The reaction was placed under nitrogen atmosphere through a nitrogen line at the top leading to a gas bubbler. To the reaction flask was added 0.2 mL (2.3 millimoles) oxalyl chloride (Sigma-Aldrich Product #O8801) and 2 drops of N,N-dimethylformamide (EMD Millipore Product #DX1730-6). The reaction was stirred at room temperature under nitrogen atmosphere for 1 hour. Evaporation was taken to completeness using a rotary evaporator with the water bath set at 40° C. to yield a yellow solid. The product, (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate was used in the next step without further purification:

To a 25-mL, single neck, round bottom reaction flask containing crude (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate was added a magnetic stir bar, 5 mL dichloromethane (Sigma-Aldrich Product #270997). The mixture was cooled in an ice bath and 429 milligrams (4 millimoles) of benzylamine (Sigma Aldrich Product #185701) dissolved in 1 mL of anhydrous dichloromethane slowly added under nitrogen atmosphere through a nitrogen line at the top leading to a gas bubbler. An additional mL anhydrous methylene chloride rinse was added to the reaction vessel. The reaction was stirred for 1 hour at room temperature. Reaction was filtered to remove white precipitate, rinsed with 20 mL dichloromethane (EMD Millipore Product #DX08356-6). Evaporation of the filtrate was taken to completeness using a rotary evaporator with the water bath set at 35° C. and further dried under high vacuum (10-2 mBar) overnight to produce a tan oily semi-crystalline residue. The crude residue was purified via column chromatography (Biotage 40S Silica gel column), eluting with 2% methanol (Fisher Chemical Product #A412-500) in chloroform (EMD Millipore Product #CX1054-1) with 0.1% glacial acetic acid (VWR Product #AX0073-6) to yield a light yellow solid (500 milligrams, 88% yield over two steps).

LC/MS: m/z calc. for C39H58NO15⁺ [M+1]⁺: 780.38 m/z, found 780.4 m/z

Production of N-benzyl-3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)decanamide To a 25-mL, single neck, round bottom reaction flask was added 460 milligrams (0.6 millimoles) of (2S,3R,4R,6R)-2-(((2R,3R,4R,5S,6S)-4,5-diacetoxy-2-((1-(benzylamino)-1-oxodecan-3-yl)oxy)-6-methyltetrahydro-2H-pyran-3-yl)oxy)-6-methyltetrahydro-2H-pyran-3,4,5-triyl triacetate and 14 mL of tetrahydrofuran (Fisher Chemical Product #T397-500) and a magnetic stir bar. While stirring, 4.3 mL (4.3 millimoles) of 1 N sodium hydroxide (Alfa Aesar Product #35629) was added dropwise to yield a biphasic solution. The reaction was equipped with a reflux condenser and heated at 50° C. under a nitrogen atmosphere for 3.5 hours. The reaction was cooled to room temperature and poured into a separatory funnel. The top organic layer was separated bottom aqueous layer. Evaporation of organic layer was taken to completeness using a rotary evaporator with the water bath set at 35° C. and further dried under high vacuum (10⁻² mBar) overnight to produce a yellow residue. The crude residue was purified via column chromatography (Biotage 40S Silica gel column), eluting with 20% methanol (Fisher Chemical Product #A412-500) in chloroform (EMD Millipore Product #CX1054-1) with 0.1% glacial acetic acid (EMD Millipore Product #AX0073-6) to yield a crystalline solid (150 milligrams, 45% yield). 30

LC/MS: m/z calc. for C29H47NNaO10⁺ [M+Na]⁺: 592.31 m/z, found 592.3 m/z. (Table 2 (b) 9)

### Example M

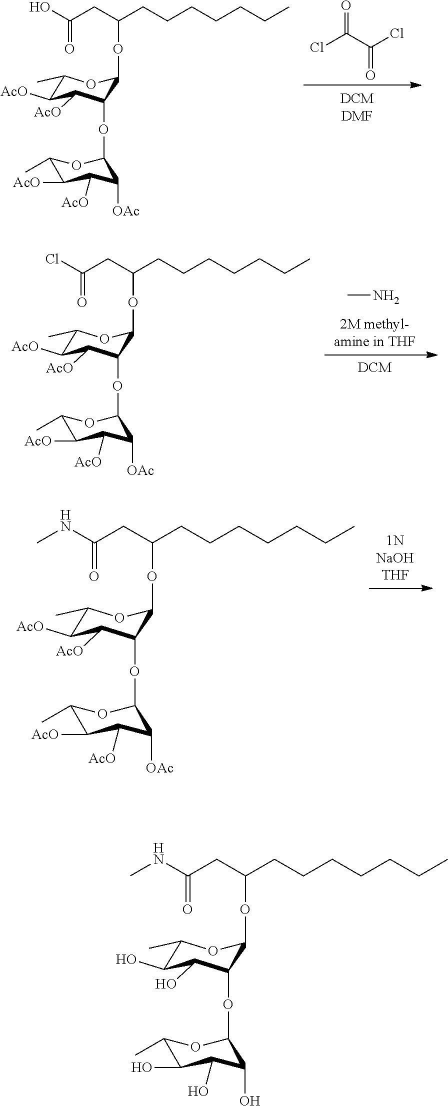

### Production of (2S,3R,4R,6R)-2-(((2R,3R,4R,5S,6S)-4,5-diacetoxy-6-methyl-2-((1-(methylamino)-1-oxodecan-3-yl)oxy)tetrahydro-2H-pyran-3-yl)oxy)-6-methyltetrahydro-2H-pyran-3,4,5-triyl triacetate

To a 25-mL, single neck, round bottom reaction flask was added 633 milligrams (0.9 millimoles) of 3-(((2R,3R,4R,5S,6S)-4,5-diacetoxy-6-methyl-3-(((2S,3R,4R,5S,6S)-3,4,5-triacetoxy-6-methyltetrahydro-2H-pyran-2-yl)oxy)tetrahydro-2H-pyran-2-yl)oxy)decanoic acid, 7 mL anhydrous dichloromethane (Sigma-Aldrich Product #270997), and a magnetic stir bar at room temperature to produce a light yellow solution. The reaction was placed under nitrogen atmosphere through a nitrogen line at the top leading to a gas bubbler. To the reaction flask was added 0.45 mL (5.3 millimoles) oxalyl chloride (Sigma-Aldrich Product #O8801) and 2 drops of N,N-dimethylformamide (EMD Millipore Product #DX1730-6). The reaction was stirred at room temperature under nitrogen atmosphere for 1.5 hours. Evaporation was taken to completeness using a rotary evaporator with the water bath set at 40° C. to yield a yellow solid. The product, (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate was used in the next step without further purification.

To a 25-mL, single neck, round bottom reaction flask containing crude (2S,3R,4R,5S,6S)-2-(((2R,3R,4R,5S,6S)-4,5-diacetoxy-2-((1-chloro-1-oxodecan-3-yl)oxy)-6-methyltetrahydro-2H-pyran-3-yl)oxy)-6-methyltetrahydro-2H-pyran-3,4,5-triyl triacetate was added a magnetic stir bar, 7 mL dichloromethane (Sigma-Aldrich Product #270997). The mixture was cooled in an ice bath and 2.8 mL (5.6 millimoles) of 2.0M methylamine in tetrahydrofuran (Sigma Aldrich Product #395056) slowly added under nitrogen atmosphere through a nitrogen line at the top leading to a gas bubbler. The reaction was stirred for 1 hour at room temperature. Filtered off the precipitate from reaction through filter paper and rinsed with dichloromethane (Sigma-Aldrich Product #270997). Evaporation was taken to completeness using a rotary evaporator with the water bath set at 35° C. and further dried under high vacuum (10-2 mBar) overnight to produce a light brown solid residue. The crude residue was purified via column chromatography (Biotage 40M Silica gel column), eluting with 1% methanol (Fisher Chemical Product #A412-500) in chloroform (EMD Millipore Product #CX1054-1) with 0.1% glacial acetic acid (VWR Product #AX0073-6) to yield an off white solid (560 milligrams, 87% yield over two steps).

LC/MS: m/z calc. for C33H54NO15⁺ [M+1]⁺: 704.35 m/z, found 704.3 m/z

### Production of 3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)-N-methyldecanamide

To a 25-mL, single neck, round bottom reaction flask was added 543 milligrams (0.8 millimoles) of (2S,3R,4R,6R)-2-(((2R,3R,4R,5S,6S)-4,5-diacetoxy-6-methyl-2-((1-(methylamino)-1-oxodecan-3-yl)oxy)tetrahydro-2H-pyran-3-yl)oxy)-6-methyltetrahydro-2H-pyran-3,4,5-triyl triacetate and 14 mL of tetrahydrofuran (Fisher Chemical Product #T397-500) and a magnetic stir bar. While stirring, 5.48 mL (5.5 millimoles) of 1 N sodium hydroxide (Alfa Aesar Product #35629) was added dropwise to yield a biphasic solution. The reaction was equipped with a reflux condenser and heated at 50° C. under a nitrogen atmosphere for 3 hours. The reaction was cooled to room temperature and poured into a separatory funnel. The top organic layer was separated bottom aqueous layer. Evaporation of organic layer was taken to completeness using a rotary evaporator with the water bath set at 35° C. and further dried under high vacuum (10⁻² mBar) overnight to produce a yellow residue. The crude residue was purified via column chromatography (Biotage 40M Silica gel column), eluting with 20% methanol (Fisher Chemical Product #A412-500) in chloroform (EMD Millipore Product #CX1054-1) with 0.1% glacial acetic acid (EMD Millipore Product #AX0073-6) to yield a white solid (160 milligrams, 42% yield).

LC/MS: m/z calc. for C23H43NNaO10⁺ [M+Na]⁺: 516.28 m/z, found 516.2 m/z. (Table 2 (b) 8)

### Example N

### Synthesis of methyl 3-(((2R,3R,4R,5R,6S)-3,4,5-trihydroxy-6-methyltetrahydro-2H-pyran-2-yl)oxy)decanoate

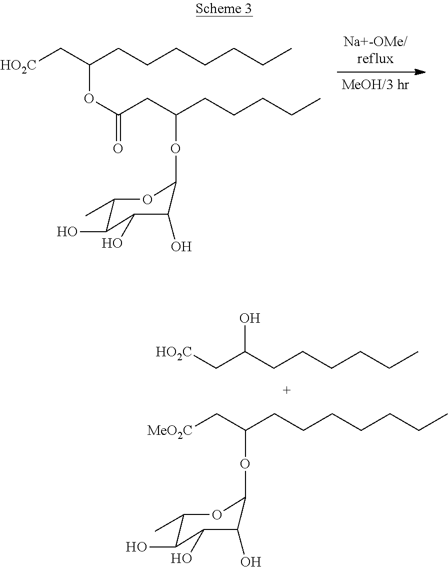

Using commercially available rhamnodilipids, inventors created novel compositions that are monorhamnomonolipids.

In a 250 mL round-bottomed flask equipped with a reflux condenser and stir bar was added Rha-(rac)-C10C10 (GlycoSurf, Salt Lake City, Utah, USA, Mw=504, 3.8 g, 0.076 mol). To this was added MeOH (75 mL), followed by solid Sodium Methoxide (TCI Chemicals, CAS #124-41-4, Lot #5VI5C-EA, Mw=54.02, 0.0153 mol, 0.83 grams) in a single portion. The reaction was stirred at RT for 30 minutes, then refluxed 3 hrs. The reaction was cooled to room temperature; solvent was removed via rotary evaporator, and the residue was pumped on under vacuum 24 hours at room temperature. The yield was 5.5 grams of tacky solid, (shown above).

Then using the Example N above and by modifying nucleophiles and solvents accordingly, the glycolipids in column (a) below in Table 3 are obtainable to those skilled in the art:

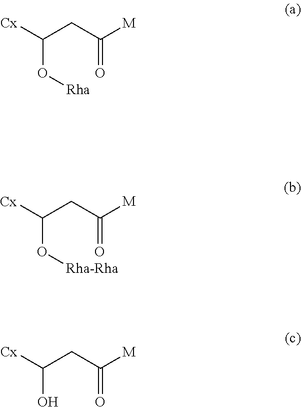

TABLE 3

| Entry | Reagent | Structure (a) | Structure (c) | Name (a) |
|---|---|---|---|---|
| 1 | CH3O−Na+ | 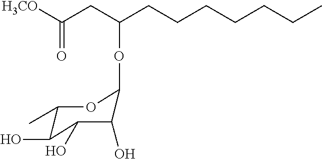 [SMILES: CCCCCCCC(CC(=O)OC)OC1OC(C)C(O)C(O)C1O] | | methyl 3-(((2R,3R,4R,5R,6S)-3,4,5- trihydroxy-6-methyltetrahydro-2H-pyran-2- yl)oxy)decanoate |
| 2 | iPrO−Na+ | 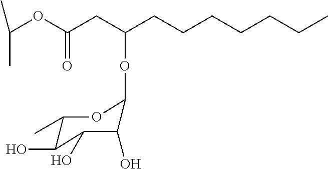 [SMILES: CCCCCCCC(CC(=O)OC(C)C)OC1OC(C)C(O)C(O)C1O] | | isopropyl 3-(((2R,3R,4R,5R,6S)-3,4,5- trihydroxy-6-methyltetrahydro-2H-pyran-2- yl)oxy)decanoate |
| 3 | NH(CH3)2 | 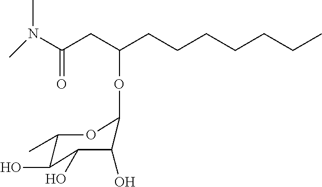 [SMILES: CCCCCCCC(CC(=O)N(C)C)OC1OC(C)C(O)C(O)C1O] | | N,N-dimethyl- 3-(((2R,3R,4R,5R,6S)-3,4,5- trihydroxy-6-methyltetrahydro-2H-pyran-2- yl)oxy)decanamide |
| 4 | Ethanolamine | 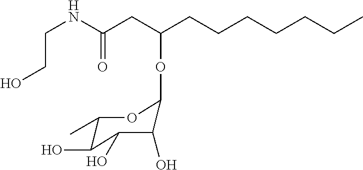 [SMILES: CCCCCCCC(CC(=O)NCCO)OC1OC(C)C(O)C(O)C1O] | | N-(2-hydroxyethyl)-3-(((2R,3R,4R,5R,6S)- 3,4,5-trihydroxy-6-methyltetrahydro-2H- pyran-2-yl)oxy)decanamide |
| 5 | taurine | 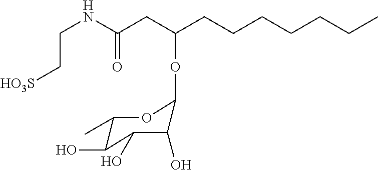 [SMILES: CCCCCCCC(CC(=O)NCCS(=O)(=O)O)OC1OC(C)C(O)C(O)C1O] | | 2-(3-(((2R,3R,4R,5R,6S)-3,4,5-trihydroxy-6- methyltetrahydro-2H-pyran-2- yl)oxy)decanamido)ethane-1-sulfonic acid |
| 6 | Me−Mg+Br | 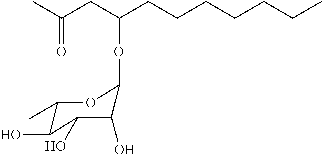 [SMILES: CCCCCCCC(CC(C)=O)OC1OC(C)C(O)C(O)C1O] | | 4-(((2R,3R,4R,5R,6S)-3,4,5-trihydroxy-6- methyltetrahydro-2H-pyran-2- yl)oxy)undecan-2-one |
| 7 | Bn−Mg+Cl | 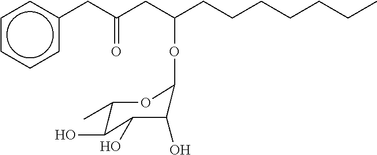 | | 1-phenyl-4-(((2R,3R,4R,5R,6S)-3,4,5- trihydroxy-6-methyltetrahydro-2H-pyran-2- yl)oxy)undecan-2-one |
| 8 | NH2(CH3) | 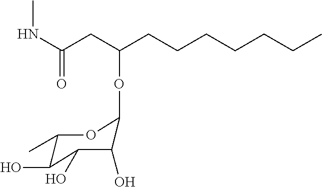 [SMILES: CCCCCCCC(CC(=O)NC)OC1OC(C)C(O)C(O)C1O] | | N-methyl-3-(((2R,3R,4R,5R,6S)-3,4,5- trihydroxy-6-methyltetrahydro-2H-pyran-2- yl)oxy)decanamide |
| 9 | benzylamine | 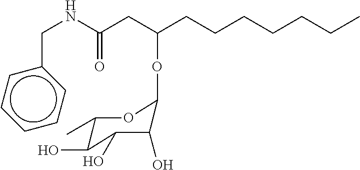 | | N-benzyl-3-(((2R,3R,4R,5R,6S)-3,4,5- trihydroxy-6-methyltetrahydro-2H-pyran-2- yl)oxy)decanamide |
| 10 | Glycine methyl ester | 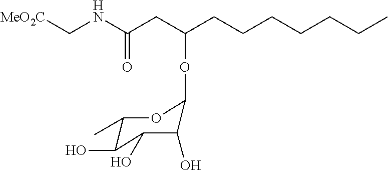 [SMILES: CCCCCCCC(CC(=O)NCC(=O)OC)OC1OC(C)C(O)C(O)C1O] | | methyl (3-(((2R,3R,4R,5R,6S)-3,4,5- trihydroxy-6-methyltetrahydro-2H-pyran-2- yl)oxy)decanoyl)glycinate |
| 11 | iPr−Mg+Br | 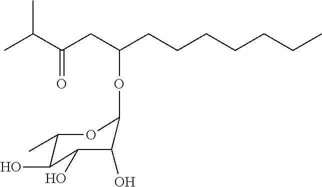 [SMILES: CCCCCCCC(CC(=O)C(C)C)OC1OC(C)C(O)C(O)C1O] | | 2-methyl-5-(((2R,3R,4R,5R,6S)-3,4,5- trihydroxy-6-methyltetrahydro-2H-pyran-2- yl)oxy)dodecan-3-one |

### Example O

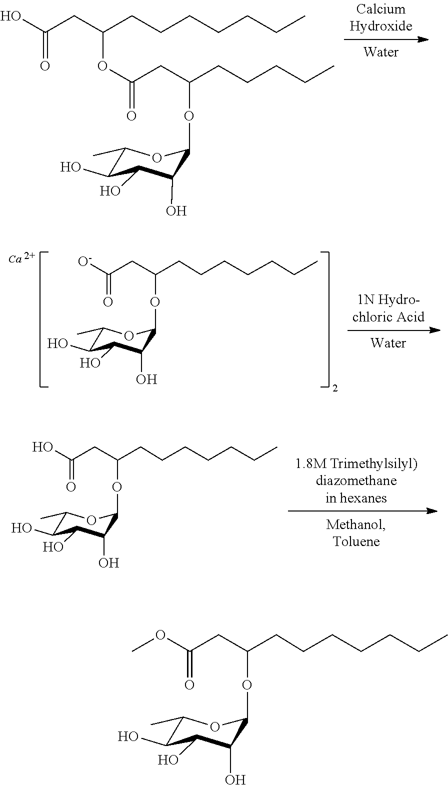

### Production of 3-(((2R,3R,4R,5R,6S)-3,4,5-trihydroxy-6-methyltetrahydro-2H-pyran-2-yl)oxy)decanoic acid

To a 100-ml, single neck, round bottom reaction flask equipped with a magnetic stir bar was added 5.03 grams (10 millimoles) of 3-((3-(((2R,3R,4R,5R,6S)-3,4,5-trihydroxy-6-methyltetrahydro-2H-pyran-2-yl)oxy)decanoyl)oxy)decanoic acid (Rha-C10C10 (racemic mixture), Glycosurf), and dissolved in 60 mL deionized water by heating to 87° C. Once the solution was homogeneous, 1.9 grams (26 millimoles) calcium hydroxide (1305-62-0, Sigma Aldrich) was added. Attached to the reaction flask was a water-cooled condenser equipped with a nitrogen line at the top leading to a gas bubbler using mixing under a nitrogen atmosphere, the reaction flask was heated for 5 hours at 87° C. in an oil bath. The reaction cooled to room temperature; at which time the reaction was filtered through a course glass fritted funnel, washed the filtered solids with 2×50 mL deionized water and the aqueous filtrate frozen and lyophilized, yielding 1.43 grams of a white solid after lyophilization. To a 500 mL Erlenmeyer flask, 0.95 grams of the lyophilized solids were added, dissolved in 160 mL deionized water. This solution was measured to be pH 12.2 using a pH meter. Adjusted pH of the solution to 3.5 with the dropwise addition of 1 N hydrochloric Acid (VWR Chemicals Product #BDH7202-1). The acidified solution was extracted 3×100 mL ethyl acetate (EMD Millipore Product #EX0240-6), the combined ethyl acetate extracts dried over sodium sulfate (Fisher Chemical Product #S429), filtered to remove the sodium sulfate. Evaporation of the filtrate was taken to completeness using a rotary evaporator with the water bath set at 20° C. and further dried under high vacuum (10-2 mBar) overnight to yield a light brown oil (525 milligrams, 58%) that was used without further purification.

LC/MS: m/z calc. for C16H29O7⁻ [M−1]⁻: 333.19 m/z, found 333.2 m/z

### Production of Methyl 3-(((2R,3R,4R,5R,6S)-3,4,5-trihydroxy-6-methyltetrahydro-2H-pyran-2-yl)oxy)decanoate

To a 15-ml, single neck, round bottom reaction flask was added 0.525 grams (0.0016 moles) of 3-(((2R,3R,4R,5R,6S)-3,4,5-trihydroxy-6-methyltetrahydro-2H-pyran-2-yl)oxy)decanoic acid, 2 ml of methanol (Fisher Chemical Product #A412-500), 3 mL of anhydrous toluene (Sigma Aldrich Product #244511) and a magnetic stir bar. Stirred reaction and dropwise added 2.2 mL (0.00396 moles) of 1.8M (Trimethylsilyl)diazomethane in hexanes (Thermoscientific Product #385330250) over a nitrogen atmosphere. Stirred reaction for 50 minutes at room temperature. The reaction was quenched by dropwise addition of glacial acetic acid (EMD Millipore Product #AX0073-6) until gas evolution ceased. Reaction solvents were removed by rotary evaporation and the remaining residue dried under high vacuum (10-2 mBar). The crude residue was purified via column chromatography (Biotage 40S Silica gel column), eluting with 5% methanol (Fisher Chemical Product #A412-500) in chloroform (EMD Millipore Product #CX1054-1) with 0.1% glacial acetic acid (EMD Millipore Product #AX0073-6) to yield a colorless oil (359 milligrams, 66% yield).

LC/MS: m/z calc. for C17H33O7⁺ [M+1]⁺: 349.22 m/z, found 349.2 m/z. (Table 3 (a) 1)

Possible bases may include sodium hydroxide (NaOH), potassium hydroxide (KOH), calcium hydroxide (CaOH), cesium hydroxide (CsOH), magnesium hydroxide (MgOH, ammonium hydroxide (NH4OH), and alkylamine containing bases such as triethylamine (Et3N).

TABLE 4
Antimicrobial Effect of Derivatized Rhamnolipids

| | | | E. coli | S. aureus |
|---|---|---|---|---|
| Structure | Sample Name | Solvent | MKC (ppm) | MKC (ppm) |
| 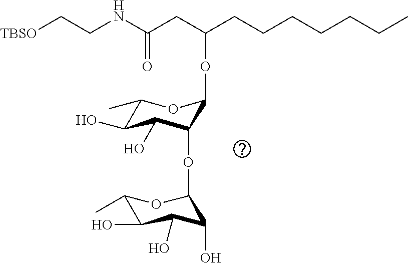 | N-(2-((tert- butyldimethylsilyl)oxy)ethyl)- 3-(((2R,3R,4R,5R,6S)-4,5- dihydroxy-6-methyl-3- (((2S,3R,4R,5R,6S)-3,4,5- trihydroxy-6- methyltetrahydro-2H-pyran-2- yl)oxy)tetrahydro-2H-pyran-2- yl)oxy)decanamide | DMSO | >6373.9 | >6373.9 |
| 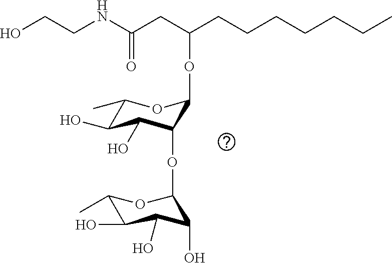 | 3-(((2R,3R,4R,5R,6S)-4,5- dihydroxy-6-methyl-3- (((2S,3R,4R,5R,6S)-3,4,5- trihydroxy-6- methyltetrahydro-2H-pyran-2- yl)oxy)tetrahydro-2H-pyran-2- yl)oxy)-N-(2- hydroxyethyl)decanamide | DMSO | >5236.2 | >5236.2 |
| 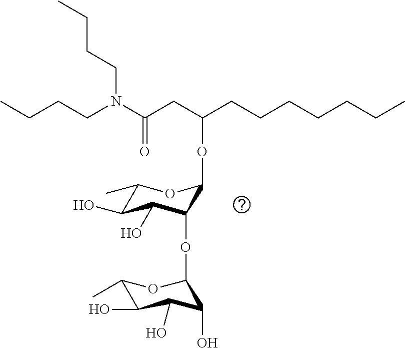 | N,N-dibutyl-3- (((2R,3R,4R,5R,6S)-4,5- dihydroxy-6-methyl-3- (((2S,3R,4R,5R,6S)-3,4,5- trihydroxy-6- methyltetrahydro-2H-pyran-2- yl)oxy)tetrahydro-2H-pyran-2- yl)oxy)decanamide | DMSO | 5917.8 | 2958.9 |
| 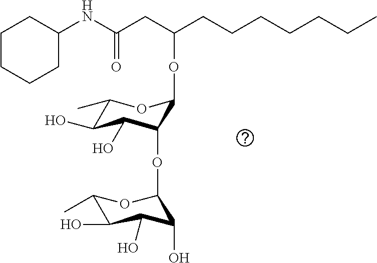 | N-cyclohexyl-3- (((2R,3R,4R,5R,6S)-4,5- dihydroxy-6-methyl-3- (((2S,3R,4R,5R,6S)-3,4,5- trihydroxy-6- methyltetrahydro-2H-pyran-2- yl)oxy)tetrahydro-2H-pyran-2- yl)oxy)decanamide | DMSO | >5617.1 | >5617.1 |
| 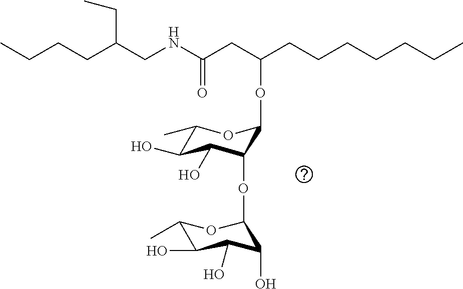 | 3-(((2R,3R,4R,5R,6S)-4,5- dihydroxy-6-methyl-3- (((2S,3R,4R,5R,6S)-3,4,5- trihydroxy-6- methyltetrahydro-2H-pyran-2- yl)oxy)tetrahydro-2H-pyran-2- yl)oxy)-N-(2- ethylhexyl)decanamide | DMSO | >5917.8 | 2958.9 |
| 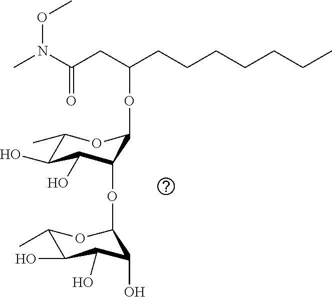 | 3-(((2R,3R,4R,5R,6S)-4,5- dihydroxy-6-methyl-3- (((2S,3R,4R,5R,6S)-3,4,5- trihydroxy-6- methyltetrahydro-2H-pyran-2- yl)oxy)tetrahydro-2H-pyran-2- yl)oxy)-N-methoxy-N- methyldecanamide | DMSO | >5236.2 | >5236.2 |
| 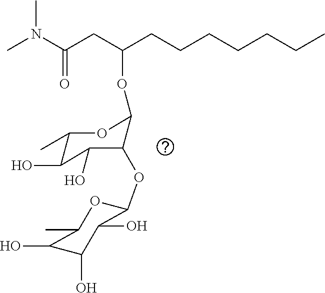 [SMILES: CCCCCCCC(CC(=O)N(C)C)OC1OC(C)C(O)C(O)C1OC1OC(C)C(O)C(O)C1O] | 3-(((2R,3R,4R,5R,6S)-4,5- dihydroxy-6-methyl-3- (((2S,3R,4R,6R)-3,4,5- trihydroxy-6- methyltetrahydro-2H-pyran-2- yl)oxy)tetrahydro-2H-pyran-2- yl)oxy)-N,N- dimethyldecanamide | DMSO | >5073.0 | >5073.0 |
| 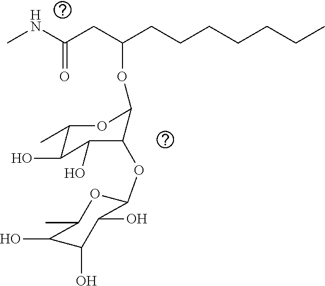 [SMILES: CCCCCCCC(CC(=O)NC)OC1OC(C)C(O)C(O)C1OC1OC(C)C(O)C(O)C1O] | Methyl 3-(((2R,3R,4R,5R,6S)- 4,5-dihydroxy-6-methyl-3- (((2R,3S,4S,5S,6S)-3,4,5- trihydroxy-6- methyltetrahydro-2H-pyran-2- yl)oxy)tetrahydro-2H-pyran-2- yl)oxy)decanoate | DMSO | >4945.8 | >4945.8 |
| 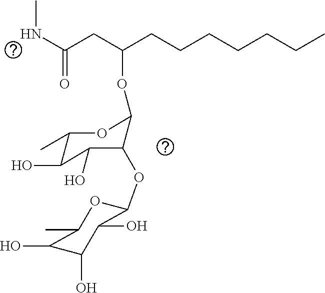 [SMILES: CCCCCCCC(CC(=O)NC)OC1OC(C)C(O)C(O)C1OC1OC(C)C(O)C(O)C1O] | 3-(((2R,3R,4R,5R,6S)-4,5- dihydroxy-6-methyl-3- (((2R,3S,4S,5S,6S)-3,4,5- trihydroxy-6- methyltetrahydro-2H-pyran-2- yl)oxy)tetrahydro-2H-pyran-2- yl)oxy)-N-methyldecanamide | DMSO | >4935.9 | >4935.9 |
| 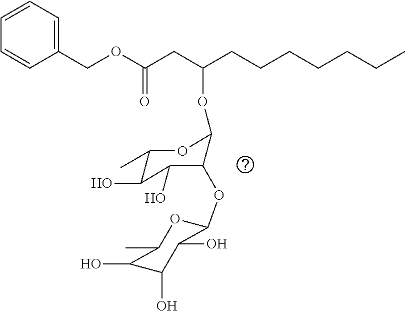 [SMILES: CCCCCCCC(CC(=O)OCc1ccccc1)OC1OC(C)C(O)C(O)C1OC1OC(C)C(O)C(O)C1O] | N-benzyl-3-(((2R,3R,4R,5R,6S)- 4,5-dihydroxy-6-methyl-3- (((2R,3S,4S,5S,6S)-3,4,5- trihydroxy-6- methyltetrahydro-2H-pyran-2- yl)oxy)tetrahydro-2H-pyran-2- yl)oxy)decanamide | DMSO | >5696.9 | >5696.9 |
| 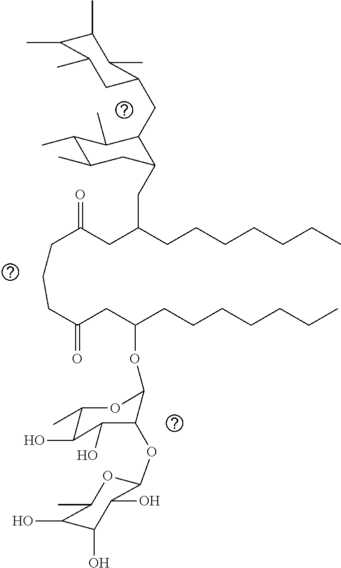 [SMILES: CCCCCCCC(CC(=O)CCCC(=O)CC(CCCCCCC)OC1OC(C)C(O)C(O)C1OC1OC(C)C(O)C(O)C1O)CC1CC(C)C(C)C(C)C1CC1CC(C)C(C)C(C)C1C] | calcium 3-(((2R,3R,4R,5R,6S)- 4,5-dihydroxy-6-methyl-3- (((2S,3R,4R,6R)-3,4,5- trihydroxy-6- methyltetrahydro-2H-pyran-2- yl)oxy)tetrahydro-2H-pyran-2- yl)oxy)decanoate | DMSO | >9392.1 | >9392.1 |
| 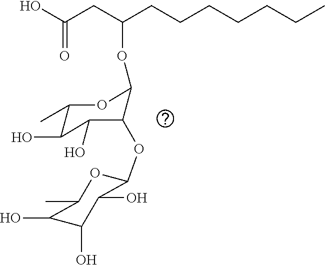 [SMILES: CCCCCCCC(CC(=O)O)OC1OC(C)C(O)C(O)C1OC1OC(C)C(O)C(O)C1O] | 3-(((2R,3R,4R,5R,6S)-4,5- dihydroxy-6-methyl-3- (((2S,3R,4R,6R)-3,4,5- trihydroxy-6- methyltetrahydro-2H-pyran-2- yl)oxy)tetrahydro-2H-pyran-2- yl)oxy)decanoic acid | DMSO | >4805.5 | >4805.5 |
| 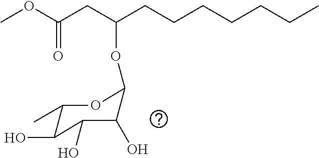 [SMILES: CCCCCCCC(CC(=O)OC)OC1OC(C)C(O)C(O)C1O] | methyl 3-(((2R,3R,4R,5R,6S)- 3,4,5-trihydroxy-6- methyltetrahydro-2H-pyran-2- yl)oxy)decanoate | DMSO | >3488.4 | >3488.4 |

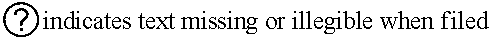

For information on the methodology, see 2. Minimal Kill Concentration Assay (MKCA) in the Test Methods section.

TABLE 5

| | | | | S. aureus |
|---|---|---|---|---|
| | | | S. aureus | MBC |
| Structure | Sample Name | Solvent | MIC (ppm) | (ppm) |
| 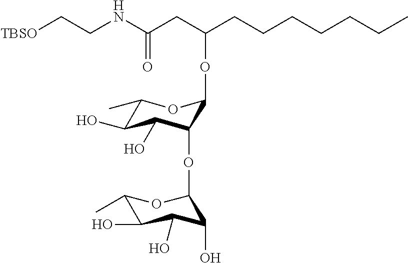 | N-(2-((tert- butyldimethylsilyl)oxy)ethyl)- 3-(((2R,3R,4R,5R,6S)-4,5- dihyroxy-6-methyl-3- (((2S,3R,4R,5R,6S)-3,4,5- trihydroxy-6- methyltetrahydro-2H-pyran-2- yl)oxy)tetrahydro-2H-pyran-2- yl)oxy)decanamide | DMSO | 318.7 | 637.4 |
| 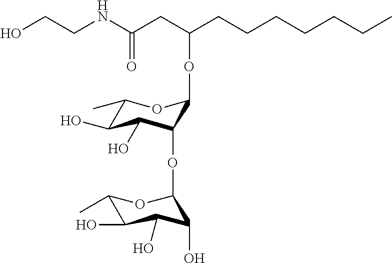 | 3-(((2R,3R,4R,5R,6S)-4,5- dihydroxy-6-methyl-3- (((2S,3R,4R,5R,6S)-3,4,5- trihydroxy-6- methyltetrahydro-2H-pyran-2- yl)oxy)tetrahydro-2H-pyran-2- yl)oxy)-N-(2- hydroxyethyl)decanamide | DMSO | >2094.5 | >2094.5 |
| 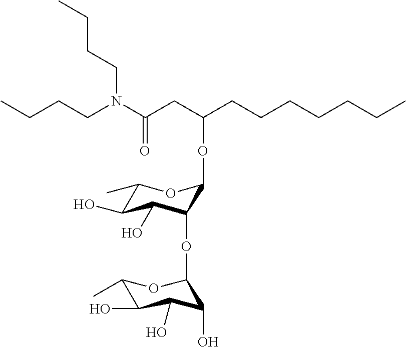 | N,N-dibutyl-3- (((2R,3R,4R,5R,6S)-4,5- dihydroxy-6-methyl-3- (((2S,3R,4R,5R,6S)-3,4,5- trihydroxy-6- methyltetrahydro-2H-pyran-2- yl)oxy)tetrahydro-2H-pyran-2- yl)oxy)decanamide | DMSO | 295.9 | 295.9 |
| 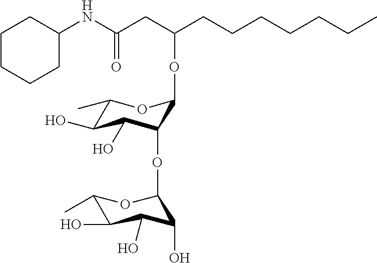 | N-cyclohexyl-3- (((2R,3R,4R,5R,6S)-4,5- dihydroxy-6-methyl-3- (((2S,3R,4R,5R,6S)-3,4,5- trihydroxy-6- methyltetrahydro-2H-pyran-2- yl)oxy)tetrahydro-2H-pyran-2- yl)oxy)decanamide | DMSO | >2246.8 | >2246.8 |
| 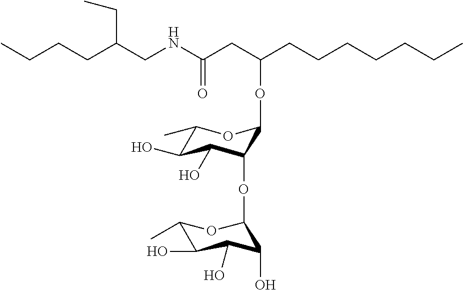 | 3-(((2R,3R,4R,5R,6S)-4,5- dihydroxy-6-methyl-3- (((2S,3R,4R,5R,6S)-3,4,5- trihydroxy-6- methyltetrahydro-2H-pyran-2- yl)oxy)tetrahydro-2H-pyran-2- yl)oxy)-N-(2- ethylhexyl)decanamide | DMSO | 295.9 | 591.8 |
| 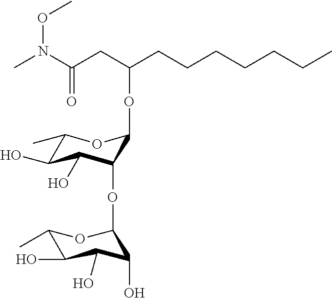 | 3-(((2R,3R,4R,5R,6S)-4,5- dihydroxy-6-methyl-3- (((2S,3R,4R,5R,6S)-3,4,5- trihydroxy-6- methyltetrahydro-2H-pyran-2- yl)oxy)tetrahydro-2H-pyran-2- yl)oxy)-N-methoxy-N- methyldecanamide | DMSO | 2094.5 | >2094.5 |
| 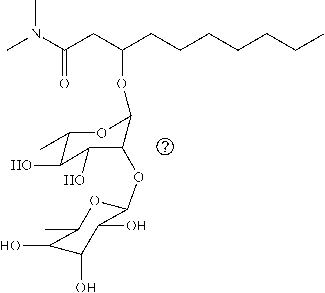 [SMILES: CCCCCCCC(CC(=O)N(C)C)OC1OC(C)C(O)C(O)C1OC1OC(C)C(O)C(O)C1O] | 3-(((2R,3R,4R,5R,6S)-4,5- dihydroxy-6-methyl-3- (((2S,3R,4R,6R)-3,4,5- trihydroxy-6- methyltetrahydro-2H-pyran-2- yl)oxy)tetrahydro-2H-pyran-2- yl)oxy)-N,N- dimethyldecanamide | DMSO | >507.5 | >507.5 |
| 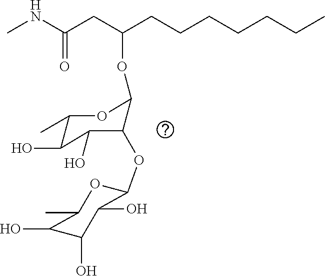 [SMILES: CCCCCCCC(CC(=O)NC)OC1OC(C)C(O)C(O)C1OC1OC(C)C(O)C(O)C1O] | Methyl 3-(((2R,3R,4R,5R,6S)- 4,5-dihydroxy-6-methyl-3- (((2R,3S,4S,5S,6S)-3,4,5- trihydroxy-6- methyltetrahydro-2H-pyran-2- yl)oxy)tetrahydro-2H-pyran-2- yl)oxy)decanoate | DMSO | 1978.3 | 1978.3 |
| 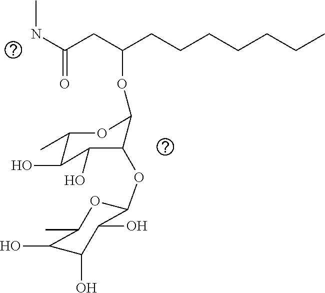 [SMILES: CCCCCCCC(CC(=O)NC)OC1OC(C)C(O)C(O)C1OC1OC(C)C(O)C(O)C1O] | 3-(((2R,3R,4R,5R,6S)-4,5- dihydroxy-6-methyl-3- (((2R,3S,4S,5S,6S)-3,4,5- trihydroxy-6- methyltetrahydro-2H-pyran-2- yl)oxy)tetrahydro-2H-pyran-2- yl)oxy)-N-methyldecanamide | DMSO | >1974.4 | >1974.4 |
| 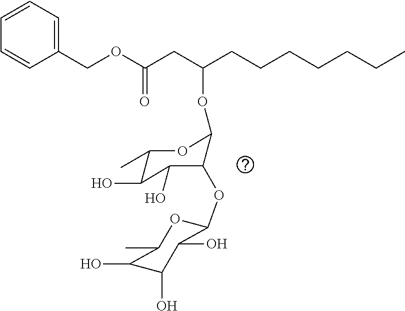 [SMILES: CCCCCCCC(CC(=O)OCc1ccccc1)OC1OC(C)C(O)C(O)C1OC1OC(C)C(O)C(O)C1O] | N-benzyl-3-(((2R,3R,4R,5R,6S)- 4,5-dihydroxy-6-methyl-3- (((2R,3S,4S,5S,6S)-3,4,5- trihydroxy-6- methyltetrahydro-2H-pyran-2- yl)oxy)tetrahydro-2H-pyran-2- yl)oxy)decanamide | DMSO | 2278.8 | >2278.3 |
| 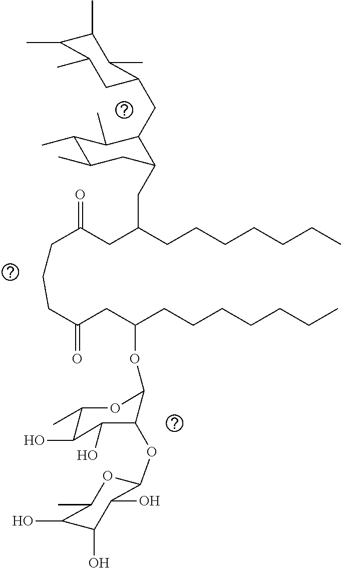 [SMILES: CCCCCCCC(CC(=O)CCCC(=O)CC(CCCCCCC)OC1OC(C)C(O)C(O)C1OC1OC(C)C(O)C(O)C1O)CC1CC(C)C(C)C(C)C1CC1CC(C)C(C)C(C)C1C] | calcium 3-(((2R,3R,4R,5R,6S)- 4,5-dihydroxy-6-methyl-3- (((2S,3R,4R,6R)-3,4,5- trihydroxy-6- methyltetrahydro-2H-pyran-2- yl)oxy)tetrahydro-2H-pyran-2- yl)oxy)decanoate | DMSO | >3996.6 | >3996.6 |
| 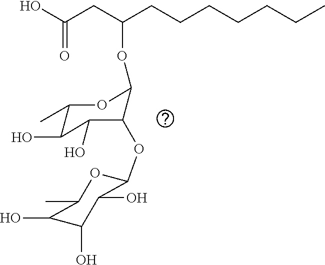 [SMILES: CCCCCCCC(CC(=O)O)OC1OC(C)C(O)C(O)C1OC1OC(C)C(O)C(O)C1O] | 3-(((2R,3R,4R,5R,6S)-4,5- dihydroxy-6-methyl-3- (((2S,3R,4R,6R)-3,4,5- trihydroxy-6- methyltetrahydro-2H-pyran-2- yl)oxy)tetrahydro-2H-pyran-2- yl)oxy)decanoic acid | DMSO | >1922.2 | >1922.2 |
| 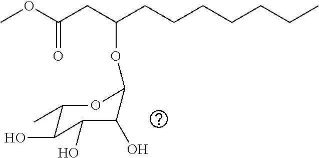 [SMILES: CCCCCCCC(CC(=O)OC)OC1OC(C)C(O)C(O)C1O] | methyl 3-(((2R,3R,4R,5R,6S)- 3,4,5-trihydroxy-6- methyltetrahydro-2H-pyran-2- yl)oxy)decanoate | DMSO | 1395.4 | 1395.4 |

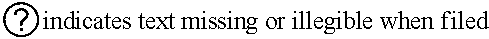

For information on the methodology, see 3. Minimum Inhibitory Concentration (MIC) Assay in the Test Methods section.

### Color

In some embodiments, the inventive rhamnolipid compositions may be made by reacting a rhamnolipid aqueous solution, such as Evonik Rheance One, with a base and/or caustic under various time conditions and/or various temperature conditions. The time condition, that is, how long the reaction may occur, may be anywhere from about 0.25 hours to about 100 hours. Independently, the temperature condition for the reaction may be from about 25° C. to about 100° C. After the reaction, the water may be removed by any means known in the art such as distillation under reduce pressure (a.k.a. vacuum distillation), and freeze-drying (or lyophilization).

In some cases, the temperature and/or the length of time of the reaction may affect the color of the resulting inventive compositions. As the data in Table 6 shows, temperature has the most significant effect on color formation, more so than the length of the reaction. Stoichiometry is the main factor impacting extent of hydrolysis reaction.

In some uses of the invention compositions, a lighter color may be preferred. Aiming to minimize color development during the hydrolysis of Rheance One resulting in inventive compositions, a two-factor Design of Experiment (DOX) with augmented points and repeat points was designed using JMP software (SAS company, Cary, NC, USA). Time (ranging from 1 to 8 hours), temperature (ranging from 35° C. to 55° C.), and stoichiometry molar ratios were used as the input variables. Color was measured using a spectrophotometric colorimeter and reported in various scales such as AOCS Red & Yellow, and Gardner. B-hydroxy fatty acids resulting from the hydrolysis were quantified via GC-FID. Eighteen experiments were carried out, with the test methods detailed below. Afterwards, an empirical model was generated using the same software. The model revealed that temperature has the most significant effect on color formation, while stoichiometry was the main factor impacting extent of hydrolysis reaction.

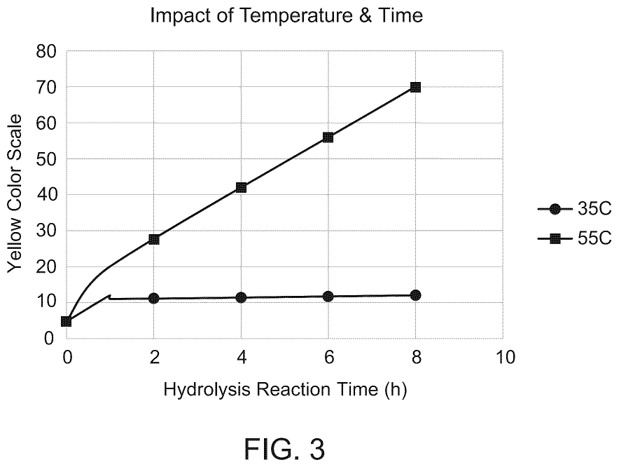

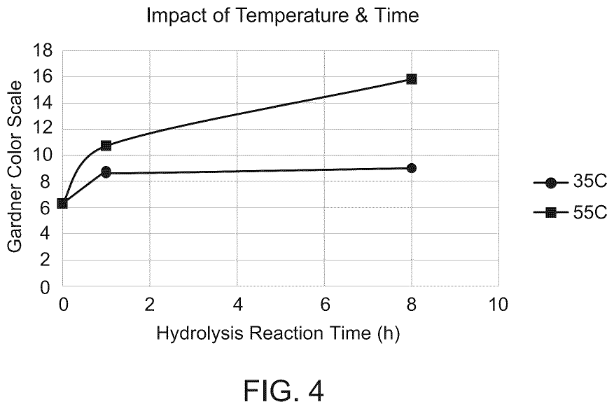

The impact of temperature on color formation is clear when plotting color data vs hydrolysis reaction time for a subset of the inventive reactions conducted at the same Rheance One to sodium hydroxide molar ratio but a different temperature. The data shows that a lower temperature results in lighter colored compositions. The data is shown in Table 6 and the graphs of the data are shown in FIGS. 3 and 4, with FIG. 3 showing color measured on the Yellow Color Scale, and FIG. 4 showing color measured on the Gardner Color Scale:

TABLE 6

| | | | | | | Total β- | |
|---|---|---|---|---|---|---|---|
| | | | | | | hydroxy | |
| DOX | | | | | | Fatty | |
| exp. | Temperature | Time | | | | Acid | Extent of |
| Leg# | (° C.) | (h) | Red | Yellow | Gardner | (wt. %) | Hydrolysis, % |
| Control | | 0 | 1.3 | 4.7 | 6.3 | — | |
| (Rheance | | | | | | | |
| One) | | | | | | | |
| 9 | 35 | 1 | 2.1 | 12 | 8.8 | 10.13 | 64.5 |
| 4 | 35 | 1 | 2.1 | 11 | 8.6 | 10.46 | 66.8 |
| 11 | 35 | 8 | 2.4 | 12 | 9 | 11.05 | 71.1 |
| 1 | 35 | 8 | 2.4 | 12 | 9 | 10.97 | 70.5 |
| 3 | 55 | 1 | 3.1 | 20 | 10.7 | 10.88 | 70.0 |
| 6 | 55 | 8 | 13.2 | 70 | 15.8 | 12.87 | 84.2 |

The inventive hydrolysates in Table 6, while not identical to the inventive composition in Example A, would be similarly rich in rhamnomonolipids. Mass spectroscopy would likely show that the rhamnodilipids of the starting material were highly hydrolyzed, meaning that the rhamnodilipids would be detected at significantly lower amounts. Table 6 shows the amount of beta hydroxy fatty acid formed from each hydrolysis, which was used to estimate the extent of hydrolysis. In addition, when evaluating lathering ability, the Table 6 hydrolysate generated at 35° C. for 1 hour, which has a lighter color than the hydrolysate of Example A, performed similarly to Example A.

To further reduce (lighten) the color of the inventive hydrolysate obtained from the hydrolysis at the reaction conditions of 35° C. for 1 hour and a molar ratio of 1 to 2.25 starting material to base, a two-factor Design of Experiment (DOX) with augmented points and repeat points was designed using JMP software. Time (ranging from 1 to 8 hours) and hydrogen peroxide concentration (ranging from 0.05 to 0.50 wt. % on hydrolysate) were used as the input variables, the decolorization treatments were conducted at 35° C. Color was measured using a spectrophotometric colorimeter and reported in various scales such as AOCS Red & Yellow, and Gardner.

Decolorization ratio was calculated using the equation below:

$${D}_{r} = \frac{{G}_{o} - {G}_{1}}{{G}_{o}} \times 100$$

where G₀ and G₁ are the Gardner color values of the hydrolysate before and after decolorization respectively, and Dᵣ is the decolorization ratio (percent reported).

The results in Table 7 show that hydrolysate treated for 1 hour (h) at 35° C. with as little as 0.05 wt % of H₂O₂ is effective for decolorizing hydrolysate to a similar color profile as the starting Rheance One (experiments #s 36 and 39). Extending the reaction to 8h at the same peroxide concentration does not improve the color (experiments #s 37 & 40). Increasing H₂O₂ concentration to 0.16 and 0.22 wt % further improves the color (experiments #s 42 & 43).

TABLE 7

| | | | 50% | | | | | | |
|---|---|---|---|---|---|---|---|---|---|
| DOX | Target | Hydrolysate | H₂O₂ | | Mixing | | | | Decolorization |
| Exp. | H₂O₂ | (actual, | (actual, | Temperature, | Time | | | | Ratio |
| Leg # | (wt. %) | g) | mg) | (° C.) | (h) | Red | Yellow | Gardner | (%) |
| Hydrolysate | — | — | — | — | — | 2.1 | 11.5 | 8.7 | Control |
| 33 | 0.50% | 10.0329 | 103.30 | 35 | 1 | 0.4 | 2 | 4.3 | 50.6 |
| 34 | 0.50% | 10.1301 | 106.40 | 35 | 8 | 0.5 | 2.1 | 4.4 | 49.4 |
| 35 | 0.50% | 10.0010 | 113.50 | 35 | 1 | 0.3 | 2.1 | 4.4 | 49.4 |
| 36 | 0.05% | 10.0206 | 17.40 | 35 | 1 | 0.9 | 4.5 | 6.3 | 27.6 |
| 37 | 0.05% | 10.0475 | 16.80 | 35 | 8 | 0.9 | 4.4 | 6.2 | 28.7 |
| 38 | 0.50% | 10.0081 | 103.30 | 35 | 8 | 0.3 | 1.6 | 3.9 | 55.2 |
| 39 | 0.05% | 10.0230 | 14.40 | 35 | 1 | 1 | 5 | 6.4 | 26.4 |
| 40 | 0.05% | 10.0159 | 17.50 | 35 | 8 | 0.9 | 4.2 | 6.1 | 29.9 |
| 41 | 0.31% | 10.0212 | 61.20 | 35 | 3 | 0.3 | 2.3 | 4.7 | 46.0 |
| 42 | 0.16% | 10.1129 | 29.00 | 35 | 2 | 0.6 | 3.4 | 5.7 | 34.5 |
| 43 | 0.22% | 10.0209 | 47.10 | 35 | 6 | 0.4 | 2.4 | 4.8 | 44.8 |
| 44 | 0.41% | 10.0047 | 82.10 | 35 | 5 | 0.3 | 1.8 | 4.1 | 52.9 |

### Antimicrobial or Preservative

The inventive compositions of the present invention may be used as part of a personal care composition or a detergent composition. In some embodiments the inventive compositions may have effect as a surfactant and/or as an antimicrobial and/or as a preservative. In some embodiments, the present invention may be an antimicrobial product comprising the inventive composition. In some embodiments, the present invention may be a personal care product or composition comprising the inventive composition. In some embodiments, the present invention may be a method of reducing or killing bacteria by using or comprising the inventive composition. In some embodiments, such methods may be used to reduce microbial population of formulated products, biological tissue or inanimate materials. In some embodiments, the formulated products, biological tissue, or inanimate materials may contact the inventive composition for sufficient time to provide substantial microbial reduction. The amount of inventive composition in a personal care composition, a detergent composition, a preservative composition, or an antimicrobial composition may be from about 0.1 to about 25% by weight of the composition, from about 0.2 to about 15% by weight of the composition, or from 0.5 to about 12% of the composition.

### Personal Care Composition

The inventive compositions described herein may be used, for example, in personal care compositions or detergent compositions. They may be used as surfactants with or without additional surfactants, such as, optionally, one or more additional non-sulfated surfactants and/or other ingredients commonly found in compositions of the type described. The personal care compositions herein may be provided in various product forms such as solutions, suspensions, shampoos, conditioners, lotions, creams, gels, toners, sticks, sprays, aerosols, ointments, cleansing liquid washes, solid bars, pastes, foams, mousses, shaving creams, wipes, strips, patches, hydrogels, film-forming products, facial and skin masks (with and without insoluble sheet), and the like. The composition form may follow from the particular dermatologically acceptable carrier chosen. In some aspects, the personal care compositions described herein may include a dispersed gel network phase that provides a milder, but effective, cleansing benefit to soiled hair in combination with a detersive glycolipid surfactant.

### Method of Making a Personal Care Composition

The personal care compositions described herein can be made using conventional methods for making compositions of the type desired (e.g., shampoo, conditioner or body wash). In some specific gel network containing aspects, the composition may be made by: (a) combining a fatty alcohol, a gel network surfactant, and water at a temperature sufficient to allow partitioning of the secondary surfactant and the water into the fatty alcohol to form a pre-mix; (b) cooling the pre-mix below the chain melt temperature of the fatty alcohol to form a gel network; (c) adding the gel network to one or more detersive surfactants and a liquid carrier to form a personal care composition which includes a dispersed gel network phase having a melt transition temperature of at least about 38° C.

In some aspects, the gel network phase can be prepared by heating the fatty alcohol, the gel network surfactant, and water to a level in the range of about 75° C. to about 90° C. and mixing. This mixture can be cooled to 27-35° C. (e.g., by passing the mixture through a heat exchanger). As a result of this cooling step, at least about fifty percent of the mixture of the fatty alcohol and the gel network surfactant crystallize to form a crystalline gel network.

Other methods of preparing the gel network phase include sonication and/or milling of the fatty alcohol, the gel network surfactant, and water, while these components are heated, to reduce the particle size of the dispersed gel network phase. This results in an increase in surface area of the gel network phase, which allows the gel network surfactant and the water to swell the gel network phase. Another variation in preparing the gel network includes heating and mixing the fatty alcohol and the gel network surfactant first, and then adding that mixture to the water.

Tables 8 and 9 show personal cleansing compositions.

Comparative Personal Cleansing Composition 1 can be formed by the following process. DI water is added to a mixing vessel and heated to 75° C.±3° C. while agitating. Sodium Cocoyl Isethionate (SCI) is added to the mixing vessel, and the mixing continues until the SCI has fully dissolved (with no visible particles remaining and batch is clear). After the SCI has fully dissolved, the following materials are added to the mixing vessel: Alkyl Amidopropyl Betaine and Sodium Lauroyl Sarcosinate. The vessel contents are mixed for at least 10 minutes. The batch is then cooled to <35° C. A Polyquaternium-10 slurry is made with water, which is immediately added to the mixing vessel and mixed for 10 minutes. Perfume is then added and mixed in the mixture for at least 2 minutes. Citric Acid is used to titrate the mixture until a pH of 5.5 to 6.0 is reached. DI water is added to bring the final volume to 100%. The mixture is mixed for at least 10 minutes until homogeneity is achieved.

Comparative Personal Cleansing Compositions 2 can be formed by the following process. DI water is added to a mixing vessel while agitating. The following material is then added to the mixing vessel: Alkyl Amidopropyl Betaine. The vessel contents are mixed for at least 10 minutes. A Poly quaternium-10 slurry is made with water, which is immediately added to the mixing vessel and mixed for 10 minutes. Perfume is then added and mixed in the mixture for at least 2 minutes. Citric Acid is used to titrate the mixture until a pH of 5.5 to 6.0 is reached. DI water is added to bring the final volume to 100%. The mixture is mixed for at least 10 minutes until homogeneity is achieved.

Comparative Personal Cleansing Compositions 3-5 and Inventive Personal Cleansing Compositions 1-4 can be formed by the following process. Deionized water is added to a mixing vessel while agitating. The following materials are then added to the mixing vessel: Alkyl Amidopropyl Betaine and Rhamnolipids. The vessel contents are mixed for at least 10 minutes. A Polyquaternium-10 slurry is made with water, which is immediately added to the mixing vessel and mixed for 10 minutes. Perfume is then added and mixed in the mixture for at least 2 minutes. Citric acid is used to titrate the mixture until a pH of 6.8 to 7.2 is reached. Deionized water is added to bring the final volume to 100%. The mixture is mixed for at least 10 minutes until homogeneity is achieved.

The Inventive Examples, comprising the inventive hydrolyzed rhamnolipids cocktail, have significant lather volume and creaminess improvements over the Comparative Examples that comprise commercial rhamnolipid controls when rhamnolipids are used as the primary anionic surfactant in sulfate-free shampoo formulations.

At high levels (8.5%), the inventive compositions comprising rhamnolipid hydroly sates provide conditioning benefits versus the commercially available rhamnolipids (e.g., “Lather Combing”, “Slippery Feel While Rinsing”, “Ease of Combing Post Rinsing”).

Sulfate-free shampoo formulations with 8.5% inventive hydrolyzed rhamnolipid mixture display broad-spectrum antimicrobial efficacy against bacteria, yeast, and mold.

TABLE 8

| | Comparative | Comparative | Comparative | Comparative | Comparative |
|---|---|---|---|---|---|
| | Example 1 | Example 2 | Example 3 | Example 4 | Example 5 |
| | Sulfate Free | Sulfate Free | Commercial | Commercial | Commercial |
| | Composition | Composition | Rhamnolipids | Rhamnolipids | Rhamnolipids |
| | (wt. %) | (wt. %) | (wt. %) | (wt. %) | (wt. %) |
| Alkyl | 9.8 | 9.8 | 9.8 | 9.8 | 9.8 |
| Amidopropyl | | | | | |
| Betaine¹ | | | | | |
| Sodium Cocoyl | 6.0 | — | — | — | — |
| Isethionate | | | | | |
| Sodium Lauroyl | 2.5 | — | — | — | — |
| Sarcosinate | | | | | |
| Mono-sugar/di- | — | — | 8.5 | — | — |
| lipid + Di-sugar/di- | | | | | |
| lipid Rhamnolipids | | | | | |
| mixture I ² | | | | | |
| Mono-sugar/di- | — | — | — | 8.5 | — |
| lipid + | | | | | |
| Di-sugar/di-lipid | | | | | |
| Rhamnolipids | | | | | |
| mixture II ³ | | | | | |
| Mono-sugar/di- | — | — | — | — | 8.5 |
| lipid + | | | | | |
| Di-sugar/di-lipid | | | | | |
| Rhamnolipids | | | | | |
| mixture III ⁴ | | | | | |
| Mono-sugar/mono- | — | — | — | — | — |
| lipid + Di- | | | | | |
| sugar/mono-lipid | | | | | |
| Rhamnolipids | | | | | |
| mixture I ⁵ | | | | | |
| Mono-sugar/mono- | — | — | — | — | — |
| lipid + Di- | | | | | |
| sugar/mono-lipid | | | | | |
| Rhamnolipids | | | | | |
| mixture II ⁶ | | | | | |
| Mono-sugar/mono- | — | — | — | — | — |
| lipid + Di- | | | | | |
| sugar/mono-lipid | | | | | |
| Rhamnolipids | | | | | |
| mixture III ⁷ | | | | | |
| Mono-sugar/mono- | — | — | — | — | — |
| lipid + Di- | | | | | |
| sugar/mono-lipid | | | | | |
| Rhamnolipids | | | | | |
| mixture IV ⁸ | | | | | |
| Polyquaternium-10 | 0.3 | 0.3 | 0.3 | 0.3 | 0.3 |
| Perfume | 1.1 | 1.1 | 1.1 | 1.1 | 1.1 |
| Buffer | To pH 5.5 | To pH 5.5 | To pH 6.8 | To pH 6.8 | To pH 6.8 |
| | to 6.0 | to 6.0 | to 7.2 | to 7.2 | to 7.2 |
| Water | qs | qs | qs | qs | qs |
| TECHNICAL DATA ⁹ | | | | | |
| Lather Blender | 8.3 | 3.0 | 2.5 | 2.0 | 2.5 |
| Height (w/oil) | | | | | |
| (cm) * | | | | | |
| Lather Blender | 4 | 0 | 0 | 0 | 0 |
| Creaminess Rating | | | | | |
| (0-5; 0 = not at all | | | | | |
| creamy, | | | | | |
| 5 = extremely | | | | | |
| creamy) | | | | | |
| Lather Blender | 15.2 | 13.0 | 13.0 | 11.0 | 13.0 |
| Height (w/dirt) | | | | | |
| (cm) * | | | | | |
| “Slippery Feel at | 8 | 3 | 6 | 6 | 6 |
| application” wet | | | | | |
| hair ILS rating | | | | | |
| “Speed to Lather” | 7 | 5 | 7 | 6 | 6 |
| wet hair ILS rating | | | | | |
| “Lather Combing” | 5 | 1 | 2 | — | — |
| wet hair ILS rating ** | | | | | |
| “Slippery Feel | 6 | 3 | 5 | 5 | 4 |
| While Rinsing” | | | | | |
| wet hair ILS rating ** | | | | | |
| “Slippery Feel Post | 5 | 1 | 3 | 2 | 3 |
| Rinsing” wet hair | | | | | |
| ILS rating | | | | | |
| “Ease of | 7 | 1 | 2 | 1 | 1 |
| Combing” wet hair | | | | | |
| ILS rating ** | | | | | |
| MST Log | <0.2 | <0.2 | <0.2 | <0.2 | <0.2 |
| reduction: Bacteria | | | | | |
| Pool (Day 2) | | | | | |
| (antimicrobial | | | | | |
| activity) | | | | | |
| MST Log | <0.2 | 0.2 | <0.2 | <0.2 | <0.2 |
| reduction: Bacteria | | | | | |
| Pool (Day 7) | | | | | |
| (antimicrobial | | | | | |
| activity) | | | | | |
| MST Log | 0.2 | <0.2 | 0.4 | <0.2 | <0.2 |
| reduction: | | | | | |
| Yeast/Mold Pool | | | | | |
| (Day 7) | | | | | |
| (antimicrobial | | | | | |
| activity) | | | | | |
| MST Log | 0.2 | 0.5 | 0.8 | 0.9 | 0.6 |
| reduction: | | | | | |
| Yeast/Mold Pool | | | | | |
| (Day 14) | | | | | |
| (antimicrobial | | | | | |
| activity) | | | | | |

¹Mixture of Chainlengths
² Evonik Rheance ® One rhamnolipids (~50% active), Evonik Industries AG, Essen, Germany
³ Wanhua Carfil ® Bio-RL1 rhamnolipids (~38% active), Wanhua Chemical Group Co., Ltd., Yantai, China
⁴ BioReNuva ReNuva RL-50 rhamnolipids (~50% active), BioReNuva, Austin, TX, USA
⁵ Hydrolysate of Evonik Rheance One rhamnolipids w/HCl neutralization & EtOAc extraction (~50% active)
⁶ Hydrolysate of Evonik Rheance One rhamnolipids w/o post-hydrolysis work-up (~50% active)
⁷ Hydrolysate of Wanhua Carfil Bio-RL1 rhamnolipids w/o post-hydrolysis work-up (~38% active)
⁸ Hydrolysate of BioReNuva ReNuva RL-50 rhamnolipids w/o post-hydrolysis work-up (~50% active)
⁹ (s) Significant difference is operator noticeable vs Comparative Example w/Commercial Rhamnolipids
* ≥2 cm difference is considered consumer noticeable
** ≥2 point difference is considered consumer noticeable
^{#} demonstrates robust bacteria and fungal reduction

TABLE 9

| | Inventive | Inventive | Inventive | Inventive |
|---|---|---|---|---|
| | Example 1 | Example 2 | Example 3 | Example 4 |
| | (wt. %) | (wt. %) | (wt. %) | (wt. %) |
| Alkyl Amidopropyl | 9.8 | 9.8 | 9.8 | 9.8 |
| Betaine¹ | | | | |
| Sodium Cocoyl | — | — | — | — |
| Isethionate | | | | |
| Sodium Lauroyl | — | — | — | — |
| Sarcosinate | | | | |
| Mono-sugar/di-lipid + | — | — | — | — |
| Di-sugar/di-lipid | | | | |
| Rhamnolipids mixture | | | | |
| I² | | | | |
| Mono-sugar/di-lipid + | — | — | — | — |
| Di-sugar/di-lipid | | | | |
| Rhamnolipids mixture | | | | |
| II ³ | | | | |
| Mono-sugar/di-lipid + | — | — | — | — |
| Di-sugar/di-lipid | | | | |
| Rhamnolipids mixture | | | | |
| III ⁴ | | | | |
| Mono-sugar/mono- | 8.5 | — | — | — |
| lipid + Di-sugar/mono- | | | | |
| lipid Rhamnolipids | | | | |
| mixture I ⁵ | | | | |
| Mono-sugar/mono- | — | 8.5 | — | — |
| lipid + Di-sugar/mono- | | | | |
| lipid Rhamnolipids | | | | |
| mixture II ⁶ | | | | |
| Mono-sugar/mono- | — | — | 8.5 | — |
| lipid + Di-sugar/mono- | | | | |
| lipid Rhamnolipids | | | | |
| mixture III ⁷ | | | | |
| Mono-sugar/mono- | — | — | — | 8.5 |
| lipid + Di-sugar/mono- | | | | |
| lipid Rhamnolipids | | | | |
| mixture IV ⁸ | | | | |
| Poly quaternium-10 | 0.3 | 0.3 | 0.3 | 0.3 |
| Perfume | 1.1 | 1.1 | 1.1 | 1.1 |
| Buffer | To pH 6.8 | To pH 6.8 | To pH 6.8 | To pH 6.8 |
| | to 7.2 | to 7.2 | to 7.2 | to 7.2 |
| Water | qs | qs | qs | qs |
| TECHNICAL DATA ⁹ | | | | |
| Lather Blender Height | 9.5 (s) | 8.0 (s) | 9.5 (s) | 9.0 (s) |
| (w/oil) (cm) * | | | | |
| Lather Blender | 5 (s) | 4 (s) | 5 (s) | 5 (s) |
| Creaminess Rating | | | | |
| (0-5; 0 = not at all | | | | |
| creamy, | | | | |
| 5 = extremely creamy) | | | | |
| Lather Blender Height | 16.5 (s) | 16.0 (s) | 17.0 (s) | 17.0 (s) |
| (w/dirt) (cm) * | | | | |
| “Slippery Feel at | 5 | 6 | 5 | 5 |
| application” wet hair | | | | |
| ILS rating | | | | |
| “Speed to Lather” wet | 7 | 7 | 6 | 5 |
| hair ILS rating | | | | |
| “Lather Combing” wet | 7 (s) | 4 (s) | — | — |
| hair ILS rating ** | | | | |
| “Slippery Feel While | 7 (s) | 5 | 4 | 4 |
| Rinsing” wet hair ILS | | | | |
| rating ** | | | | |
| “Slippery Feel Post | 4 | 3 | 2 | 3 |
| Rinsing” wet hair ILS | | | | |
| rating | | | | |
| “Ease of Combing” wet | 6 (s) | 2 | 1 | 2 |
| hair ILS rating ** | | | | |
| MST Log reduction: | ≥4.7 ^{#} | 2.7 ^{#} | >4.8 ^{#} | 4.4 ^{#} |
| BacteriaPool (Day 2) | | | | |
| (antimicrobial | | | | |
| activity) | | | | |
| MST Log reduction: | ≥4.7 ^{#} | 3.9 ^{#} | >4.8 ^{#} | >4.8 ^{#} |
| BacteriaPool (Day 7) | | | | |
| (antimicrobial | | | | |
| activity) | | | | |
| MST Log reduction: | 3.1 | 2.6 | >3.8 | >3.8 |
| Yeast/Mold Pool (Day | | | | |
| 7) (antimicrobial | | | | |
| activity) | | | | |
| MST Log reduction: | ≥3.7 ^{#} | 2.9 ^{#} | >3.8 ^{#} | >3.8 ^{#} |
| Yeast/Mold Pool (Day | | | | |
| 14) (antimicrobial | | | | |
| activity) | | | | |

¹Mixture of Chainlengths
²Evonik Rheance One rhamnolipids (~50% active)
³ Wanhua Carfil Bio-RL1 rhamnolipids (~38% active)
⁴ BioReNuva ReNuva RL-50 rhamnolipids (~50% active)
⁵ Hydrolysate of Evonik Rheance One rhamnolipids w/HCl neutralization & EtOAc extraction (~50% active)
⁶ Hydrolysate of Evonik Rheance One rhamnolipids w/o post-hydrolysis work-up (~50% active)
⁷ Hydrolysate of Wanhua Carfil Bio-RL1 rhamnolipids w/o post-hydrolysis work-up (~38% active)
⁸ Hydrolysate of BioReNuva ReNuva RL-50 rhamnolipids w/o post-hydrolysis work-up (~50% active)
⁹ (s) Significant difference is operator noticeable vs Comparative Example w/Commercial Rhamnolipids
* ≥2 cm difference is considered consumer noticeable
** ≥2 point difference is considered consumer noticeable
^{#} demonstrates robust bacteria and fungal reduction

### Test Methods

### 1. Microbial Susceptibility Testing Method

Organisms are prepared for Microbial Susceptibility Testing as follows: Bacteria, including Escherichia coli (ATCC #8739, American Type Culture Collection, Manassas, Virginia, USA), Staphylococcus aureus (ATCC #6538, American Type Culture Collection, Manassas, Virginia, USA), Pseudomonas aeruginosa (ATCC #9027, American Type Culture Collection, Manassas, Virginia, USA), Burkholderia cepacia (ATCC #25416, American Type Culture Collection, Manassas, Virginia, USA), as well as environmental isolates of Klebsiella pneumoniae, Enterobacter gergoviae and Serratia marcescens, are streaked on Tryptic Soy Agar (TSA, Becton Dickinson DIFCO™ Tryptic Soy Agar, Franklin Lakes, NJ, USA) and incubated at 30-35° C. for 18-24 hrs. Candida albicans (ATCC #10231, American Type Culture Collection, Manassas, Virginia, USA) is streaked on Sabouraud Dextrose Agar (SDA, Neogen, Lansing, MI, USA) and incubated at 20-25° C. for 44-52 hrs while Aspergillus brasiliensis (ATCC #16404, American Type Culture Collection, Manassas, Virginia, USA) is streaked on SDA and incubated in a dark 20-25° C. chamber for 6-10 days until dense dark sporulation is observed. Organism suspensions are prepared by transferring confluent growth to saline (0.85% NaCl) or saline with 0.05% Tween 80 (polysorbate 80) (A. brasiliensis only) and turbidometrically adjusted to a target concentration of 10⁷-10⁸ CFU/ml.

Bacterial microbial susceptibility is tested as follows: A bacterial pool (mixture in equal volumes) of challenge organisms, is used in this test and prepared such that the final concentration is approximately 6-8 log cfu/ml. This inoculum is added at a ratio of 1% v/w to product and inoculated products are incubated at 20-25° C. for up to 7 days. Organism survival is measured during and at the end of the incubation period by neutralizing an aliquot of inoculated sample in Modified Letheen Broth containing 1.5% polysorbate 80 and 1% Lecithin (MLBTL). Successive dilutions are transferred into petri dishes containing Modified Letheen Agar with 1.5% Tween 80 (polysorbate 80), and the agar plates are incubated at least 2 days at 30-35° C. Bacterial colony forming units (cfu) are then enumerated, and a bacterial log reduction from the starting log cfu/g challenge level is reported. Greater log cfu/g reduction values indicate greater anti-bacterial robustness.

Fungal microbial susceptibility is tested as follows: Candida albicans and Aspergillus brasiliensis are mixed in equal volumes such that the concentration of the fungal pool is approximately 6-8 log cfu/ml. This inoculum is added at a ratio of 1% v/w to product and inoculated product is incubated at 20-25° C. for up to 14 days. Organism survival is measured during and at the end of the incubation period by neutralizing an aliquot of inoculated sample in Modified Letheen Broth containing 1.5% polysorbate 80 and 1% Lecithin (MLBTL). Successive dilutions are transferred into petri dishes containing Sabouraud Dextrose Agar, and the agar plates are incubated at least 2 days at 30-35° C. Fungal colony forming units (cfu) are then enumerated, and a fungal log reduction from the starting log cfu/g challenge level is reported. Greater log cfu/g reduction values indicate greater anti-fungal robustness.

### 2. Minimal Kill Concentration Assay (MKCA)

The MKC assay is a 96 well plate method to determine the minimum concentration of a test material or composition needed to kill Gram-negative and Gram-positive bacteria (Escherichia coli ATCC 8739 and Staphylococcus aureus ATCC 6538, respectively).

The bacterial suspensions are prepared from 10× freezer stocks with 15% glycerol. The freezer stocks are centrifuged at ˜13,000×g (Eppendorf Centrifuge 5417C No. 541705483) for 1 minute. The supernatant is removed, and the cell pellet is resuspended in 1 mL saline (Sodium chloride solution 0.9%, Cytiva REF: Z1376), and then transferred to 9 mL saline. The target cfu/ml is 10\hat{ }8, which is confirmed via serial diluting and plating on TSA plates (Tryptic Soy Agar-Sigma-Aldrich Cat #22091). Then 50 μL (25 μL if low on test materials) of bacteria is added to each well of a 96 well 0.5 mL V-Bottom plate (Greiner bio-one REF: 786201) to make a bacteria plate, one separate plate for each bacterium.

The dilutions of the test compounds are prepared in a separate 96 well plate. The test compounds are tested at six dilutions in duplicates or with no replication diluted across the entire plate. The test compounds are prepared in Dimethyl Sulfoxide (Sigma Aldrich GR ACS Cat. No: MX1458-6) or sterile water at 2× the intended test concentration, and then serially diluted in the appropriate vehicle 1:2 across the plate using a multichannel. 50 μL (25 μL if low on test materials) from the test compound plate is added to each well in the bacteria plate all at once using an Integra VIAFLOW 96 channel pipette (Model: VIAFLO 96) and the Integra 300 μL Head (Part No. 6103). With addition of the 2× compounds bacteria plate, the compounds are diluted to a 1× test concentration. The addition of the test compounds to the bacteria wells starts the 20-minute contact time. The plates are mixed well using a Mix-Mate (Eppendorf SE).

After 20 minutes, 1 μL from each well of the bacteria plate is transferred to premade Tryptic Soy Agar (TSA) plates (OmniTray Cell Culture Treated w/Lid Sterile, Cat. No: 165218) for spot plating using the Integra VIAFLOW 96 with the Integra 12.5 μL head (Part No. 6101). Then 4 μL from each well of the bacteria/compound plate is transferred to another 96 well plate containing 36 μL of neutralizer broth Modified Letheen Broth (BD Difco Letheen Broth, Modified REF 263010). The contact time is stopped at 20 minutes once the compound plate is neutralized. The plates are mixed well using a Mix-Mate and then 1 μL of the neutralizer+sample mix is transferred to a TSA OmniTray plate using the Integra VIAFLOW and Integra 12.5 μL head. The TSA spot plates are then incubated overnight for 20 hours at 37° C. in an Incubator. This process is done for both bacteria species.

After overnight incubation, the plates are removed from the incubator and the results recorded by observing which dilutions had bacterial kill/growth. The last dilution that had kill for both replicates is recorded as the MKC.

A positive control of Benzalkonium chloride (Sigma Aldrich Cat. No.: 12060-5G) and vehicle controls are always included in the test.

### 3. Minimum Inhibitory Concentration (MIC) Assay

The Minimum Inhibitory Concentration (MIC) Assay is a 96-well high throughput method that determines the minimum concentration of an active required to inhibit the growth of microorganisms.

Organism preparation: Compounds were evaluated against the following microorganisms: Staphylococcus aureus ATCC #6538 (American Type Culture Collection, Manassas, Virginia, USA). To prepare bacteria solutions, bacteria were streaked on Tryptic Soy Agar (TSA) and incubated at 30-35° C. for 18-24 hrs. Confluent growth was transferred to saline (0.85% NaCl) and turbidometrically adjusted to a target concentration of 10⁷-10⁸ CFU/ml. This inoculum solution was further diluted 1:1000 in Tryptic Soy Broth (pH 7) for assay use.
Assay: Two-fold serial dilutions were prepared in Dimethylsulfoxide (DMSO, Thermo Scientific Pierce DMSO, Sequencing Grade) or sterile water for each test sample. In a sterile 96-well plate, each dilution was further diluted in broth inoculum such that each well contained 5% dilution and 95% broth inoculum. Bacteria plates were incubated on an orbital shaker (Heidolph Titramax 1000 Vibrating Platform Shaker, 200-300 rpm) for 24±2 hrs at 35° C. After incubation, the optical density (OD) of each well was measured in a spectrophotometer (Victor Nivo Multimode Plate Reader) at 600 nm. The MIC was determined as the most dilute well with an OD<50% compared to a growth control (≥50% inhibition). To determine the Minimum Biocidal Concentration (MBC), 2 ul from each well was pipetted onto neutralizing agar (Modified Letheen Agar with Tween) and incubated for 24 hrs at 30-35° C. The MBC was determined as the most dilute concentration with no visible growth.

### 4. Lather Assessment Protocol

Lather Blender Demo—The KitchenAid KSB560CU1 food mixer was used to generate shampoo lather in the laboratory. The high shear rates of a blender produce lather from surfactants that is more similar to that obtained on hair under actual shampooing conditions than provided by cylinder shake test methods.

a. Oil Method: 100 mL Water+2 mL Shampoo+1 mL Olive Oil; Blend on “Stir” for 30 sec; Height of lather is recorded; Lather is poured into small bowl, and lather creaminess is rated.

b. Dirt Method: 300 mL Water+3 mL Shampoo+2 g Potting Soil; Blend on “Stir” for 15 sec; Height of lather is recorded.

### 5. Wet Hair ILS (In-Lab Screening) Protocol

This method can be used to determine the cleaning and/or conditioning properties of the personal care compositions herein. In this method, a 20 g hair switch of Caucasian Low Lift Hair Tresses (International Hair Importers and Products, Inc.; Glendale, NY) is wetted with water, treated with a personal care composition and subject to testing in an In-Lab Screening (ILS) sink. The sink has a salon spray head/hose that is held in place but can be directed to run water over a hair tress that hangs from a rod placed over the sink. The tress can be moved in and out of the water as necessary. The water is maintained at a temperature of 38° C. and a flow rate of 5.7 liters per minute. The testing is as follows:
Calibrate ILS sink to 38° C.
Hang the hair tress switch on rod in sink.
Wet hair thoroughly for 30 seconds. Squeegee the hair tress switch once using your index and middle finger (“scissor fingers) from top to bottom to remove excess water. (“Squeegee” means to clamp the tress at the top in between your index and middle finger and stroke down once to remove water.)
Apply 0.1 g/g (product/hair) of the test composition to the front of the switch, from top to bottom.
Milk the hair tress switch for 15 seconds, then flip the bottom of the switch to the top and milk for another 15 seconds. (“Milk” means to grab the top of the tress and stroke it downward while alternating hands to create lather.)
Evaluate Lather Creaminess (look and feel, after 30 seconds of lathering). Scale: 0=No Creaminess-10=Extremely Creamy
Evaluate Lather Combing (with lather still in switch). Using the lowest pressure possible, place comb all the way through the hair (starting at the top) and using a minimal amount of force (comb from top to bottom) comb through hair switch. Scale: 0=hard to comb-10=easy to comb.
Rinse for 30 seconds (while lightly milking the switch).
Squeegee the hair switch tress once with scissor fingers.
Evaluate Slippery Feel. Squeegee once and assess feel. Scale: 0=No Slip-10=Extremely Slippery.
Evaluate Clean Feel Post Rinse. Stroke the hair from top to bottom between the thumb and two fingers with medium pressure. Gauge how clean or dirty the hair feels. Scale: 0=Low (Dirty)-10=High (Clean).

Evaluate Post Rinse Comb. Using the lowest pressure possible, placing comb (wide tooth side) all the way through the hair (front to back), and using a minimal amount of force (top to bottom) comb through hair switch. Scale: 0=Hard-10=Easy.

### 6. Design of Experiment (DOX) Study Regarding Color Formation During the Base Hydrolysis of Rheance One (Table 6 and FIGS. 3 and 4)

### Procedure

1. Label glass vials with codes to identify each reaction.
2. Measure out the 10 g of Rheance® One (Evonik, 50% weight in water) in each vial and record the accurately the actual amount in the excel spreadsheet. (Metler Toledo Balance XS104, calibration ID B01017464).
3. Set the aluminum reaction blocks and temperature probe on the corresponding heating-stirring plates and set the temperature to the target temperature according to table #.
4. Wait for the thermocouple to reach the target temperature.
5. Place a magnetic stirrer in all the vials.
6. Weigh out and add to the vial the corresponding amount of solid sodium hydroxide (Sigma Aldrich, Lot #1812556927) for each reaction.
7. Place vials into the reaction blocks. Document starting time.
8. Once each vial has reached its corresponding desired length of time, remove it from the reaction block. Take a picture of the vial.
9. Immediately after, take a sample, ˜0.5 g, for GC-FID analysis. Place rest of hydrolysate in the vial in the fridge.
10. Continue until all reactions are completed and put away in the refrigerator.

### Color Measurement Using Lovibond Spectrophotometric Colorimeter-Lovibond PFXi 880/L (for Both Color and Peroxide DOX Studies, Tables 6 and 7, FIGS. 3 and 4)

### Procedure

1. Remove hydrolysate vials from the refrigerator. Let them equilibrate to ambient temperature.
2. Select smallest size cuvette, 10 mm path length.
3. Ensure cells are thoroughly clean and dry.
4. Calibration instrument using the appropriate standards.
5. Measure color for each hydrolysate in the vials.

### Hydrogen Peroxide DOX Procedure (Table 7):

1. Label glass vials with codes to identify each reaction.
2. Measure out the 10 g of hydrolysate in each vial and record accurately the actual amount in the excel spreadsheet (Metler Toledo Balance XS104, calibration ID B01017464).
3. Prior to starting experiment, make sure to calculate the amount of 50 wt. % hydrogen peroxide (Thermo Scientific, Lot #A0448055) needed for each reaction.
4. Set the aluminum reaction blocks and temperature probe on the corresponding heating-stirring plates and set the temperature to 35° C.
5. Wait for the thermocouple to reach the target temperature.
6. Place a magnetic stirrer in all the vials.
7. Weigh out and add to the vial the corresponding amount of hydrogen peroxide for each reaction (Metler Toledo Balance XS104, calibration ID B01017464). Document exact amount added.
8. Place vials into the reaction blocks. Document starting time.
9. Once each vial has reached its corresponding desired length of time, remove it from the reaction block. Take a picture of the vial.
10. Immediately after, place vial in the fridge.
11. Continue until all reactions are completed.
   a g

The following Test Method measures the amount of β-hydroxy fatty acid in raw material by GC-FID (Gas Chromatography-flame ionization detection). Fatty acids are derivatized to trimethylsilyl esters.

| INSTRUMENT | SUGGESTED |
|---|---|
| GC with FID | Split/splitless inlet with FID |
| Capillary Column | DB-1 2.5 m × 0.25 mm × 0.25 um |
| Liner | Straight with glass wool |
| APPARATUS | SUGGESTED TYPE |
| Analytical Balance | Accurate to 0.0001 g |
| Microbalance | |
| Positive Displacement Pipette | to deliver 100, 300 uL |
| Heating apparatus capable of 65 C. | Dry Bath Type for 20 ml vials VWR 10753-536 |
| Nitrogen Gas for drying samples | Mini-evaporator Sigma Aldrich 22971 |
| Preparation vials | Glass, 20 mL size recommended |
| REAGENTS AND SOLUTIONS | PURITY AND SOURCE |
| 3-hydroxy dodecanoic acid | Analytical standard, Cayman Chemical |
| 3- hydroxy decanoic acid | Analytical standard, Cayman Chemical |
| tridecanoic acid | Sigma Aldrich 91988-5G |
| pyridine | Sigma Aldrich 270407-100ML |
| BSTFA + 1% TMCS | Sigma Aldrich T6381-100G |
| chloroform | VWR EM-CX1050-1 (EMD) (any HPLC grade or better) |
| Methanol | HPLC grade |

### Procedure

### Preparation of Special Reagents.

1:1 Chlor: MeOH: e.g., 500 mL chloroform plus 500 mL methanol.

### Preparation of Standards

Internal Standard Stock (˜800 μg/ml, not quantitative).
   Weigh 0.050±0.002 g of tridecanoic acid into a scintillation vial accurately recording the weight. Add approximately 10 g of pyridine and accurately record the weight. Mix and dissolve. This expires in 2 weeks. Store at RT.
Calibration Standard Stock
   Weigh 0.01±0.01 g of 3-hydroxy decanoic acid into a scintillation vial accurately recording the weight. Add ˜5 ml of 1:1 Chlor: MeOH and accurately record the weight. Mix and dissolve. This expires in 2 weeks. Store at RT.
Check Standard Stock
   Weigh 0.01 g of 3-hydroxy decanoic acid into a scintillation vial accurately recording the weight. Add ˜5 mL of 1:1 Chlor: MeOH and accurately record the weight. Mix and dissolve.
Proceed with Derivatize Stocks in the Preparation of Samples section.
Measure the density of the 1:1 Chlor: MeOH by accurately weighing 1000 ul from a positive displacement pipette.

### Preparation of Samples

Prepare a Stock for Each Sample:
   Weigh 0.10+0.01 g of sample into a scintillation vial accurately recording the weight.
   Add ˜10 g of 1:1 Chlor: MeOH and accurately record the weight.
   Mix and Dissolve.
Derivatize Stocks:
   Pipet 1000 ul of each stock into an autosampler vial:
      Vial 1: Blank (Chlor: MeOH only)
      Vial 2: ISTD Blank (nothing)
      Vial 3: Working Standard (Calibration Stock)
      Vial 4: Check Standard (Check Stock)
      Vial 5+: Sample Stock
   Dry under nitrogen. When almost dry, transfer to vacuum oven at 60° C. for one hour.
   Pipet 100 μl of the Internal Standard Stock into each vial except Vial 2.
   Pipet 300 ul of BSTFA with 1% TMCS into each vial and cap.
   Double check to make sure caps are tight (septa should be flat and not deformed).
   Heat for 1 hour at 90° C.
   The samples are now ready for GC. Use chloroform as rinse solvent.

### GC-FID Instrument

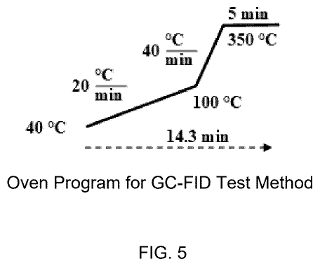

Operate GC in constant flow mode. This maintains the same flow regardless of the column temperature and provides the best efficiency at high temperatures. Follow general procedures for operating the GC using the following parameters, including the oven program as shown in FIG. 5.
Column Flow (He): 1.1 ml/min
Column Flow (H₂): 1.5 ml/min
Sample Volume: 1 ul
(Split Injection) Ratio: 50:1
Injector Temperature: 350° C.
Detector Temperature: 350° C.
Make-up+Column Flow (N₂): 30 ml/min
Detector Hydrogen Flow: 40 mL/min
Detector Air Flow: 400 mL/min

A calibration curve constructed by analyzing β-hydroxy fatty acids at different concentrations and was then used to calculate the B-hydroxy fatty acids in the samples.

### EXAMPLES/COMBINATIONS

A. A composition comprising rhamnolipid compositions, said rhamnolipid compositions comprising (a) and (b), and said composition further comprising (c), wherein (a), (b), and (c) are as follows:

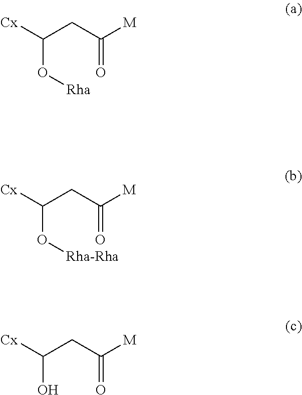

wherein Rha is rhamnose;
wherein each Cx is independently selected from an alkyl, aryl, heteroalkyl, heteroaryl, unsaturated alkenyl, and unsaturated heteroalkenyl;
wherein each Cx independently has a carbon chain length from 4 to 22;
wherein each M is independently selected from OH; O−X+ wherein X+ is a cation; OR1; alkyl, heteroalkyl; aryl; heteroaryl; hetero arylalkyl; arylalkyl; and tauryl;
wherein R1 is selected from an alkyl, branched alkyl, and cyclic alkyl;
   and all possible stereoisomers thereof; and all combinations thereof; and
wherein (a) or (b) is at least about 25 wt % or greater of all rhamnolipids present in the compositions.
   B. The composition of paragraph A, wherein the cation is selected from Na+, K+, Li+, Cs+, +NH₃R2; +NH₂R2R3; +NHR2R3R4, +NR2R3R4R5 wherein R2, R3, R4, and R5 are each independently selected from an alkyl, branched alkyl and cyclic alkyl.
   C. The composition of paragraph A or B, wherein M for (a), (b), and (c) is OH.
   D. The composition of any one of paragraphs A-C, wherein M for (a), (b), and (c) is O−X+.
   E. The composition of any one of paragraphs A-D, wherein each Cx independently has a carbon chain length from 5 to 13.
   F. The composition of any one of paragraphs A-E, wherein each Cx independently has a carbon chain length of 10 or 12.
   G. The composition of any one of paragraphs A-F, wherein (a) or (b) is at least about 80% of the total composition.
   H. The composition of any one of paragraphs A-G, wherein (a) or (b) is at least about 99% of the total composition.
   I. The composition of any one of paragraphs A-H, wherein (a) is at least 80% of the total composition.
   J. The composition of any one of paragraphs A-I, wherein (a) is selected from:
3-(((2R,3R,4R,5R,6S)-3,4,5-trihydroxy-6-methyltetrahydro-2H-pyran-2-yl)oxy)hexadecanoic acid;
3-(((2R,3R,4R,5R,6S)-3,4,5-trihydroxy-6-methyltetrahydro-2H-pyran-2-yl)oxy)octenoic acid;
3-(((2R,3R,4R,5R,6S)-3,4,5-trihydroxy-6-methyltetrahydro-2H-pyran-2-yl)oxy)decenoic acid;
3-(((2R,3R,4R,5R,6S)-3,4,5-trihydroxy-6-methyltetrahydro-2H-pyran-2-yl)oxy)octanoic acid;
3-(((2R,3R,4R,5R,6S)-3,4,5-trihydroxy-6-methyltetrahydro-2H-pyran-2-yl)oxy)tetradecanoic acid;
3-(((2R,3R,4R,5R,6S)-3,4,5-trihydroxy-6-methyltetrahydro-2H-pyran-2-yl)oxy)decanoic acid;
3-(((2R,3R,4R,5R,6S)-3,4,5-trihydroxy-6-methyltetrahydro-2H-pyran-2-yl)oxy)dodecanoic acid;
3-(((2R,3R,4R,5R,6S)-3,4,5-trihydroxy-6-methyltetrahydro-2H-pyran-2-yl)oxy)dodecen-dienoic acid,
methyl 3-(((2R,3R,4R,5R,6S)-3,4,5-trihydroxy-6-methyltetrahydro-2H-pyran-2-yl)oxy)decanoate;
and all possible stereoisomers thereof, and all combinations thereof.
   K. The composition of any one of paragraphs A-J, wherein (b) is selected from:
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)hexadecanoic acid;
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecanoic acid;
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decenoic acid;
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecenoic acid;
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decanoic acid;
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)octanoic acid; and
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)dodecanoic acid.
3-(((2R,3R,4R,5R,6S)-4,5-dihydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)-N-(2-ethylhexyl) decanamide;
N-cyclohexyl-3-(((2R,3R,4R,5R,6S)-4,5-dihydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)decanamide;
3-(((2R,3R,4R,5R,6S)-4,5-dihydroxy-6-methyl-3-(((2S,3R,4R,5R,6S)-3,4,5trihydroxy-6-methyltetrahydro-2H-pyran-2-yl)oxy)tetrahydro-2H-pyran-2-yl)oxy)-N-(2-hydroxyethyl) decanamide;
N,N-dibutyl-3-(((2R,3R,4R,5R,6S)-4,5-dihydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)decanamide;
3-(((2R,3R,4R,5S,6S)-4,5-diacetoxy-6-methyl-3-(((2S,3R,4R,6R)-3,4,5-triacetoxy-6-methyltetrahydro-2H-pyran-2-yl)oxy)tetrahydro-2H-pyran-2-yl)oxy)decanoic acid;
isopropyl 3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)decanoate;
3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)-N,N-dimethyldecanamide;
N-benzyl-3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)decanamide;
3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)-N-methyldecanamide; and all possible stereoisomers thereof, and all combinations thereof.
   L. The composition of any one of paragraphs A-K, wherein (c) is selected from:
   3-hydroxytetradecanoic acid;
   3-hydroxyhexadecanoic acid;
   3-hydroxydeceneoic acid;
   3-hydroxytetradecenoic acid;
   3-hydroxydodecanoic acid;
   3-hydroxydodecenoic acid;
   3-hydroxy octanoic acid;
   3-hydroxy dodec-dienoic acid; and
   3-hydroxy decaneoic acid;
   and all possible stereoisomers thereof, and all combinations thereof.
      M. The composition of any one of paragraphs A-L, wherein (a) is 3-(((2R,3R,4R,5R,6S)-3,4,5-trihydroxy-6-methyltetrahydro-2H-pyran-2-yl)oxy)hexadecanoic acid; (b) is 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)hexadecanoic acid; and (c) is 3-hydroxytetradecanoic acid.
      N. The composition of any one of paragraphs A-M, wherein the composition has an antimicrobial effect.
      O. The composition of any one of paragraphs A-N, wherein the composition has a minimal kill concentration against E. coli of less than about 20,000 ppm and a minimal kill concentration against S. aureus of less than about 35,000 ppm.
      P. The composition of any one of paragraphs A-O, further comprising a di-rhamno-di-lipid.
      Q. The composition of any one of paragraphs A-P, wherein the di-rhamno-di-lipids are at most about 25 wt % of all rhamnolipid compositions present in the composition.
      R. The composition of any one of paragraphs A-Q, comprising: C14, C16, C10:1, C14:1, C12, Rha-RhaC16, Rha-Rha-C14, C12:1, Rha-Rha-C10:1, C8, Rha-C16, RhaC8:1, C12:2, RhaRha-C14:1, RhaRha-C10, Rha-C10:1, RhaRha-C8. RhaRha-C12, RhaRha-C14:1, Rha-C8, Rha-C14; C10, Rha-C10, Rha-C12, and RhaRha-C12:2.
      S. A method of making the composition of any one of paragraphs A-R, comprising the following steps:
   a. providing a rhamnolipid aqueous solution;
   b. reacting the rhamnolipid aqueous solution with a base and/or caustic for 2 to 6 hours at 80° C. to 100° C.;
   c. optionally, removing the water.
      T. A method of making the composition of any one of paragraphs A-R, comprising the following steps:
   a) providing a rhamnolipid aqueous solution;
   b) reacting the rhamnolipid aqueous solution with a base and/or caustic for 0.25 to 100 hours at 25° C. to 100° C.;
   c) optionally, removing the water.
      U. A method of making the composition of any one of paragraphs A-T, further comprising a step of treating the composition with hydrogen peroxide.
      V. A composition consisting of (a), (b), and (c), wherein (a) and (b) are rhamnolipid compositions and (a), (b), and (c) are as follows:

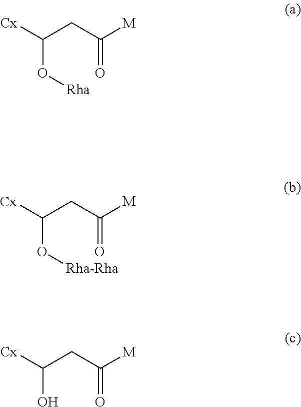

wherein Rha is rhamnose;
wherein each Cx is independently selected from an alkyl, aryl, heteroalkyl, heteroaryl, unsaturated alkenyl, and unsaturated heteroalkenyl;
wherein each Cx independently has a carbon chain length from 4 to 22;
wherein each M is independently selected from OH; O−X+ wherein X+ is a cation; OR1; alkyl, heteroalkyl; aryl; heteroaryl; hetero arylalkyl; arylalkyl; and tauryl;
wherein R1 is selected from an alkyl, branched alkyl, and cyclic alkyl;
and all possible stereoisomers thereof; and
wherein (a) or (b) is at least about 25 wt % of all rhamnolipid compositions.
   W. A composition consisting essentially of: (a), (b), and (c), wherein (a) and (b) are rhamnolipid compositions and (a), (b), and (c) are as follows:

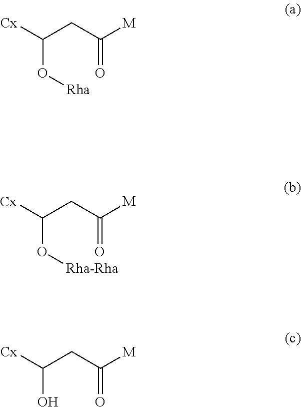

wherein Rha is rhamnose;
wherein each Cx is independently selected from an alkyl, aryl, heteroalkyl, heteroaryl, unsaturated alkenyl, and unsaturated heteroalkenyl;
wherein each Cx independently has a carbon chain length from 4 to 22;
wherein each M is independently selected from OH; O−X+ wherein X+ is a cation; OR1; alkyl, heteroalkyl; aryl; heteroaryl; hetero arylalkyl; arylalkyl; and tauryl;
wherein R1 is selected from an alkyl, branched alkyl, and cyclic alkyl;
and all possible stereoisomers thereof; and
wherein (a) or (b) is at least about 25 wt % of all rhamnolipid compositions.
   X. A composition comprising rhamnolipid compositions, said rhamnolipid compositions comprising (a) mono-rhamno-mono-lipids and (b) di-rhamno-mono-lipids; said composition further comprising (c) beta-hydroxy-fatty acid;
   wherein (a) or (b) comprise at least about 25 wt % of the total rhamnolipid compositions.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising rhamnolipid compositions, said rhamnolipid compositions comprising (a) and (b), and said composition further comprising (c), wherein (a), (b), and (c) are as follows: 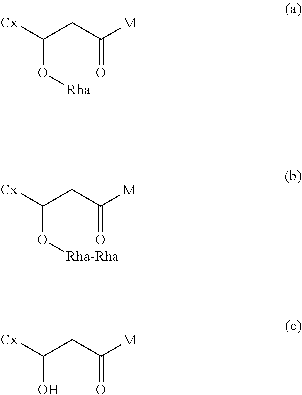
wherein Rha is rhamnose;
wherein each Cx is independently selected from an alkyl, aryl, heteroalkyl, heteroaryl, unsaturated alkenyl, and unsaturated heteroalkenyl;
wherein each Cx independently has a carbon chain length from 4 to 22;
wherein each M is independently selected from OH; O−X+ wherein X+ is a cation; OR1; alkyl; heteroalkyl; aryl; heteroaryl; hetero arylalkyl; arylalkyl; and tauryl;
wherein R1 is selected from an alkyl, branched alkyl, and cyclic alkyl; and combinations thereof; and all possible stereoisomers thereof; and
wherein (a) or (b) is at least about 25 wt % or greater of all rhamnolipids present in the compositions.

2. The composition of claim 1, wherein the cation is selected from Na+, K+, Li+, Cs+, +NH₃R2; +NH₂R2R3; +NHR2R3R4, +NR2R3R4R5 wherein R2, R3, R4, and R5 are each independently selected from an alkyl, branched alkyl, and cyclic alkyl.

3. The composition of claim 1, wherein M for (a), (b), and (c) is OH.

4. The composition of claim 1, wherein M for (a), (b), and (c) is O−X+.

5. The composition of claim 1, wherein each Cx independently has a carbon chain length from 5 to 13.

6. The composition of claim 1, wherein each Cx independently has a carbon chain length of 10 or 12.

7. The composition of claim 1, wherein (a) or (b) is at least about 80% of the total composition.

8. The composition of claim 1, wherein (a) or (b) is at least about 99% of the total composition.

9. The composition of claim 1, wherein (a) is at least 80% of the total composition.

10. The composition of claim 1, wherein (a) is selected from:
3-(((2R,3R,4R,5R,6S)-3,4,5-trihydroxy-6-methyltetrahydro-2H-pyran-2-yl)oxy)hexadecanoic acid;
3-(((2R,3R,4R,5R,6S)-3,4,5-trihydroxy-6-methyltetrahydro-2H-pyran-2-yl)oxy)octenoic acid;
3-(((2R,3R,4R,5R,6S)-3,4,5-trihydroxy-6-methyltetrahydro-2H-pyran-2-yl)oxy)decenoic acid;
3-(((2R,3R,4R,5R,6S)-3,4,5-trihydroxy-6-methyltetrahydro-2H-pyran-2-yl)oxy)octanoic acid;
3-(((2R,3R,4R,5R,6S)-3,4,5-trihydroxy-6-methyltetrahydro-2H-pyran-2-yl)oxy)tetradecanoic acid;
3-(((2R,3R,4R,5R,6S)-3,4,5-trihydroxy-6-methyltetrahydro-2H-pyran-2-yl)oxy)decanoic acid;
3-(((2R,3R,4R,5R,6S)-3,4,5-trihydroxy-6-methyltetrahydro-2H-pyran-2-yl)oxy)dodecanoic acid; and
3-(((2R,3R,4R,5R,6S)-3,4,5-trihydroxy-6-methyltetrahydro-2H-pyran-2-yl)oxy)dodecen-dienoic acid,
methyl 3-(((2R,3R,4R,5R,6S)-3,4,5-trihydroxy-6-methyltetrahydro-2H-pyran-2-yl)oxy)decanoate;
and all possible stereoisomers thereof, and all combinations thereof.

11. The composition of claim 1, wherein (b) is selected from:
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)hexadecanoic acid;
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecanoic acid;
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decenoic acid;
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecenoic acid;
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decanoic acid;
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)octanoic acid; and
3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)dodecanoic acid,
3-(((2R,3R,4R,5R,6S)-4,5-dihydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)-N-(2-ethylhexyl) decanamide;
N-cyclohexyl-3-(((2R,3R,4R,5R,6S)-4,5-dihydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)decanamide;
3-(((2R,3R,4R,5R,6S)-4,5-dihydroxy-6-methyl-3-(((2S,3R,4R,5R,6S)-3,4,5trihydroxy-6-methyltetrahydro-2H-pyran-2-yl)oxy)tetrahydro-2H-pyran-2-yl)oxy)-N-(2-hydroxyethyl) decanamide;
N,N-dibutyl-3-(((2R,3R,4R,5R,6S)-4,5-dihydroxy-6-methyl-3-(((2S,3R,4R,5R,6S)-3,4,5-trihydroxy-6-methyltetrahydro-2H-pyran-2-yl)oxy)tetrahydro-2H-pyran-2-yl)oxy)decanamide;
3-(((2R,3R,4R,5S,6S)-4,5-diacetoxy-6-methyl-3-(((2S,3R,4R,6R)-3,4,5-triacetoxy-6-methyltetrahydro-2H-pyran-2-yl)oxy)tetrahydro-2H-pyran-2-yl)oxy)decanoic acid;
isopropyl 3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)decanoate;
3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)-N,N-dimethyldecanamide;
N-benzyl-3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)decanamide;
3-(((2R,3R,4R,5R,6S)-4,5-dihydroxy-6-methyl-3-(((2S,3R,4R,6R)-3,4,5-trihydroxy-6-methyltetrahydro-2H-pyran-2-yl)oxy)tetrahydro-2H-pyran-2-yl)oxy)-N-methyldecanamide;
and all possible stereoisomers thereof, and all combinations thereof.

12. The composition of claim 1, wherein (c) is selected from:
3-hydroxytetradecanoic acid;
3-hydroxyhexadecanoic acid;
3-hydroxydeceneoic acid;
3-hydroxytetradecenoic acid;
3-hydroxydodecanoic acid;
3-hydroxydodecenoic acid;
3-hydroxyoctanoic acid;
3-hydroxydodec-dienoic acid; and
3-hydroxydecaneoic acid, and all possible stereoisomers thereof, and all combinations thereof.

13. The composition of claim 1, wherein (a) is 3-(((2R,3R,4R,5R,6S)-3,4,5-trihydroxy-6-methyltetrahydro-2H-pyran-2-yl)oxy)hexadecanoic acid; (b) is 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)hexadecanoic acid; and (c) is 3-hydroxytetradecanoic acid.

14. The composition of claim 1, wherein the composition has an antimicrobial effect.

15. The composition of claim 14, wherein the composition has a minimal kill concentration against E. coli of less than about 20,000 ppm and a minimal kill concentration against S. aureus of less than about 35,000 ppm.

16. The composition of claim 1, further comprising a di-rhamno-di-lipid.

17. The composition of claim 16, wherein the di-rhamno-di-lipids are at most about 25 wt % of all rhamnolipid compositions present in the composition.

18. The composition of claim 1, comprising: C14, C16, C10:1, C14:1, C12, Rha-RhaC16, Rha-Rha-C14, C12:1, Rha-Rha-C10:1, C8, Rha-C16, RhaC8:1, C12:2, RhaRha-C14:1, RhaRha-C10, Rha-C10:1, RhaRha-C8. RhaRha-C12, RhaRha-C14:1, Rha-C8, Rha-C14; C10, Rha-C10, Rha-C12, and RhaRha-C12:2.

19. A method of making the composition of claim 1, comprising the following steps:
a) providing a rhamnolipid aqueous solution;
b) reacting the rhamnolipid aqueous solution with a base and/or caustic for 2 to 6 hours at 80° C. to 100° C.;
c) optionally, removing the water.

20. A method of making the composition of claim 1, comprising the following steps:
a) providing a rhamnolipid aqueous solution;
b) reacting the rhamnolipid aqueous solution with a base and/or caustic for 0.25 to 100 hours at 25° C. to 100° C.;
c) optionally, removing the water.

21. The method of claim 20, further comprising a step of treating the composition with hydrogen peroxide.

22. A composition consisting of (a), (b), and (c), wherein (a) and (b) are rhamnolipid compositions and (a), (b), and (c) are as follows: 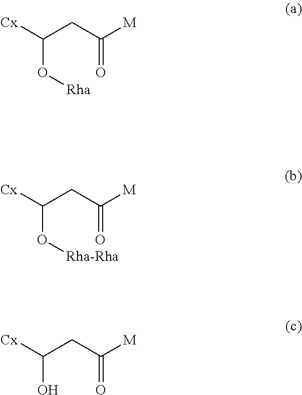
wherein Rha is rhamnose;
wherein each Cx is independently selected from an alkyl, aryl, heteroalkyl, heteroaryl, unsaturated alkenyl, and unsaturated heteroalkenyl;
wherein each Cx independently has a carbon chain length from 4 to 22;
wherein each M is independently selected from OH; O−X+ wherein X+ is a cation; OR1; alkyl, heteroalkyl; aryl; heteroaryl; hetero arylalkyl; arylalkyl; and tauryl;
wherein R1 is selected from an alkyl, branched alkyl, and cyclic alkyl;
and all possible stereoisomers thereof; and
wherein (a) or (b) is at least about 25 wt % of all rhamnolipid compositions.

23. A composition consisting essentially of: (a), (b), and (c), wherein (a) and (b) are rhamnolipid compositions and (a), (b), and (c) are as follows: 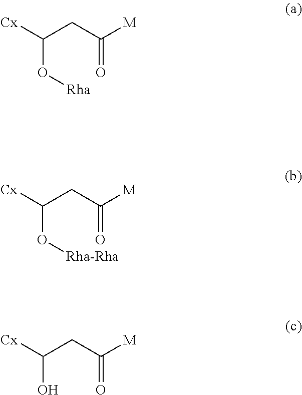
wherein Rha is rhamnose;
wherein each Cx is independently selected from an alkyl, aryl, heteroalkyl, heteroaryl, unsaturated alkenyl, and unsaturated heteroalkenyl;
wherein each Cx independently has a carbon chain length from 4 to 22;
wherein each M is independently selected from OH; O−X+ wherein X+ is a cation; OR1; alkyl, heteroalkyl; aryl; heteroaryl; hetero arylalkyl; arylalkyl; and tauryl;
wherein R1 is selected from an alkyl, branched alkyl, and cyclic alkyl;
and all possible stereoisomers thereof; and
wherein (a) or (b) is at least about 25 wt % of all rhamnolipid compositions.

24. A composition comprising rhamnolipid compositions, said rhamnolipid compositions comprising (a) mono-rhamno-mono-lipids and (b) di-rhamno-mono-lipids; said composition further comprising (c) beta-hydroxy-fatty acids;
wherein (a) or (b) comprise at least about 25 wt % of the total rhamnolipid compositions.
